(19) United States
(12) Patent Application Publication
(10) Pub. No.: US 2022/0118004 A1
(43) Pub. Date: Apr. 21, 2022

(54) TUNABLE IMMUNE RESPONSIVE BIODEGRADABLE ARGININE-BASED POLY(ESTER AMIDE)S AND METHODS OF MAKING AND USING SAME

(71) Applicant: CORNELL UNIVERSITY, Ithaca, NY (US)

(72) Inventors: Chih-Chang Chu, Ithaca, NY (US); Mingyu He, Pittsburgh, PA (US)

(21) Appl. No.: 17/512,124

(22) Filed: Oct. 27, 2021

Related U.S. Application Data
(62) Division of application No. 17/255,242, filed on Dec. 22, 2020.
(60) Provisional application No. 62/690,773, filed on Jun. 27, 2018.

(51) Int. Cl. A61K 31/785; A61P 17/02; A61L 15/26

(52) U.S. Cl. CPC A61K 31/785; A61L 15/26; A61P 17/02

(57) ABSTRACT

The present disclosure provides poly(ester amide)s (PEAs) containing NO-Arg groups. The PEAs may be used to treat diseases having inflammation and/or diseases associated with inflammation. The PEAs may be used to treat wounds, such as, for example, wounds associated with obesity and/or diabetes. The PEAs may be incorporated into various articles such as medical devices, such as, for example, bandages and other wound care articles/devices.

### CROSS REFERENCE TO RELATED APPLICATIONS

This application claims priority to U.S. Provisional Application No. 62/690,773, filed on Jun. 27, 2018, the disclosure of which is herein incorporated by reference.

### BACKGROUND OF THE DISCLOSURE

Inflammation is associated with all diseases. The ability to modulate/control inflammation may provide a new therapeutic approach towards the treatment of inflammation-associated diseases.

Inflammation is often associated with wound repair. The process of wound repair is a series of ordered temporally regulated biochemical and cellular events. Many of these events that occur within the wound provide metabolic substrates for optimal cell function and regulation of inflammation, granulation tissue formation (proliferative), and remodeling stages. During the wound healing process, two distinct enzymes, nitric oxide synthase (NOS) and arginase, are utilized during L-arginine (Arg) metabolism by macrophages at different times. Arg may be metabolized by macrophages via either the NOS route to produce nitric oxide (NO) and L-citrulline, or by arginase to produce L-ornithine, polyamines, and urea. The effect of NO on wound repair are diverse involving, for example, angiogenesis, inflammation, cell proliferation, matrix deposition, remodeling and mediating apoptosis. Ornithine, produced through the arginase action, is a precursor for proline which serves as the substrate for collagen synthesis, whereas polyamines are involved in cell proliferation.

The common substrate for both pathways, Arg, is a conditionally essential amino acid that must be supplied in the diet during certain physiological or pathological conditions, such as pregnancy, sepsis, and trauma, in which the requirement exceeds the production capability. The concentration of Arg in the wound milieu may decrease to undetectable levels that limit the production of NO, L-ornithine, polyamines, and the like, from both NOS and arginase pathways for either inflammatory and/or wound healing process. Arg supplement increases NO synthesis, collagen synthesis and stimulates fibroblast proliferation. NO level administration using a NO donor drug (S-nitrosoglutathione) in both inflammatory and proliferative stages improves cutaneous wound repair. However, a continuous infusion of Arg into a local wound environment produced NO, with an associated reduction in vascular endothelial growth factor (VEGF) content and less angiogenesis. An uncontrolled production of NO generated from Arg supplement through iNOS pathway can be detrimental and lead to excessive inflammation, impaired cellular respiration, non-specific cytotoxicity, etc. A high NO level also reduces the number of myofibroblasts by inhibiting replication or by influencing the cell differentiation and reduce the expression of intracellular collagen I of fibroblasts and myofibroblasts.

Macrophages originate mainly from circulating monocytes, and play an important role in wound healing. Macrophages contribute to NO production, fibrin dissolution, the removal of dead tissue and ingrowth of new blood vessels, fibroblast recruitment, connective-tissue remodeling, synthesizing and release a vast array of regulatory molecules relevant to wound healing, including transforming growth factor-β1 (TGF-β1) and VEGF which are essential to wound repair. The iNOS activity of the classically activated macrophages (CAM) dominates and produces elevated levels of NO. Alternatively, activated macrophages (AAM) by interleukin-4 (IL-4) or interleukin-13 (IL-13) promote the arginase-dependent formation of L-ornithine. The late suppression of iNOS pathway and the subsequent catabolism of Arg through arginase are associated with the transition of the biofunction of macrophages from pro-inflammatory to pro-healing. The phagocytosis of apoptotic neutrophils by macrophages induces the release of TGF-β which inhibits iNOS activity, increase arginase activity and reduces the production of pro-inflammatory mediators. During the resolution of the inflammation stage of healing, arginase is released from apoptotic macrophages and produced ornithine-derived collagen precursors (glutamic semialdehyde->proline/hydroproline) which might be utilized by fibroblast and myofibroblast to synthesize collagen in the subsequent granulation tissue formation stage in wound healing. After the early inflammatory stage, TGF-β1, cellular fibronectin and mechanical stress lead to the transition from fibroblasts to myofibroblasts within granulation tissue. In an open wound, fibroblasts and myofibroblasts produce provisional extracellular matrix (ECM) and myofibroblast provide contraction and bring the wound margins closer. In normal wound repair, the macrophage and myofibroblast cell numbers are dramatically reduced via apoptosis at the resolution phase of inflammation and granulation tissue formation, respectively.

Chronic wound healing greatly differs from and normal wound healing in conditions surrounding the injury. Persistent inflammatory response (including elevated and persistent NO level), impaired inflammatory cell function and altered cytokines and growth factor concentrations are usually associated with chronic wounds. One common case of impaired chronic wound healing is diabetic wounds. Suppressing the overproduction of NO, reactive oxygen species (ROS)/reactive nitrogen species (RNS) derived from NO, rebalancing the pro-inflammatory cytokines level and protease activity may facilitate the resolution of inflammation stage, hence improve the chronic wound healing.

N-nitro-L-arginine (NOArg) is a competitive inhibitor of all NOS with low toxicity and excellent stability in an aqueous environment. The major limiting factor of NOArg application in biological systems is its low water solubility (˜0.87 mg/mL) at a neutral pH. N-nitro-L-arginine methyl ester (L-NAME) is another more commonly used NOS inhibitor with better water solubility but less potent. Some studies reported proper L-NAME administration lower NO concentration at the injury site so as to enhance hydroxyproline/collagen synthesis and to lower inflammatory response. Due to the important and many-faceted function of NO in wound healing, high dose of L-NAME administration could also reduce inflammatory cell recruitment and migration, hence led to impaired wound contraction. Using a proper combination of NOS inhibitor and Arg as a topical treatment on the chronic wound site may achieve a better healing by altering the balance of NOS/arginase metabolism pathways at the injury site. However, the treatment by a simple application of a solution formulation of water-soluble small-molecule NOS inhibitor and Arg on an open wound during the inflammatory/granulation tissue formation stages would be expected to have a limited efficacy because such a treatment can only achieve a desired NO level in the wound for a short period of time, whereas wound healing is a slow process from several days to weeks. NOS inhibitor supplemented via oral or intravenous administration could also cause unwanted systemic side-effects from multiple cell types and organ systems, because of its nonspecific NOS inhibitory effect.

Thus, there exists an ongoing and unmet need for improved treatments for wounds, inflammation, and diseases associated with inflammation.

### SUMMARY OF THE DISCLOSURE

The present disclosure provides polymers, which includes copolymers and homopolymers (e.g., poly(ester amides) (PEAs)), methods of making same, and uses of same. The copolymers may be referred to as polymers. The PEAs may be referred to as amino acid-based poly(esters) (AA-PEAs) or tunable immune-responsive (TIR) AA-PEAs. The polymers of the present disclosure may inhibit nitric oxide synthase in an individual.

In an aspect, the present disclosure provides polymers. Polymers have at least one nitro guanidinium group. Polymers of the present disclosure may be made by methods described in the Examples provided herein.

A poly(ester amide) (PEA) polymer may comprise a first pendant group, where the first pendant group comprises an alkyl nitroguanidine, and at least a second pendant group chosen from an alkyl guanidine group and a side chain of a canonical amino acid, wherein the alkyl chain of the alkyl nitroguanidine group and alkyl guanidine group is 1 to 6 carbon atoms. In various examples, 10 to 90% of the pendant groups is alkyl nitroguanidine groups, including every 0.1% value and range therein. In various examples, the second pendant group is the side chain of arginine. In various other examples, the second or a third pendant group is the side chain of a hydrophobic amino acid or a cross-linking group.

In an example, the polymers of the present disclosure have the following structure:

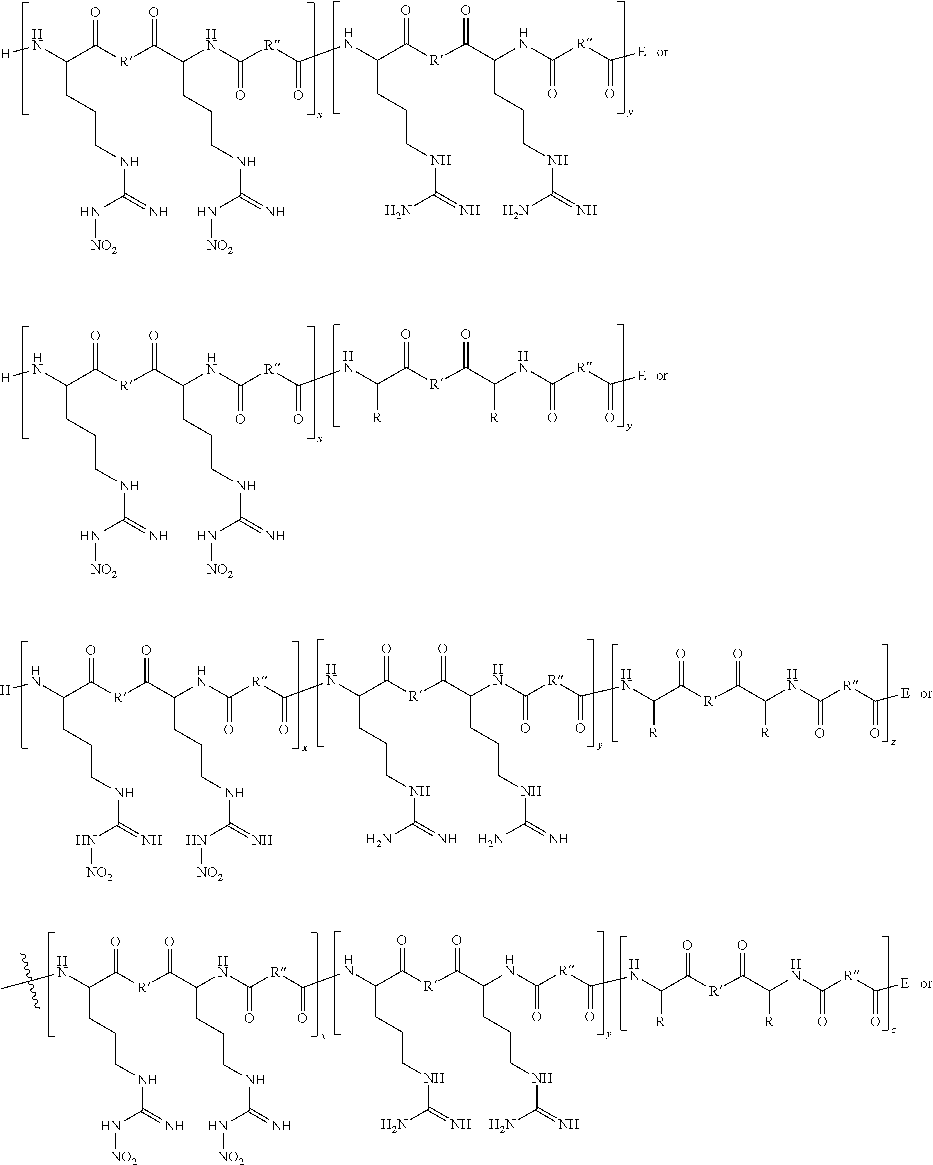

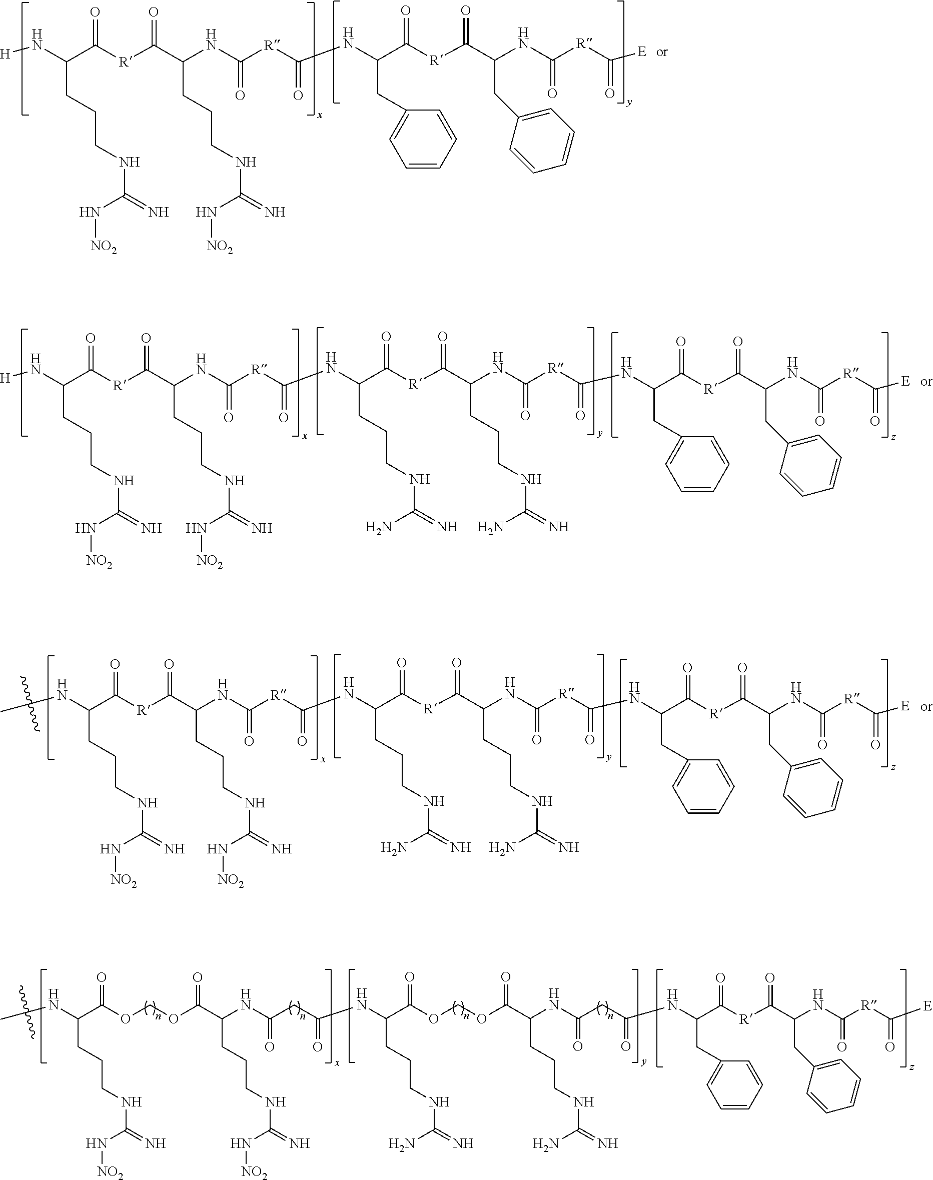

or a salt thereof,
where R at each occurrence is independently chosen from the side chain of a hydrophobic amino acid (e.g., Leu, Gly, Ala, Val, Ile, Pro, Met, Trp) and a cross-linking group (e.g., a vinyl group, alkyl vinyl group, and the like), R′ at each occurrence in the compound is independently chosen from

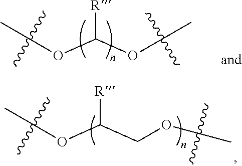

where R′″ at each occurrence in the compound is independently chosen from H and an alkyl group, and where n is 1-10 (1, 2, 3, 4, 5, 6, 7, 8, 9, 10), including all integer values and ranges therebetween (e.g., 1-4 (1, 2, 3, 4)). R″ at each occurrence in the polymer is independently chosen from

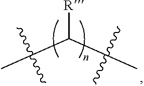

where R′″ at each occurrence in the polymer is independently chosen from H and an alkyl group, and n is 1-10 (1, 2, 3, 4, 5, 6, 7, 8, 9, 10), including all integer values and ranges therebetween (e.g., 1-4 (1, 2, 3, 4)). E is chosen from —H,

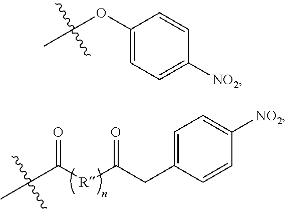

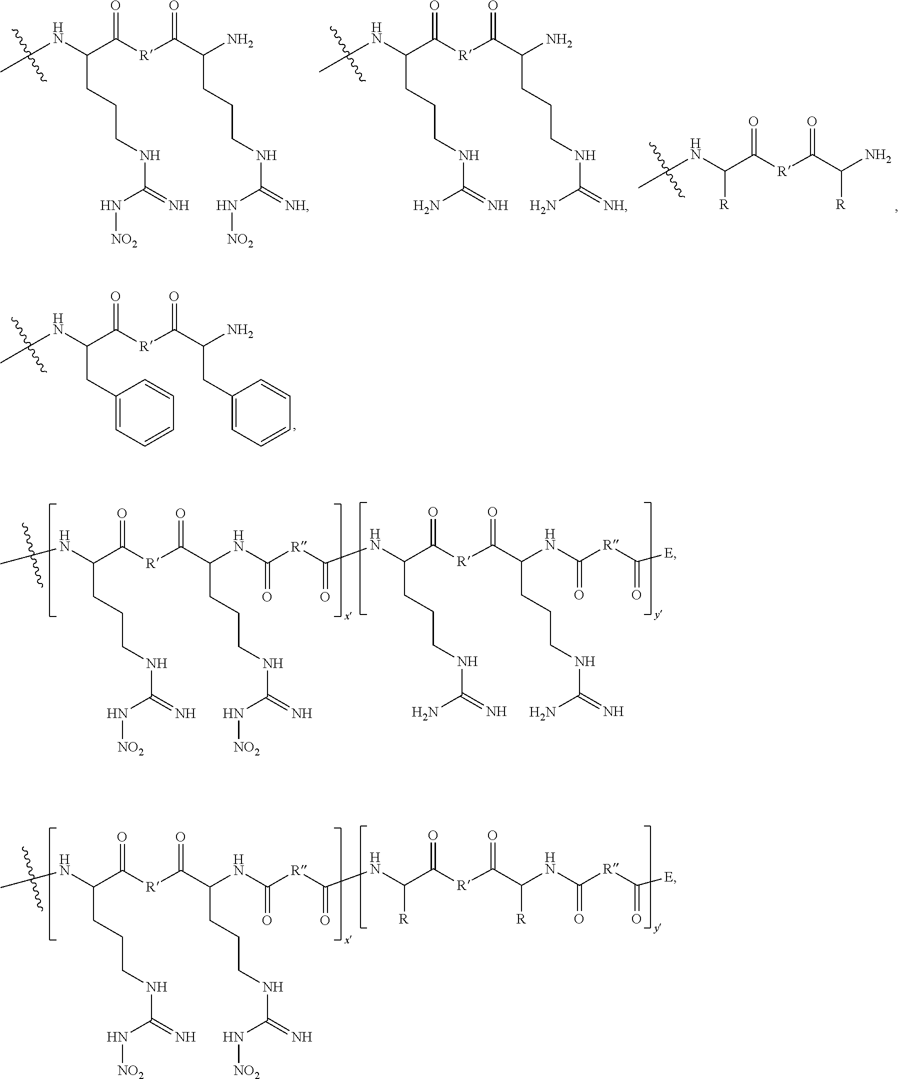

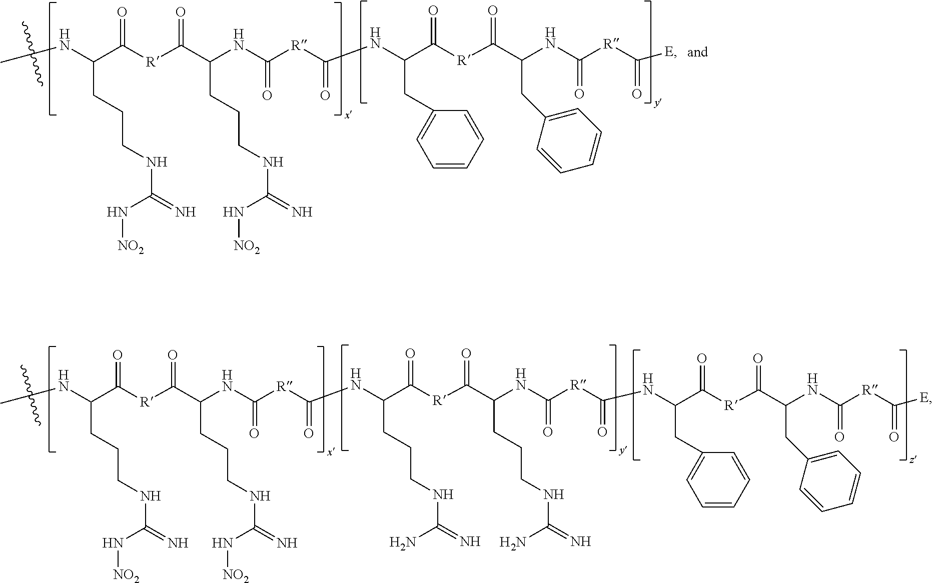

where R at each occurrence is independently chosen from the side chain of a hydrophobic amino acid and a cross-linking group (e.g., a vinyl group, alkyl vinyl group, and the like); R′ at each occurrence in the polymer is independently chosen from

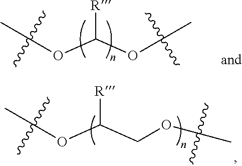

where R′″ at each occurrence in the polymer is independently chosen from H and an alkyl group, and where n is 1-10 (1, 2, 3, 4, 5, 6, 7, 8, 9, 10), including all integer values and ranges therebetween (e.g., 1-4 (1, 2, 3, 4)). R″ at each occurrence in the polymer is independently chosen from

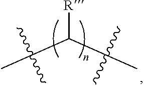

where R′″ at each occurrence in the polymer is independently chosen from H and an alkyl group, and n is 1-10 (1, 2, 3, 4, 5, 6, 7, 8, 9, 10), including all integer values and ranges therebetween (e.g., 1-4 (1, 2, 3, 4). x, y, z′, x′, y′, and z′ are independently at each occurrence 0-100, including all integer values and ranges therebetween. In such a polymer,

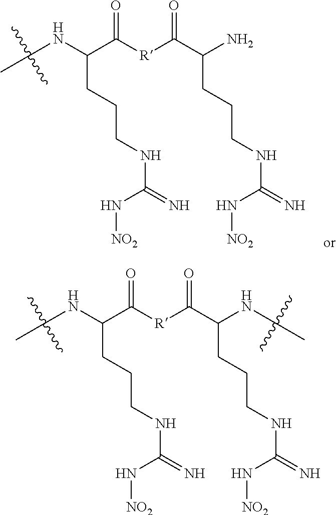

occurs at least once. The stereochemistry may be L or D.

In an aspect, the present disclosure provides compositions. Compositions of the present disclosure comprise at least one polymer of the present disclosure and an acceptable pharmaceutical carrier.

In an aspect, the present disclosure provides methods of using the polymers and/or compositions of the present disclosure. The polymers and/or compositions can be used, for example, in methods of treatment.

In various examples, the present disclosure provides a method of treating a condition (e.g., inflammation and conditions and diseases associated with inflammation or inflammation is a cause), including administering to a subject in need of such treatment a therapeutically effective amount of a composition or polymer of the present disclosure. The polymers and/or compositions may be used to treat diseases associated with, caused by, and/or causing inflammation. Examples of diseases and conditions include, but are not limited to, diabetes, allergies, asthma, atherosclerosis, Crohn's disease, arthritis, obesity, and the like, and combinations thereof. A subject in need of treatment may be in need of treatment for various injuries, various disorders, various wounds, and various diseases.

In an aspect, the disclosure provides kits. A kit may comprise pharmaceutical preparations containing any one or any combination of polymers of the present disclosure and printed material.

In an aspect, the present disclosure provides articles of manufacture. The articles of manufacture may comprise polymers and compositions of the present disclosure. For example, an article of manufacture is a medical device, such as, for example, a bandage, a wound dressing, wound care tool, films, and the like.

### BRIEF DESCRIPTION OF THE FIGURES

For a fuller understanding of the nature and objects of the disclosure, reference should be made to the following detailed description taken in conjunction with the accompanying figures.

FIG. 1 shows chemical reactions of (A) synthesis of bis (NOArg) butane diester monomer (NOArg-4); (B) Synthesis of NOArg-Arg PEA copolymers.

FIG. 2 shows chemical structure characterizations of NOArg-4 monomer, 2-NOArg-4 PEA, and NOArg-Arg PEAs. (A) FTIR of (a) L-Arg hydrochloride, (b) NOArg, (c) L-NAME, (d) NOArg-4 (synthesized in benzene), (e) NOArg-4 (synthesized in toluene); (B) H-NMR spectra of NOArg-4 (synthesized in benzene); (C) FTIR spectra of (f) 2-Arg-4 PEA; (g) 2-NOArg-4-Arg-4 20/80 PEA; (h) 2-NOArg-4-Arg-4 50/50 PEA; (i) 2-NOArg-4 PEA.

FIG. 3 shows an in vitro degradation profile of NOArg PEA, 20/80 PEA and 50/50 PEA in 0.1 M pH 7.4 PBS at 20° C. or 37° C.

FIG. 4 shows 24 hours (h) and 48 h cell viability of RAW 264.7 macrophages (A) and 3T3 fibroblasts (B) incubated with NOArg-4 monomer, 2-NOArg-4, 2-NOArg-4-Arg-4 50/50, 2-NOArg-4-Arg-4 20/80 or 2-Arg-4 PEA at 2 mg/mL or 1 mg/mL concentration. p<0.05.

FIG. 5 shows (A) NO production and (B) Arginase activity of 4×10⁵ macrophages (resting, CAM, alternatively activated) treated with 1 mg/mL or 2 mg/mL Arg, L-NAME, Arg+L-NAME (1/1), NOArg-4 monomer, 2-NOArg-4 PEA, 2-NOArg-4-Arg-4 50/50 PEA or 2-NOArg-4-Arg-4 20/80 PEA or 2-Arg-4 PEA in DMEM media contains 5% FBS in 24 h. **p<0.01, *p<0.05, vs. CAM (LPS+IFN-γ). ##p<0.01, #p<0.05, vs. AAM (IL-4). From left to right the columns in each series are resting macrophages+1 mg/mL treatment, resting macrophages+2 mg/mL treatment, CAM+1 mg/mL treatment, CAM+2 mg/mL treatment, AAM+1 mg/mL treatment, and AAM+2 mg/mL treatment.

FIG. 6 shows (A) TNF-α production and (B) TGF-β1 production of 4×10⁵ macrophages (resting, M1, M2) treated with 1 mg/mL or 2 mg/mL Arg, L-NAME, Arg+L-NAME (1/1), NOArg-4 monomer, 2-NOArg-4 PEA, 2-NOArg-4-Arg-4 50/50 PEA or 2-NOArg-4-Arg-4 20/80 PEA or 2-Arg-4 PEA in DMEM media contains 5% FBS in 24 h. **p<0.01, *p<0.05, vs. CAM (LPS+IFN-γ); #p<0.05, vs. AAM (IL-4). From left to right the columns in each series are resting macrophages+1 mg/mL treatment, resting macrophages+2 mg/mL treatment, CAM+1 mg/mL treatment, CAM+2 mg/mL treatment, AAM+1 mg/mL treatment, and AAM+2 mg/mL treatment.

FIG. 7 shows (A) Representative images of the full-thickness wound beds treated with 2-NOArg-4 PEA, 2-NOArg-4-Arg-4 50/50 PEA, 2-NOArg-4-Arg-4 20/80 PEA, and 2-Arg-4 PEA; (B) the wound closure ratios of various treatments of day 3, 7, and 14 (n=4). * p<0.05 vs control. From left to right the columns in each series are control, Arg PEG, NOArg PEA, 50/50 PEA, and 20/80 PEA.

FIG. 8 shows representative histological H&E stained sections of wounds of diabetic rats treated by NOArg-PEA homopolymer and NOArg-Arg coPEA copolymer biomaterials. (A) at days 3, 7 and 14; (B) H&E staining of wound sections of day 14.

FIG. 9 shows immunohistochemical staining of iNOS (A) and arginase I (B) expression of diabetic wound bed on day 3 and day 7 after a variety of Arg-based PEA treatments.

FIG. 10 shows representative histological Masson's trichrome stained sections of wound bed of diabetic rats treated by a variety of Arg-based biomaterials after days 3, 7, and 14. The dark blue on the left of each image is the original collagen from the unwounded tissue adjacent to the wound site.

FIG. 11 shows in vitro degradation profile of NOArg-PEA, 20/80-PEA and 50/50-PEA in 0.1 M pH 7.4 PBS at 20° C. or 37° C.

FIG. 12 shows NO production of 4×10⁵ macrophages (resting, CAM, AAM) treated with 1 mg/mL or 2 mg/mL Arg, L-NAME, Arg+L-NAME (1/1), NOArg-4 monomer, 2-NOArg-4 PEA, 2-NOArg-4-Arg-4 50/50-PEA, 2-NOArg-4-Arg-4 20/80-PEA, or 2-Arg-4 PEA in DMEM media contains 5% FBS in 24 h. **p<0.01, *p<0.05, vs. CAM (LPS+IFN-γ). ##p<0.01, #p<0.05, vs. AAM (IL-4). From left to right the columns in each series are resting macrophages+1 mg/mL treatment, resting macrophages+2 mg/mL treatment, CAM+1 mg/mL treatment, CAM+2 mg/mL treatment, AAM+1 mg/mL treatment, and AAM+2 mg/mL treatment.

FIG. 13 shows TNF-α production of 4×10⁵ macrophages (resting, CAM, AAM) treated with 1 mg/mL or 2 mg/mL Arg, L-NAME, Arg+L-NAME (1/1), NOArg-4 monomer, 2-NOArg-4 PEA, 2-NOArg-4-Arg-4 50/50-PEA, 2-NOArg-4-Arg-4 20/80-PEA, or 2-Arg-4 PEA in DMEM media contains 5% FBS in 24 h. **p<0.01, *p<0.05, vs. CAM (LPS+IFN-γ); #p<0.05, vs. AAM (IL-4). From left to right the columns in each series are resting macrophages+1 mg/mL treatment, resting macrophages+2 mg/mL treatment, CAM +1 mg/mL treatment, CAM+2 mg/mL treatment, AAM+1 mg/mL treatment, and AAM+2 mg/mL treatment.

FIG. 14 shows the wound closure ratios of various treatments of day 3, 7 and 14 (n=4). * p<0.05, ** p<0.01. From left to right the columns in each series are control, Arg-PEA, NOArg-PEA, 50/50-PEA, 20/80-PEA.

FIG. 15 shows normalized immune response scores of the representative healing wound sections on day 3 (A) and day 7 (B) (n=3). From left to right the columns in each series are iNOS, CD80, Arginase I, CD206.

FIG. 16 shows the collagen content change of the wounded bed of diabetic rats treated by a variety of PEA biomaterials after days 3, 7 and 14. The ratio of the wound neo-tissue collagen content to normal tissue collagen content of the same rat (n=3). *p<0.05. From left to right the columns in each series are control, NOArg-PEA, 50/50-PEA, 20/80-PEA, and Arg-PEA.

FIG. 17 shows angiogenesis of the NOArg-PEA, Arg-PEA and NOArg-Arg co-PEA treated wound. (A) CD31 staining of wound tissue of day 7 and day 14; (B) blood vessel density in the wound tissue (n=3). Statistically significant, * p<0.05, ** p<0.01. From left to right the columns in each series are control, NOArg-PEA, 50/50-PEA, 20/80-PEA, and Arg-PEA.

FIG. 18 shows GPC chromatograms of NOArg-PEA, NOArg-Arg co-PEAs and Arg-PEA.

FIG. 19 shows live/dead staining of RAW 264.7 macrophages and 3T3 fibroblasts after 24 h treatment with 2 mg/mL 2-NOArg-4 PEA, 2-NOArg-4-Arg-4 50/50-PEA and 2-Arg-4 PEA.

FIG. 20 shows (A) chemical structure of an NOArg-Arg PEA copolymer repeating unit. x and y can range from 1 to 12. (B) show chemical structures of NOArg-Arg-Phe PEA copolymer repeating units.

FIG. 21 shows TEM images of the NOArg-Arg and NOArg-Arg-Phe PEA copolymers self-assembled particles in aqueous solution, (A) P5 and (B) P8.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). Illustrative examples of groups include:

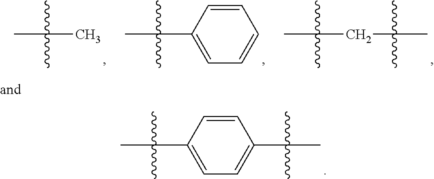

As used herein, unless otherwise indicated, the term “alkyl” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, the alkyl groups are a C₁ to C₃₀ alkyl group, including all integer numbers of carbons and ranges of numbers of carbons therebetween (e.g., C₁, C₂, C₃, C₄, C₅, C₆, C₇, C₉, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). The alkyl group may be unsubstituted or substituted with one or more substituent. Examples of substituents include, but are not limited to, substituents such as, for example, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

The present disclosure provides polymers, which includes copolymers and homopolymers (e.g., poly(ester amides) (PEAs)), methods of making same, and uses of same. The PEA polymers may be referred to as polymers. Copolymers may be referred to as polymers. The PEAs may be referred to as amino acid-based poly(esters) (AA-PEAs) or tunable immune-responsive (TIR) AA-PEAs. The polymers of the present disclosure may inhibit nitric oxide synthase in an individual.

This disclosure provides, in various examples, a new family platform of biodegradable synthetic polymeric biomaterials for providing tunable immune responsive capability via the inhibition of nitric oxide synthase (NOS). Such inhibition may facilitate the transition of the inflammatory response of wounds toward a faster and better wound healing, such as, for example, in challenging wound healing conditions like diabetic wounds. L-Arginine (Arg) is a common substrate for macrophages to metabolize into NOS and arginase. Both of these Arg metabolism pathways may participate in the wound healing process. Impaired wound healing, such as those associated with diabetic or other chronic wounds may be associated with an overproduction of NO by macrophages via their NOS pathway. Topical supplement of Arg may increase NO level of the wound, whereas NOS inhibitors, such as nitroarginine (NOArg) and its derivatives, may suppress NO production.

In various examples, a polymers or composition of the present disclosure exhibit one or both of:

(1) NOS inhibition (e.g., NOS inhibition relative to an untreated condition (e.g., wound)); and/or

(2) increased production by CAM of TGF-b1 (e.g., increased production by CAM of TGF-b1 relative to an untreated condition, such as, for example, a wound).

In an aspect, the present disclosure provides polymers. Polymers have at least one nitro guanidinium group. Polymers of the present disclosure may be made by methods described in the Examples provided herein.

A poly(ester amide) (PEA) polymer may comprise a first pendant group, where the first pendant group comprises an alkyl nitroguanidine, and at least a second pendant group chosen from an alkyl guanidine group and a side chain of a canonical amino acid, where the alkyl chain of the alkyl nitroguanidine group and alkyl guanidine group is 1 to 6 carbon atoms. In various examples, 10 to 90% of the pendant groups is alkyl nitroguanidine groups, including every 0.1% value and range therein. In various examples, the second pendant group is the side chain of arginine. In various other examples, the second or a third pendant group is the side chain of a hydrophobic amino acid or a cross-linking group.

In an example, the polymers of the present disclosure have the following structure:

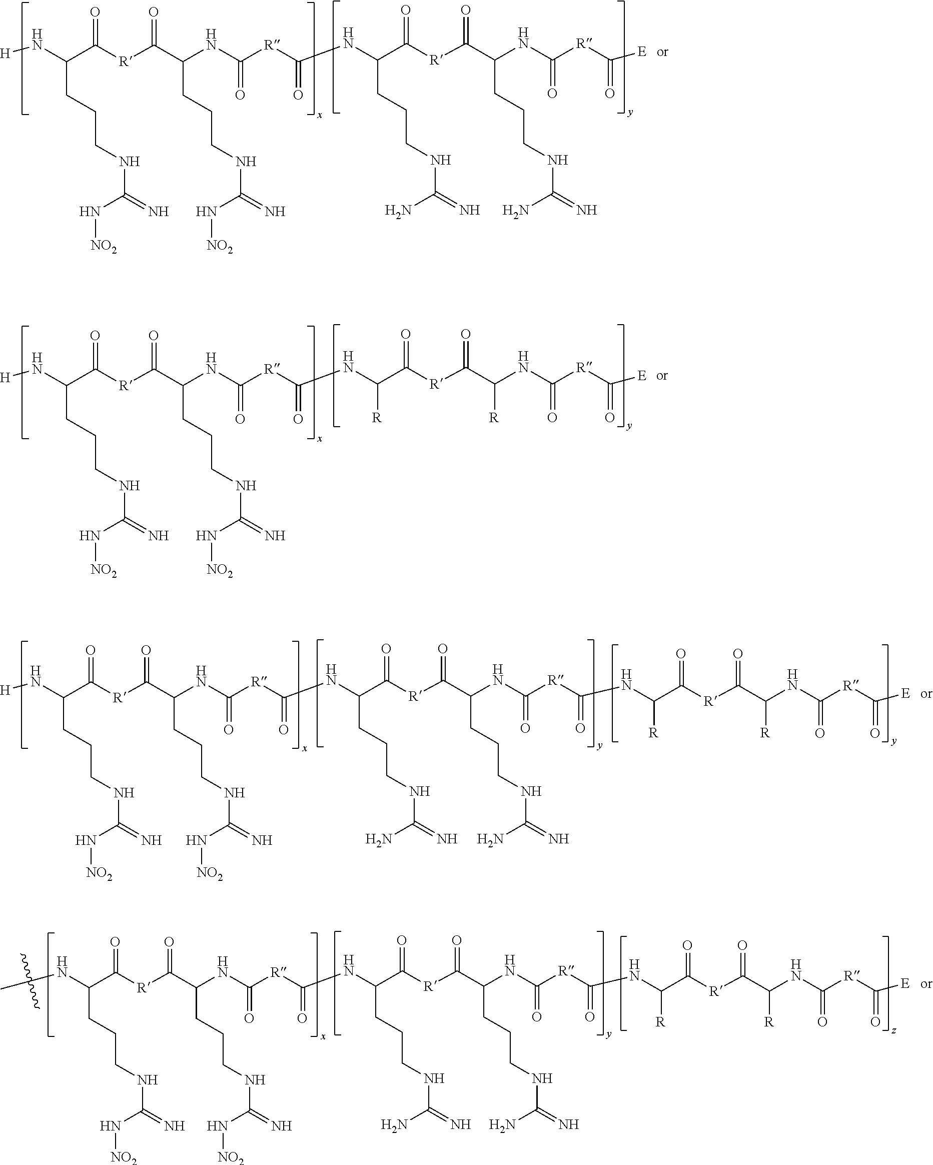

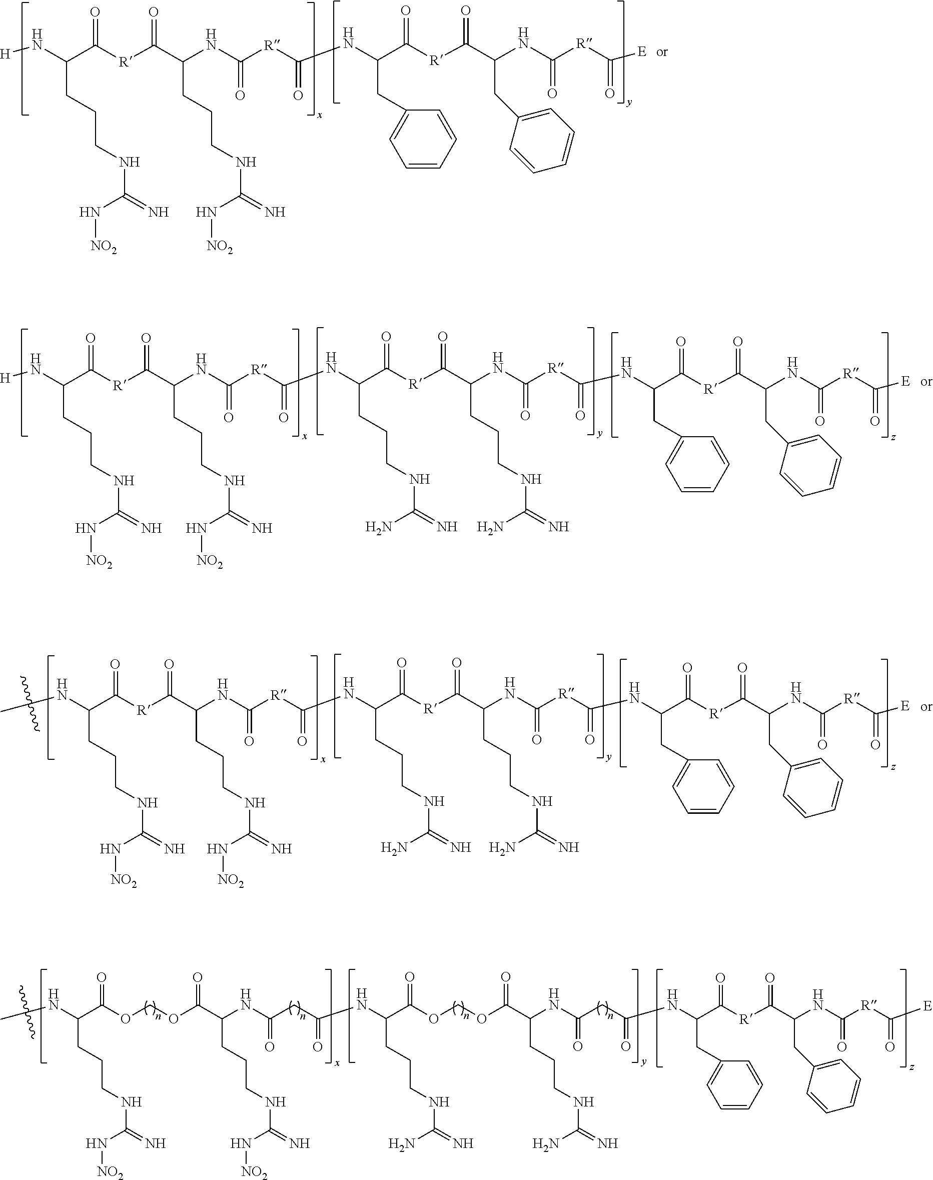

or a salt thereof,
where R at each occurrence is independently chosen from the side chain of a hydrophobic amino acid (e.g., Leu, Gly, Ala, Val, Ile, Pro, Met, Trp) and a cross-linking group (e.g., a vinyl group, alkyl vinyl group, and the like), R′ at each occurrence in the compound is independently chosen from

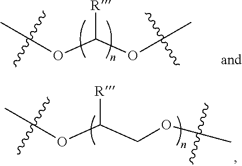

where R′″ at each occurrence in the compound is independently chosen from H and an alkyl group, and where n is 1-10 (1, 2, 3, 4, 5, 6, 7, 8, 9, 10), including all integer values and ranges therebetween (e.g., 1-4 (1, 2, 3, 4)). R″ at each occurrence in the polymer is independently chosen from

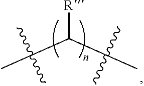

where R′″ at each occurrence in the polymer is independently chosen from H and an alkyl group, and n is 1-10 (1, 2, 3, 4, 5, 6, 7, 8, 9, 10), including all integer values and ranges therebetween (e.g., 1-4 (1, 2, 3, 4)). E is chosen from —H,

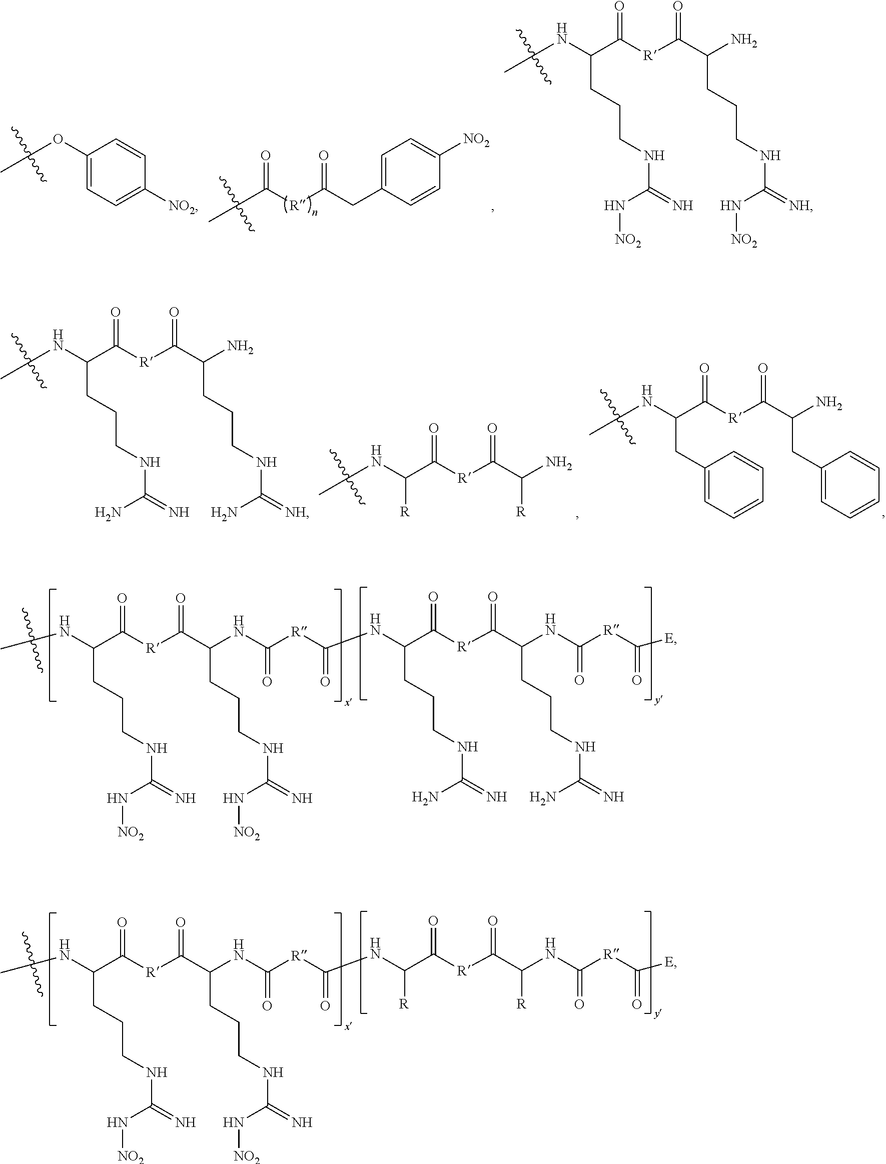

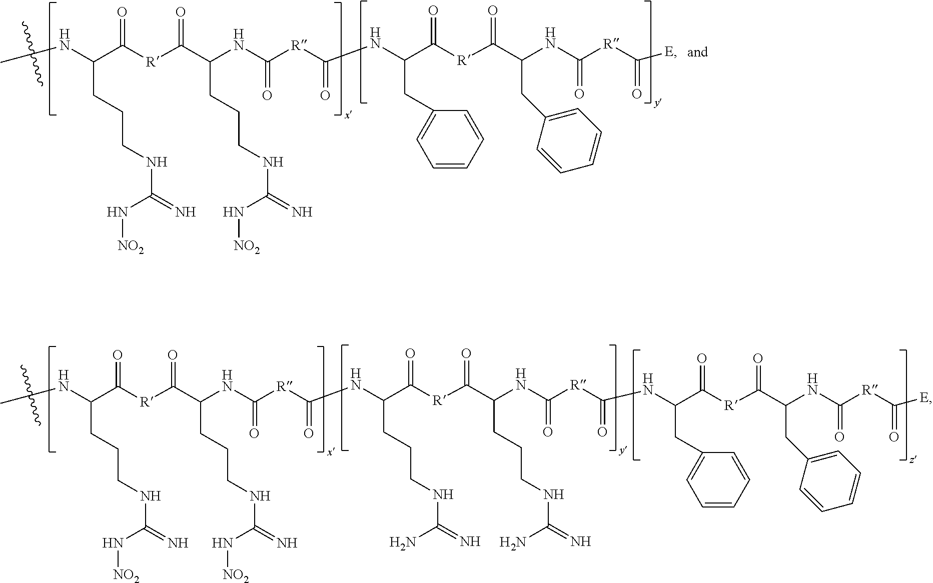

where R at each occurrence is independently chosen from the side chain of a hydrophobic amino acid and a cross-linking group (e.g., a vinyl group, alkyl vinyl group, and the like); R′ at each occurrence in the polymer is independently chosen from

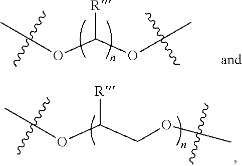

where R′″ at each occurrence in the polymer is independently chosen from H and an alkyl group, and where n is 1-10 (1, 2, 3, 4, 5, 6, 7, 8, 9, 10), including all integer values and ranges therebetween (e.g., 1-4 (1, 2, 3, 4)). R″ at each occurrence in the polymer is independently chosen from

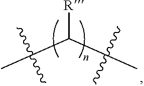

where R′″ at each occurrence in the polymer is independently chosen from H and an alkyl group, and n is 1-10 (1, 2, 3, 4, 5, 6, 7, 8, 9, 10), including all integer values and ranges therebetween (e.g., 1-4 (1, 2, 3, 4). x, y, z′, x′, y′, and z′ are independently at each occurrence 0-100, including all integer values and ranges therebetween. In such a polymer,

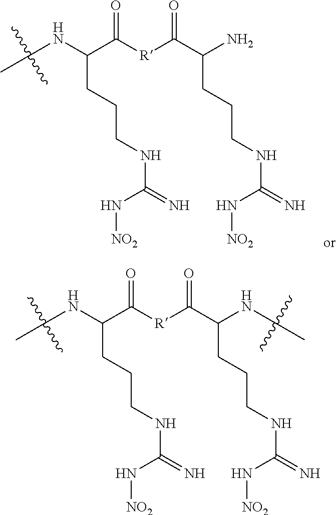

occurs at least once. The stereochemistry may be L or D.

In an example, a polymer of the present disclosure has the following structure:

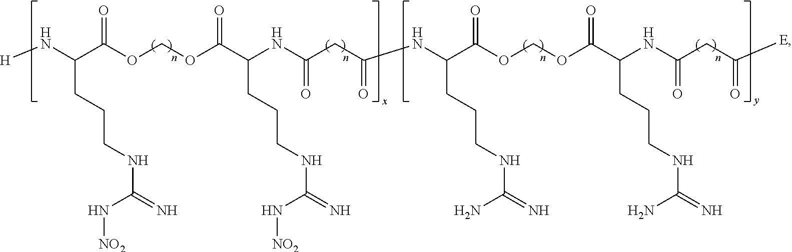

where E is independently at each occurrence chosen from —H,

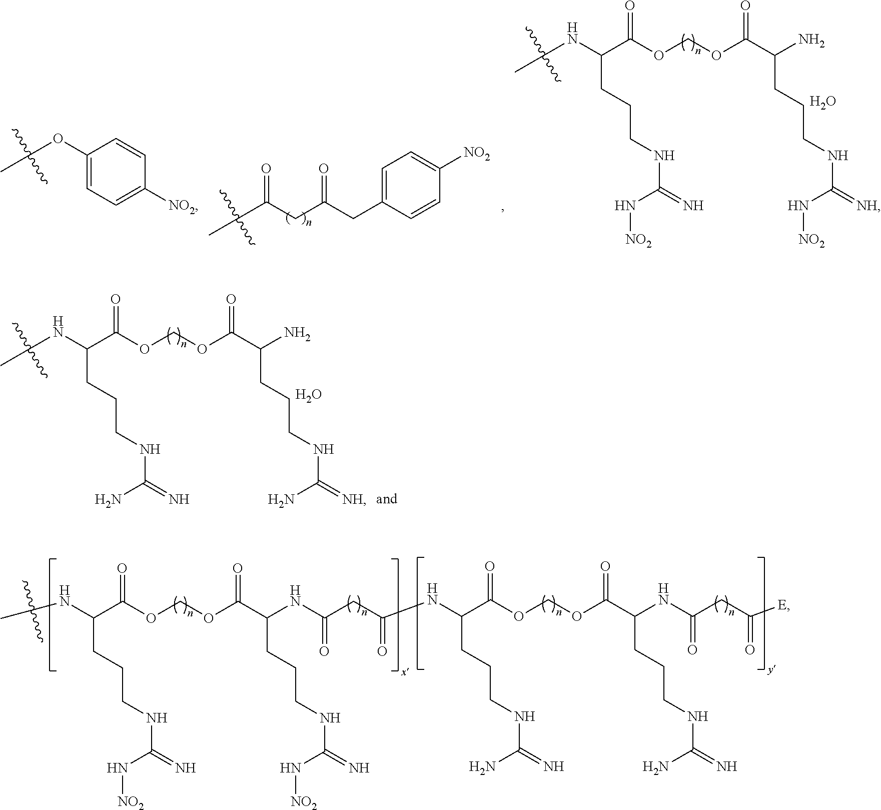

where x, y, x′, and y′ are independently at each occurrence 0-100, including all values and ranges therebetween, and n is independently at each occurrence 1-10, including all values and ranges therebetween (e.g., 1-4), with the proviso that

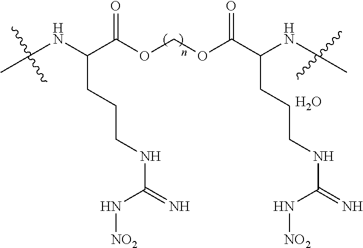

occurs at least once.

In an example, the polymers have one or more counter-ions (e.g., having a pKa from about −7 to +5) associated with positively charged groups (e.g., the alkyl guanidinium group of arginine) therein. Examples of counter-ions suitable to associate with the polymers are counter-anions of weak acids. Examples of such counter-anions include CH₃COO⁻, CF₃COO⁻, CCl₃COO⁻, Tos⁻ (Tos=p-toluene sulfonic acid, ester) and the like. Other examples of suitable counter ions include halides, such as F⁻, Cl⁻ and Br⁻, sulfate and nitrate. In one example, polymers have one or more ammonium groups that are present as a halide, Tos⁻, acetate, halogen-substituted acetate, sulfate, nitrate, or a combination thereof, salt.

Non-limiting examples of amino acids include naturally occurring amino acids. Naturally occurring amino acids include, but are not limited to, canonical amino acids.

A cross-linking group can react with another cross-linking group to cross-link the polymer. For example, a side chain having a reactive group has the following structure:

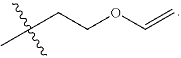 [SMILES: *CCOC=C]

For example, a side chain can be formed using a glycerin derivative. Various other examples of cross-linkable groups include:

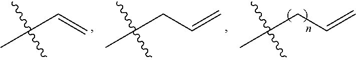

where n is 1-10, and the like. Other suitable cross-linking groups are known in the art.

A polymer of the present disclosure may comprise a plurality of blocks. In various examples, individual blocks comprise one or more of the following structures:

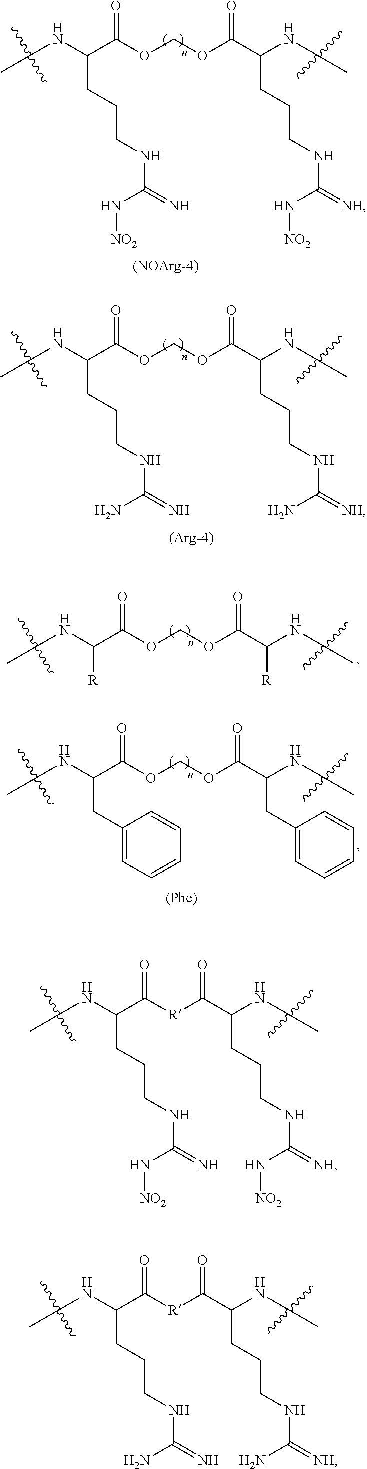

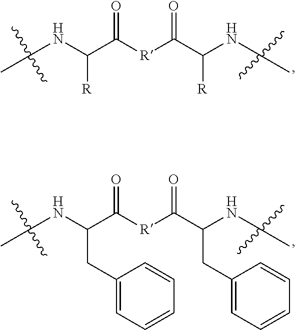

or a combinations thereof.

In an example, the ratio of the blocks of the polymer of the present disclosure (e.g., NOArg-4 to Arg-4) is 0.1-10: 0-10 (e.g., 1:0, 1:4, and 1:1), including all 0.1 ratio values and ranges therebetween. Other ratios include 4:6 to 6:4 or 5:5 to 1:9. In various examples, the ratio of Phe to NOArg-4 and Arg-4 is 1:10 to 5:10, , including all 0.1 ratio values and ranges therebetween, with the ratios of NOArg-4 to Arg-4 is 1:9 to 10:0, including all 0.1 ratio values and ranges therebetween.

The polymers of the present disclosure can have a range of number averaged molecular weights, Mₙ, and/or a weight averaged molecular weights, M_{w}. The polymers can have a Mₙ of from 0.4 kg/mol to 100 kg/mol, including all integers and ranges to the 0.1 kg/mol therebetween. For example, the polymers can have a M_{w} of from 0.4 kg/mol to 100 kg/mol, including all integers and ranges to the 0.1 kg/mol therebetween. The Mₙ and/or M_{w} of the polymers can be determined by, for example, gel permeation chromatography. Using M_{w} and Mₙ, the polydispersity index (PDI or Ð) can be determined. In various examples, a polymer of the present disclosure has a number average molecular weight (Mₙ) of 10,000 to 50,000 g/mol, including all g/mol values and ranges therebetween (e.g., 15,000-25,000 g/mol) and/or a weight average molecular weight (M_{w}) of 10,000 to 50,000, including all g/mol values and ranges therebetween (e.g., 25,000-35,000 g/mol). In an example, a polymer of the present disclosure has a PDI of 1.2 to 1.5, including all 0.01 values and ranges therebetween (e.g., 1.35-1.50).

Polymers of the present disclosure may be biocompatible and biodegradable.

In an aspect, the present disclosure provides compositions. Compositions of the present disclosure comprise at least one polymer of the present disclosure and an acceptable pharmaceutical carrier.

Pharmaceutically acceptable carriers are determined in part by the particular composition being administered, as well as by the particular method used to administer the composition. Accordingly, there are a wide variety of suitable formulations of pharmaceutical compositions of the present disclosure (see, e.g., Remington's Pharmaceutical Sciences, 20^{th} ed., 2003, supra). Effective formulations include topical formulations (e.g., topical formulations formulated for with extended release) and the like.

The compositions may comprise polymers formulated into nanoparticles, films, gels (e.g., hydrogels), fibrous membranes, nanofibers, liposomes, micelles and the like, and combinations thereof. The nanoparticles may be self-assembled nanovesicles comprising copolymers of the present disclosure.

For example, a nanovesicles may be formed by adjusting the number of methylene groups in Arg diester monomers, NO-Arg-based diester monomers, and dicarboxylic acid monomers, such that copolymers formed from the monomers assemble into nanoparticles. In various other examples, a third monomer comprising a hydrophobic amino acid-based diester (e.g., a Phe-based diester) or cross-linking group-based diester (e.g., a vinyl group-based diester) is polymerized with Arg and NO-Arg-based monomers diesters resulting in copolymer formed from the Arg-based monomer, the NO-Arg-based monomer, and hydrophobic amino acid-based monomer. Hydrophobic amino acids include, but are not limited to, leucine, isoleucine, phenylalanine, valine, tryptophan, glycine, alanine, proline, methionine, and the like (e.g., hydrophobic non-canonical amino acids). In various examples, cysteine may be used to form disulfide bonds between polymers. The stereochemistry of the monomers may be L or D. Monomers may have cross-linking groups, such as, for example, vinyl groups, alkyl vinyl groups, and the like. Cross-linking groups may be used to form polymers having a gel or gel-like state (e.g., hydrogel).

For example, a nanoparticle is formed by dissolving a copolymer (e.g., a copolymer formed from at least three monomers, such as, for example, an Arg diester monomer, an NO-Arg diester monomer, and a hydrophobic amino acid-based diester monomer or a cross-linking group-based diester monomer) or a copolymer formed from Arg and NO-Arg-based diester monomers having an x and/or y value of 1 to 12 (e.g., 1, 2, 3, 4, 5, 6, 7, 8, 9, 10, 11, or 12)) in a solvent (e.g., DMSO) at a concentration of 0.1 to 15 mg/mL, including every 0.01 mg/mL value and range there between (e.g., 0.1 to 10 mg/mL, 1 to 10 mg/mL, 1 to 15 mg/mL, 0.5 mg/mL). The dissolved copolymers are dialyzed against water (e.g., deionized water) for a length of time (e.g., 0.5 to 20 hours, including every minute value and range there between (e.g., 6 to 24 hours, 24 hours)). The copolymer solution is collected after dialysis and the solution comprises the nanoparticles comprising the copolymer. Various ratios of monomers may be used to form the copolymer. The copolymer solution may be concentrated by various means known in the art, such as, for example, by lyophilization and then resuspended in aqueous media (e.g., water at 0 to 20° C. or room temperature) at a concentration of 1 to 10 mg/mL, including every mg/mL value and range therebetween.

Compositions may comprise a plurality of nanoparticles comprising a copolymer or a combination of copolymers of the present disclosure. The nanoparticles may have a size (e.g., a longest linear dimension, which may be a diameter) of 30 to 1000 nm, including every nanometer value and range therebetween (e.g., 30 to 500 nm, 30 to 40 nm). The plurality of nanoparticles may comprise nanoparticles of the same size, substantially the same size, similar size, or different sizes. For example, in a plurality of nanoparticles may comprise nanoparticles with sizes (e.g., longest linear dimensions, which may be diameters) of 30 to 40 nm, including every nanometer value and range therebetween. In various other examples, a plurality of nanoparticles may comprise nanoparticles with sizes of 30 to 1000 nm including every nanometer value and range therebetween.

In an aspect, the present disclosure provides methods of using the polymers and/or compositions of the present disclosure. The polymers and/or compositions can be used, for example, in methods of treatment.

Compositions of the present disclosure also include extended-release formulations. In some embodiments, extended-release formulations useful in the present disclosure are described in U.S. Pat. No. 6,699,508, which can be prepared according to U.S. Pat. No. 7,125,567, the disclosures of which with respect to extended-release formulations are incorporated herein by reference.

The pharmaceutical compositions can be administered to the patient in a variety of ways, including, for example, but not limited to, topically and the like.

Compositions of the present disclosure can be administered as frequently as necessary, including hourly, daily, weekly or monthly. The dosages may be varied depending upon the requirements of the patient, the severity of the condition (e.g., wound, inflammation, disease, and the like) being treated, and the polymer or composition being employed. For example, dosages can be empirically determined considering the type and stage of condition (e.g., wound, inflammation) in a particular patient. The dose administered to a patient, in the context of the present disclosure, should be sufficient to effect a beneficial therapeutic response (e.g., wound healing, reduction in inflammation) in the patient over time. Determination of the proper dosage for a particular situation is within the skill of the practitioner.

In various examples, the present disclosure provides a method of treating a condition (e.g., inflammation and conditions and diseases associated with inflammation), including administering to a subject in need of such treatment a therapeutically effective amount of a composition or polymer of the present disclosure. The polymers and/or compositions may be used to treat diseases associated with, caused by, and/or causing inflammation. Examples of diseases and conditions include, but are not limited to, diabetes, allergies, asthma, atherosclerosis, Crohn's disease, arthritis, obesity, and the like, and combinations thereof. A subject in need of treatment may be in need of treatment for various injuries, various disorders, various wounds, and various diseases.

The compositions or polymers of the present disclosure can be administered to a subject in need of treatment (e.g., in need of treatment for inflammation, a disease and/or condition associated with inflammation, a wound, such as, for example, a diabetic wound, a pressure wound, inflammation, and the like).

A subject in need of treatment may be a human or non-human mammal. Non-limiting examples of non-human mammals include cows, pigs, mice, rats, rabbits, cats, dogs, other agricultural animal, pet, service animals, and the like.

In various examples, compositions and/or polymers of the present disclosure can be used to treat wounds and/or treat inflammation. Wounds and/or inflammation can be caused by, for example, diabetes (e.g., obesity-induced diabetes), excess weight, and the like, and combinations thereof.

Compositions and/or polymers of the present disclosure may increase the rate of wound healing. A composition and/or polymer may increase the rate a wound may heal 40-81% (e.g., 43-81%) faster than a wound (e.g., substantially similar wound) treated with a 100% Arg-based PEA polymer. In various examples, the wound is healed 40%, 50%, 60%, 70%, or 80% faster. A composition and/or polymer may increase the rate a wound may heal 5-30% faster than a wound (e.g., substantially similar wound) treated with a 100% NO-Arg-based PEA polymer. In various examples, the wound is healed 5%, 6%, 7%, 8%, 9%, 10%, 20%, 25%, or 30% faster.

The polymers and/or compositions of the present disclosure may be used to decrease or increase inflammation. For example, the amount of NO-Arg in a polymer (e.g., a composition comprising a polymer) can be used to tune the anti-inflammatory or inflammatory effect of polymer. In such an example, increasing the amount of NO-Arg in a polymer increases the anti-inflammatory effects of a polymer of the present disclosure.

The steps of any of the methods described in the various examples disclosed herein are sufficient to carry out the methods and produce the compositions of the present disclosure. Thus, in an example, the method consists essentially of a combination of the steps of the methods disclosed herein. In another embodiment, the method consists of such steps.

In an aspect, the disclosure provides kits. A kit may comprise pharmaceutical preparations containing any one or any combination of polymers of the present disclosure and printed material. In an example, a kit comprises a closed or sealed package that contains the pharmaceutical preparation. In various examples, the package comprises one or more closed or sealed vials, bottles, blister (bubble) packs, or any other suitable packaging for the sale, or distribution, or use of the polymers and compositions comprising polymers of the present disclosure. The printed material may include printed information. The printed information may be provided on a label, or on a paper insert, or printed on the packaging material itself. The printed information may include information that identifies the polymers in the package, the amounts and types of other active and/or inactive ingredients, and instructions for taking the composition, such as the number of doses to take over a given period of time, and/or information directed to a pharmacist and/or another health care provider, such as a physician, or a patient. The printed material may include an indication that the pharmaceutical composition and/or any other agent provided with it is for treatment of a subject having one or more wound and/or inflammation. In various examples, the product includes a label describing the contents of the container and providing indications and/or instructions regarding use of the contents of the container to treat a subject having a wound (e.g., a wound caused by diabetes and/or excess weight). A kit may comprise a single dose or multiple doses.

In an aspect, the present disclosure provides articles of manufacture. The articles of manufacture may comprise polymers and compositions of the present disclosure. For example, an article of manufacture is a medical device, such as, for example, a bandage, a wound dressing, wound care tool, films, and the like.

In the following Statements, various examples of the methods and compositions of the present disclosure are described:

Statement 1. A poly(ester amide) (PEA) polymer, comprising a first pendant group, where the first pendant group comprises an alkyl nitroguanidine, and at least a second pendant group chosen from an alkyl guanidine group and a side chain of a canonical amino acid, where the alkyl chain of the alkyl nitroguanidine group and alkyl guanidine group is 1 to 6 carbon atoms.
Statement 2. The PEA polymer according to Statement 1, where 10 to 90% of the pendant groups are alkyl nitroguanidine groups.
Statement 3. The PEA polymer according to Statement 1 or Statement 2, where the second pendant group is the side chain of arginine.
Statement 4. The PEA polymer according to any one of the preceding Statements where the polymer is formulated as a gel, nanoparticle, nanofiber, liposome, micelle, or a combination thereof.
Statement 5. The PEA polymer according to any one of the preceding Statements, where the polymer is biocompatible.
Statement 6. The PEA polymer according to any one of the preceding Statements, where the polymer is biodegradable.
Statement 7. The PEA polymer according to any one of the preceding Statements having the following structure:

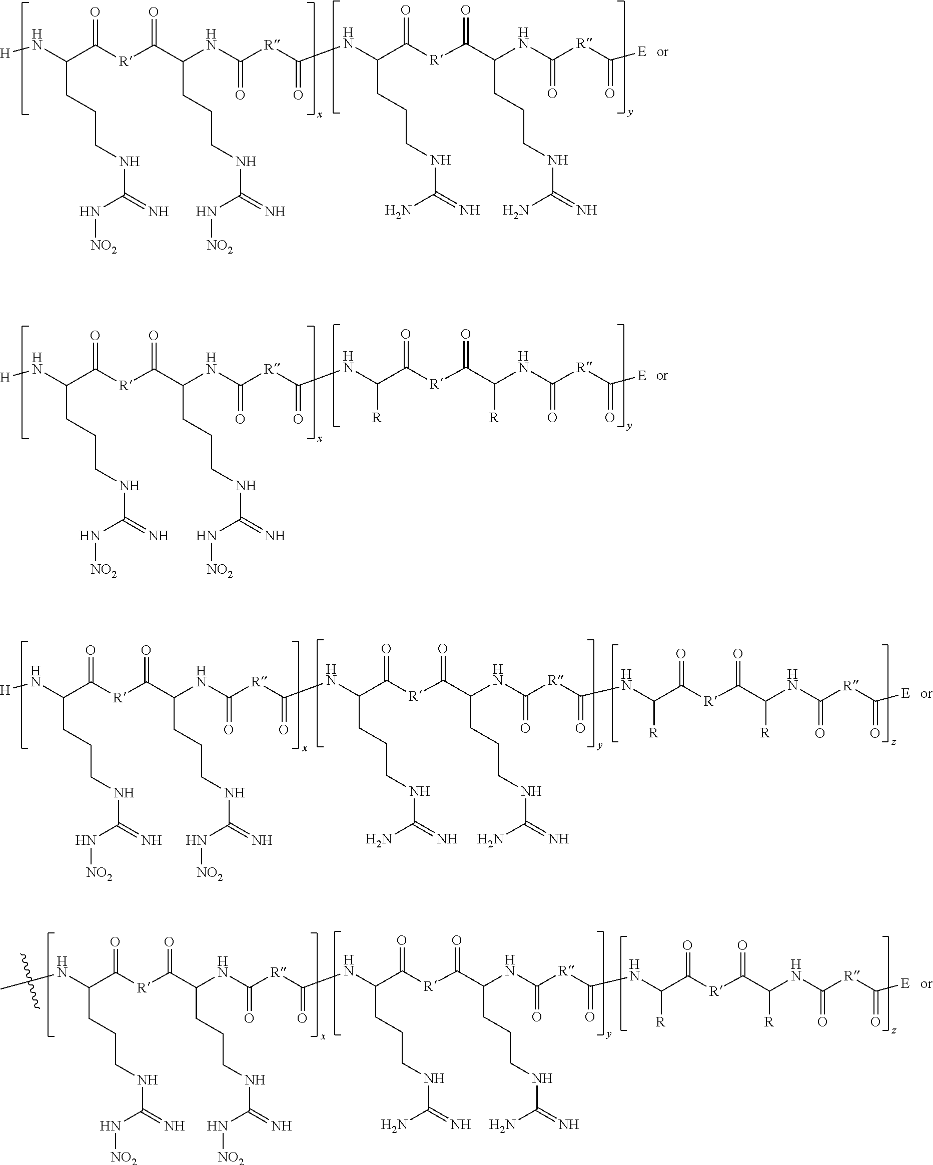

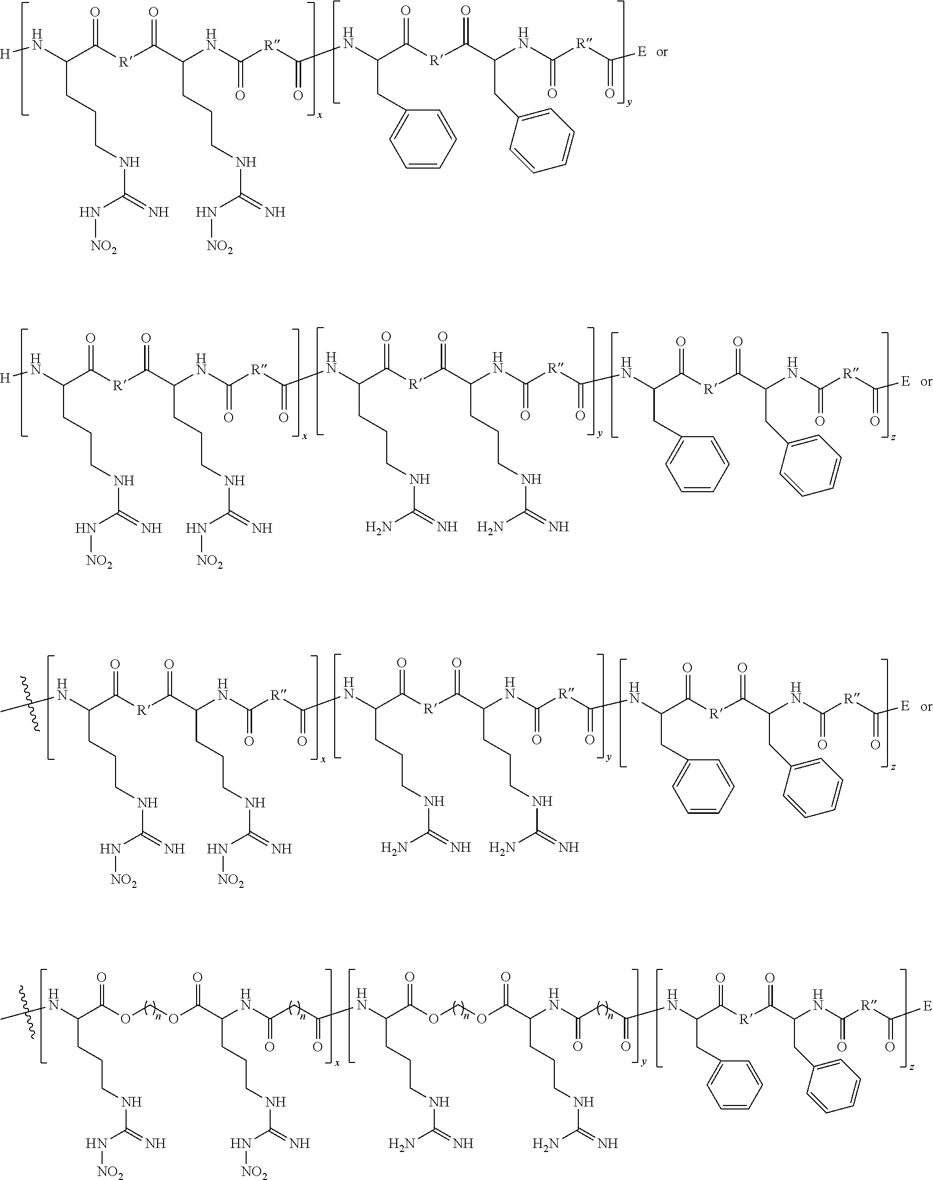

or a salt thereof,
where

where R at each occurrence is independently chosen from the side chain of a hydrophobic amino acid and a cross-linking group (e.g., a vinyl group, alkyl vinyl group, and the like);

R′ at each occurrence in the polymer is independently chosen from

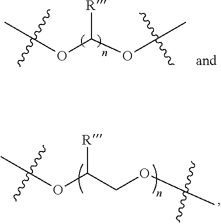

where R′″ at each occurrence in the polymer is independently chosen from H and an alkyl group, and where n is 1-10 including all integer values and ranges therebetween (e.g., 1-4);

R″ at each occurrence in the polymer is independently chosen from

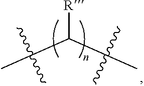

where R′″ at each occurrence in the polymer is independently chosen from H and an alkyl group, and n is 1-10, including all integer values and ranges therebetween (e.g., 1-4);

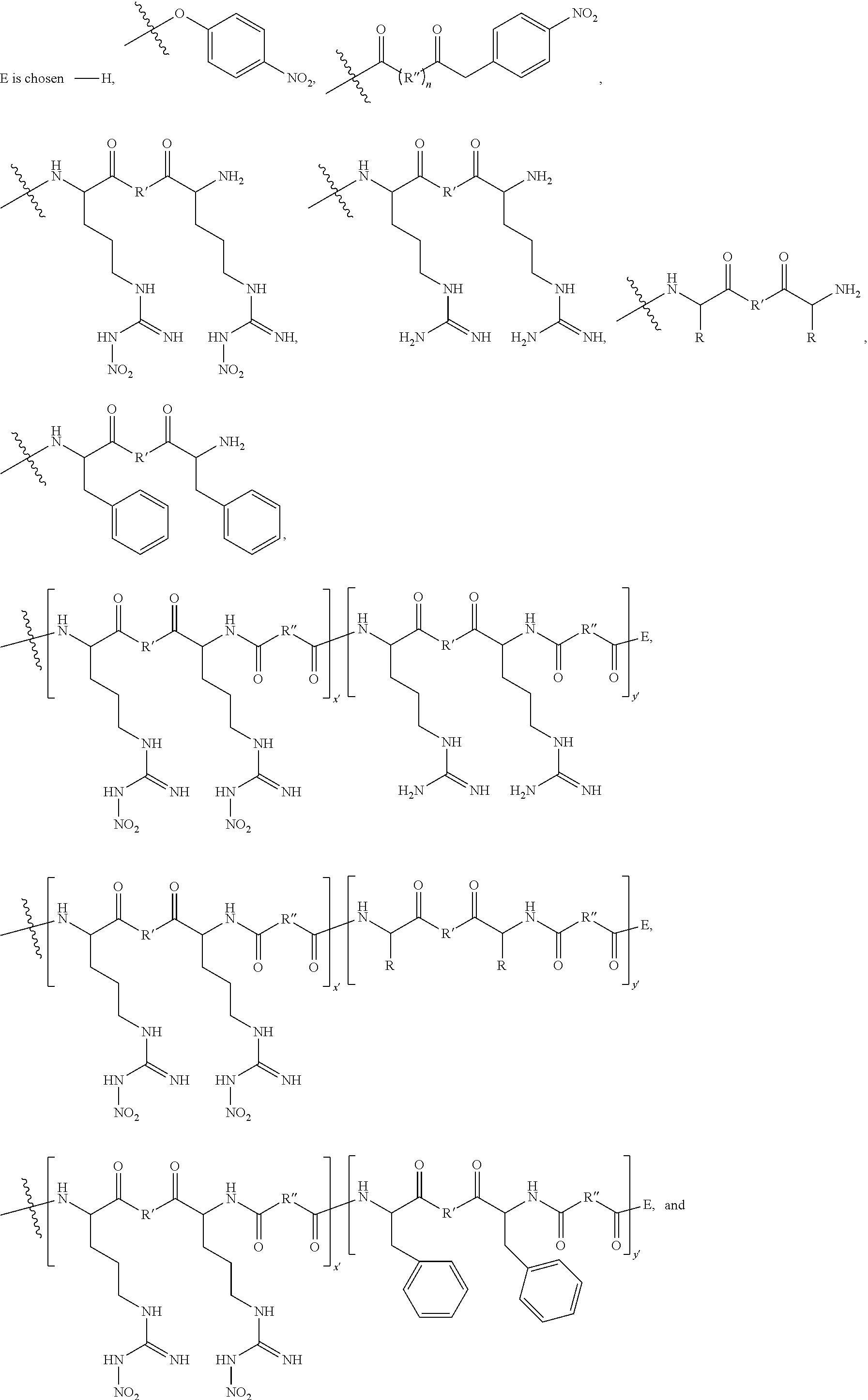

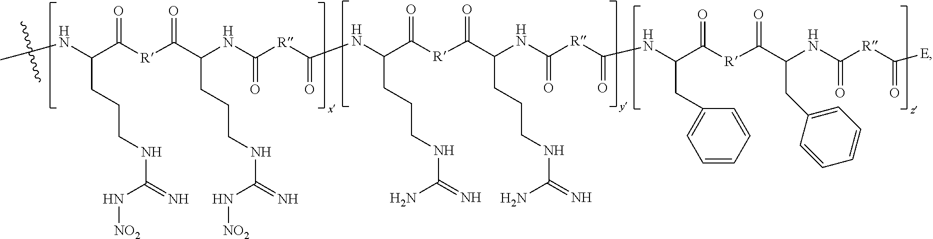

where R at each occurrence is independently chosen from the side chain of a hydrophobic amino acid and a cross-linking group (e.g., a vinyl group, alkyl vinyl group, and the like); R′ at each occurrence in the polymer is independently chosen from

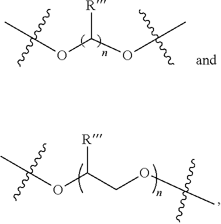

where R′″ at each occurrence in the polymer is independently chosen from H and an alkyl group, and wherein n is 1-10, including all integer values and ranges therebetween (e.g., 1-4);

R″ at each occurrence in the polymer is independently chosen from

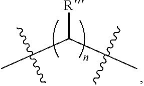

where R′″ at each occurrence in the polymer is independently chosen from H and an alkyl group, and n is 1-10, including all integer values and ranges therebetween (e.g., 1-4)

x, y, z, x′, y′, and z′ are independently at each occurrence 0-100, including all integer values and ranges therebetween;

with the proviso:

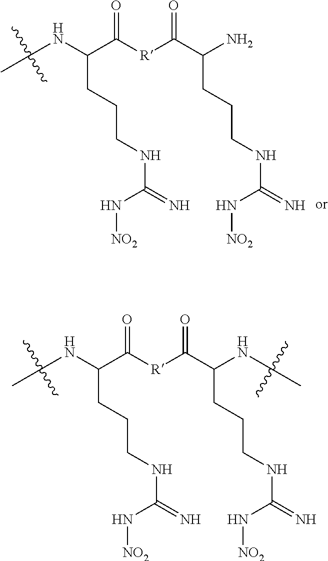

occurs at least once.
Statement 8. The PEA polymer according to any one of the preceding Statements, where the polymer has the following structure:

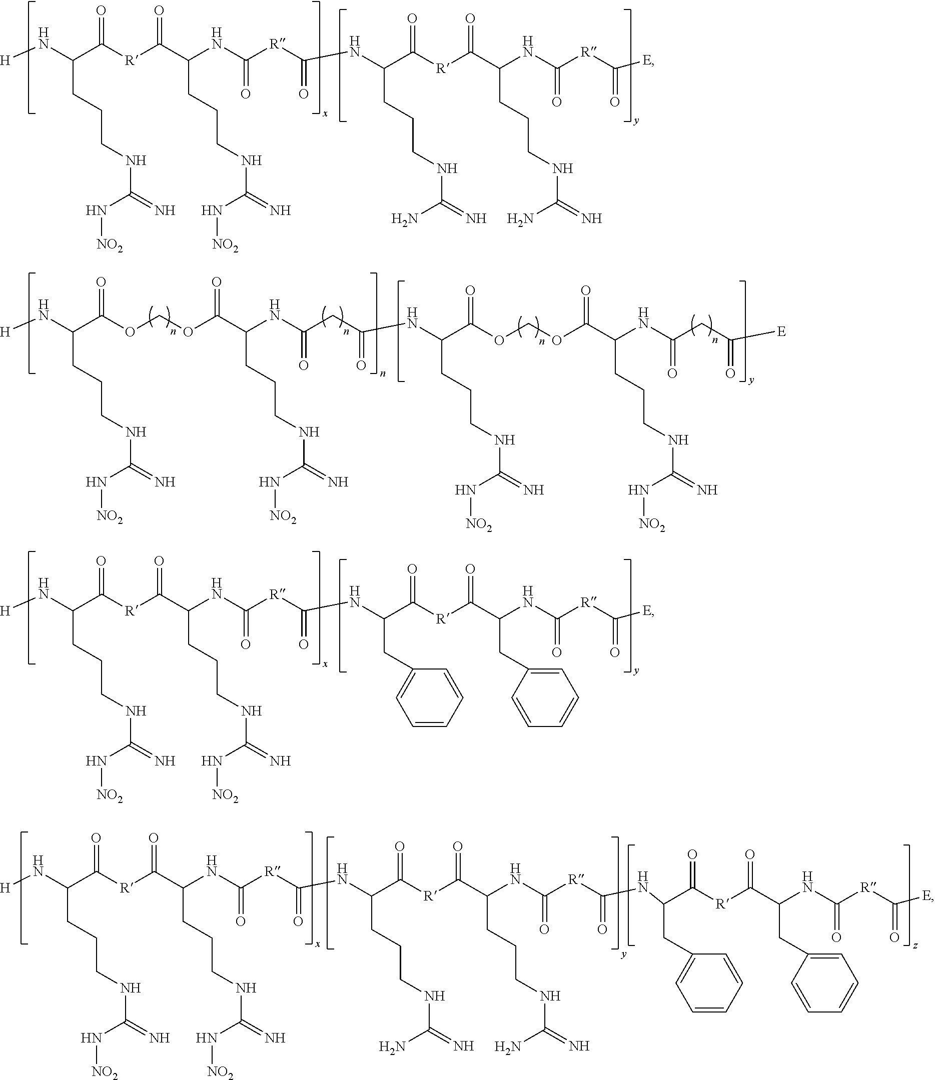

salts thereof, or a combination thereof,
where E is independently chosen from —H,

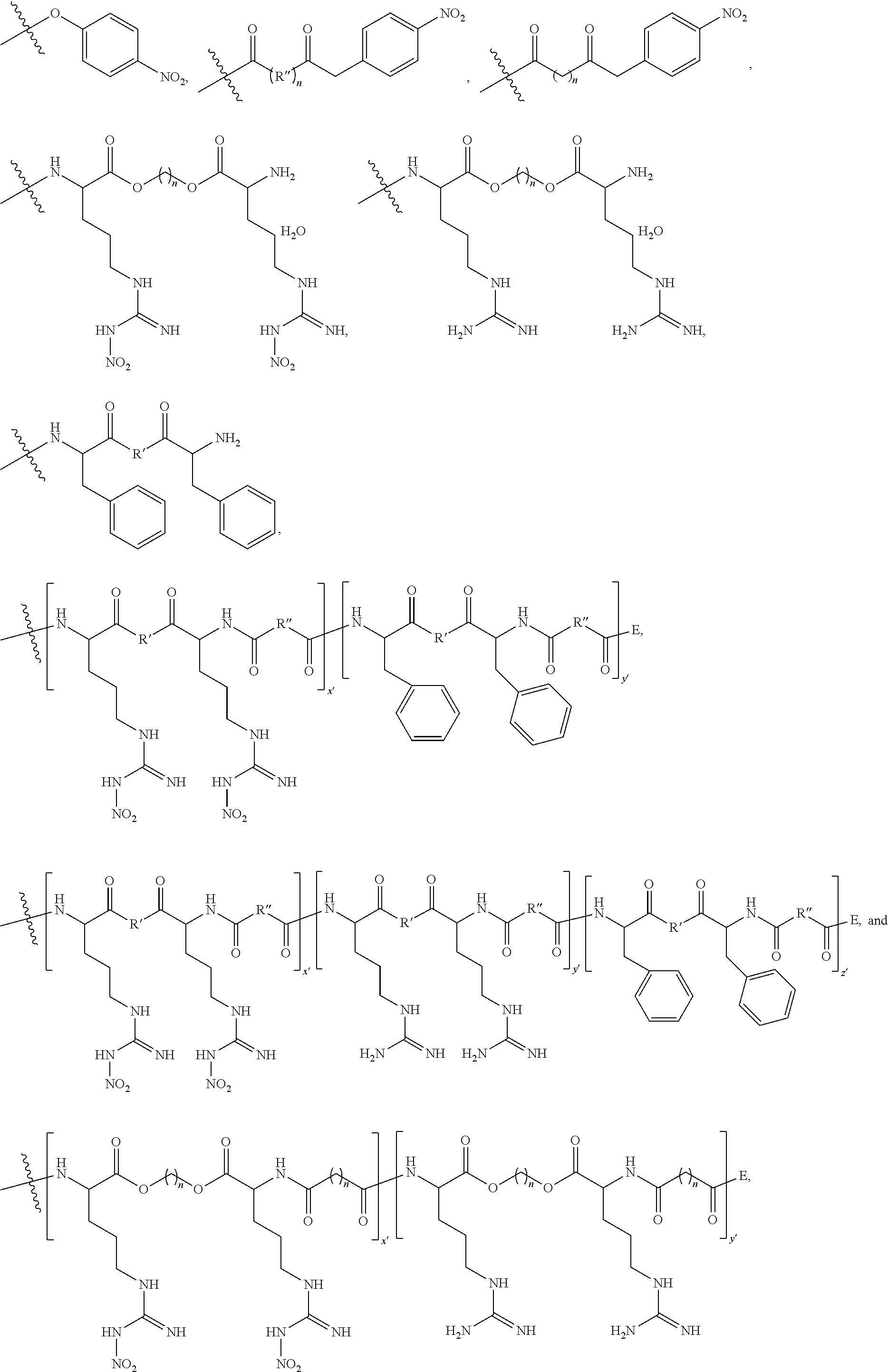

where x, y, z, x′, y′, and z′ are independently at each occurrence 0-100, including all values and ranges therebetween, and n is independently at each occurrence 0-10 (e.g., 0-4), including all values and ranges therebetween, with the proviso that

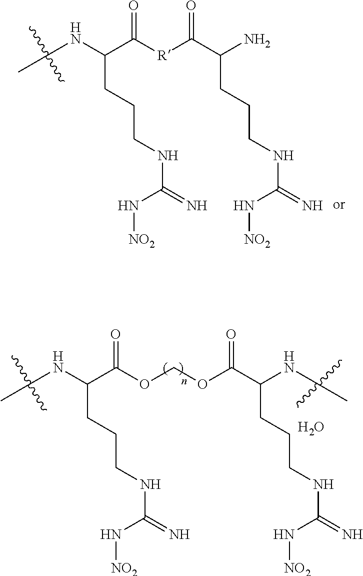

occurs at least once.
Statement 9. The PEA polymer according to any one of the preceding Statements, where the ratio of

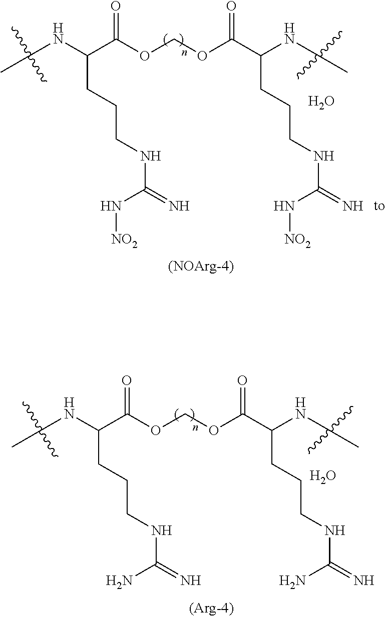

is 1:0, where n is 4.
Statement 10. The PEA polymer according to any one of the preceding Statements, where the ratio of NOArg-4 to Arg-4 is 0.1-10:0-10.
Statement 11. The PEA polymer according to Statement 10, where the ratio of NOArg-4 to Arg-4 is 1:4.
Statement 12. The PEA polymer according to Statement 10, where the ratio of NOArg-4 to Arg-4 is 1:1.
Statement 13. The PEA polymer according to any one of Statements 1-8, where the ratio of

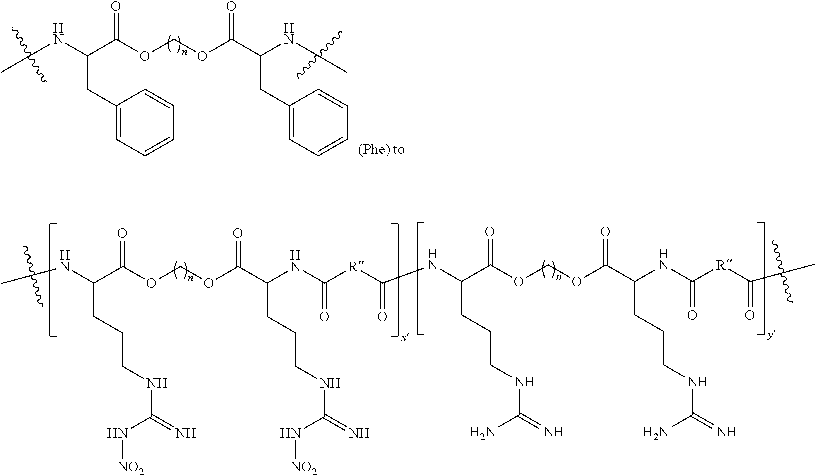

is 1:10 to 5:10 and the ratio of NOArg-4 to Arg-4 is 1:9 to 10:0, where n is 4.
Statement 14. The PEA polymer according to any one of the preceding Statements, wherein the number average molecular weight (Mₙ) is 10,000-50,000 g/mol, including all g/mol values and ranges therebetween.
Statement 15. The PEA polymer according to any one of the preceding Statements, wherein the weight average molecular weight (M_{w}) is 10,000-50,000 g/mol, including all g/mol values and ranges therebetween.
Statement 16. The PEA polymer according to any one of the preceding Statements, wherein the PDI is 1.20-1.50, including all 0.01 values and ranges therebetween.
Statement 17. A composition comprising a PEA polymer according to any one of the preceding Statements and a pharmaceutically acceptable carrier.
Statement 18. The composition according to Statement 17, wherein the polymer is formulated as a gel, nanoparticle, nanofiber, liposome, micelle, or a combination thereof and is biocompatible and biodegradable.
Statement 19. A method of treating a subject in need of treatment with one or more PEA polymer according to any one of Statements 1-16 and/or one or more composition according to Statement 17 or Statement 18, comprising administering to the subject in need of treatment the PEA polymer according to any one of Statements 1-16 and/or one or more composition according to Statement 17 or Statement 18 (e.g., an effective amount of the PEA polymer according to any one of Statements 1-16 and/or one or more composition according to Statement 17 or Statement 18).
Statement 20. The method according to Statement 19, where the subject in need of treatment has one or more wound.
Statement 21. The method according to Statement 20, where the one or more wound is a diabetic wound, obesity associated wound, and/or pressure wound.
Statement 22. The method according to Statement 20 or Statement 21, where the one or more wound is healed 40-81% (e.g., 43-81%) faster than a wound (e.g., a substantially similar wound) treated with a 100% Arg-based PEA polymer.
Statement 23. The method according to Statement 22, where the one or more wound is healed at least 40% faster.
Statement 24. The method according to Statement 22, where the one or more wound is healed at least 70% faster.
Statement 25. The method according to Statement 18, where the one or more wound is healed 5-30% faster than one or more wound treated with a PEA polymer comprising a 100% NO-Arg-based PEA polymer.
Statement 26. The method according to Statement 25, where the one or more wound is healed at least 10% faster.
Statement 27. The method according to Statement 25, where the wound is healed at least 25% faster.
Statement 28. The method according to any one of Statements 19-27, wherein the subject in need of treatment has or is suspected of having a disease, injury, and/or disorder with inflammation character or inflammation as a symptom or cause.
Statement 29. The method according to any one of Statements 19-28, where wound healing or inflammation reduction is assessed by histology (e.g., hematoxylin and eosin staining) and/or immunohistochemistry (e.g., diaminobenzidine visualization).
Statement 30. The method according to any one of Statements 19-29, where the subject does not experience detectable cytotoxic effects (e.g., cytotoxic effects against the subject's macrophages and fibroblasts).
Statement 31. The method according to Statement 30, where the subject does not experience detectable cytotoxic effects.
Statement 32. A method of altering the anti-inflammatory or inflammatory response of a subject, comprising administering or applying a PEA polymer according to any one of Statements 1-16 and/or a composition according to Statement 17 or Statement 18 to the subject, where the anti-inflammatory or inflammatory response of the subject is altered by selecting the stoichiometry of alkyl nitroguanidine pendant group relative to the other pendant groups of the PEA polymer.
Statement 33. The method according to Statement 32, where the subject is diagnosed with or is suspected of having a disease, where the disease elicits or is caused by inflammation or the disease has inflammation symptoms or character.
Statement 34. The method according to Statement 32 or Statement 33, where the subject has a wound.
Statement 35. The method according to any one of Statements 32-34, where the subject has inflammation.
Statement 36. An article of manufacture comprising a PEA polymer according to any one of Statements 1-16.
Statement 37. The article of manufacture according to Statement 36, where the article of manufacture is a wound dressing, a bandage, or a wound care tool.

The following examples are presented to illustrate the present disclosure. They are not intended to be limiting in any matter.

### Example 1

This example provides a description of polymers of the present disclosure and uses thereof.

In this disclosure, a new family of water-soluble and biodegradable NOArg based polyester amide (NOArg-PEA) homopolymer and NOArg-Arg PEA copolymers has been designed and synthesized with different NOArg to Arg composition ratio for the purpose of actively facilitating the timely transition from a pro-inflammatory to a pro-healing wound microenvironment for an eventual treatment option for impaired wound healing. NOArg based PEA and NOArg-Arg co-PEAs are biodegradable (more than 50% degradation in vitro in 4 days at 37° C.), biocompatible and did not activate resting macrophage immune response per se. When NOArg-PEA and NOArg-Arg co-PEAs are used to treat classically activated macrophages (CAM) and alternatively activated macrophages (AAM), the NO production, TNF-α, arginase activity and TGF-β1 production from macrophage were altered and tuned. In general, NOArg-PEA and NOArg-Arg co-PEAs decreased the NO production of CAM, increased the arginase activity in both CAM and AAM, increased TGF-β1 production of CAM to various degrees and had no significant effect on TNF-α production of CAM and AAM. The in vitro results implied the NOArg-PEA and NOArg-Arg co-PEAs may timely facilitate the transition of wounds from pro-inflammatory status to anti-inflammatory status. Diabetic rat models were used to evaluate the effect of NOArg-PEA and NOArg-Arg co-PEAs on an impaired wound healing model. Diabetic rats treated with 2-NOArg-4 PEA, 2-NOArg-4-Arg-4 20/80, and 2-NOArg-4-Arg-4 50/50 achieved 44-81% faster wound healing capability compared with the untreated animal control on day 7. The data from the histological and immunohistochemical analysis showed that the 2-NOArg-4-Arg-4 20/80 and 2-NOArg-4-Arg-4 50/50 treatments led to a more M2 macrophage phenotype (CD206) and arginase I production than the control, i.e., suggesting pro-healing wound microenvironment with improved re-epithelialization during the first 7 days. Similar trend was retained in Day 14. The 2-NOArg-4-Arg-4 20/80 and 2-NOArg-4-Arg-4 50/50 treatments also increased the collagen deposition in the healing wound. This study showed that the NOArg-Arg co-PEA biomaterials have the potential as an advanced alternative for treating impaired wound healing, such as diabetic, chronic or burn wounds.

Experimental.

Materials.

L-Arginine hydrochloride (L-Arg-Cl) (Alfa Aesar, Ward Hill, Mass.), NOArg (Chem-impex int'l inc, IL), 1,4-butanediol (Alfa Aesar, Ward Hill, Mass.), triethylamine (TEA) (99%, EMD Chemical, Darmstadt, Germany), sodium nitrate (VWR Science, West Chester, Pa.), p-toluene sulfonic acid monohydrate (TsOH.H₂O) and p-nitrophenol (JT Baker, Philipsburg, N.J.) were used for the polymer synthesis. Solvents including N, N-dimethylacetamide (DMAc) (Beantown Chemical, Hudson, N.H.), isopropyl alcohol (ACS, 99.5%, Macron Chemicals, Philipsburg, N.J.), acetone, dimethyl sulfoxide (DMSO) (Mallinckrodt, St. Louis, Mo.), ethyl acetate (BDH, London, UK), ethanol, benzene, toluene and chloroform (VWR Science, West Chester, Pa.) were used without further purification. Molecular weight cutoff (MWCO) 1,000 g/mol and 3,500 g/mol snakeskin dialysis tubes were purchased from Thermo Fisher Scientific, Rockford, Ill. Bromine (VWR Science, West Chester, Pa.), NaOH (Macron Chemicals, Philipsburg, N.J.), α-naphthol (Beantown Chemical, Hudson, N.H.), urea (Fisher Scientific, Rockford, Ill.), α-isonitrosopropiophenone (Acros organics, Geel, Belgium) and Griess reagent (Enzo Life Science, Farmingdale, N.Y.) were used in Sakaguchi test, NO production and arginase activity assays. L-NAME was purchase from Alfa Aesar, Ward Hill, Mass.

Synthesis of Dihydrochloride acid salt of Bis (NOArg) butane diester monomers (NOArg-4) and Bis (Arg) butane diester monomers (Arg-4-CL).

The NOArg-4 monomer was synthesized using a previously described but modified method (i.e., in refluxing benzene instead of toluene). Briefly, NOArg (0.04 mol), 1,4 butanediol (0.018 mol), TsOH.H₂O (0.05 mol) and sodium nitrate (15 gram) were mixed well in 25 mL water in a flask. 150 mL benzene (b.p. 80° C.) was added and the heterogeneous solid-liquid reaction mixture was heated in an oil bath (87-90° C.) and refluxed for 36 hour (h). All water in the reaction was collected by a dean-stark apparatus. After the reaction was completed and cooled to room temperature, benzene was decanted. The product was dissolved in 120 mL ethanol and the insoluble sodium nitrate was filtered. Crude NOArg-4 monomer was collected from the ethanol solution using a rotary evaporator (Buchi, R110), then purified by recrystallization three times in cold isopropyl alcohol (precipitate at −20° C.) and vacuum-dried at room temperature overnight. Arg-4-CL monomer was synthesized using the same method in the prior studies of the Arg based PEAs and PEUUs. The detailed procedures were described elsewhere.

Synthesis of the NOArg PEA Homopolymer and NOArg-Arg PEAs Copolymers.

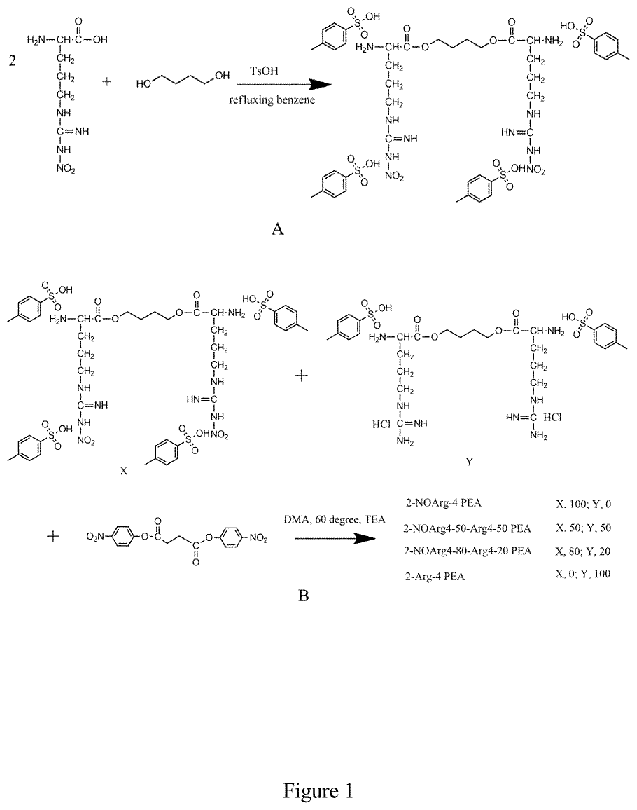

Both homopolymers (2-NOArg-4 PEA, 2-Arg-4 PEA) and copolymers at different composition ratios of NOS inhibitor to Arg building blocks (2-NOArg-4-Arg-4 50/50 PEA, 2-NOArg-4-Arg-4 20/80 PEA) were synthesized by a solution polycondensation of Arg-4-CL, NOArg-4 and p-nitrophenol diester monomers via prior published polycondensation procedures. Briefly, the polymerization was carried out in DMAc solution in an oil bath at 70° C. for 30 h and triethylamine was used as an acid receptor for TsOH. The stoichiometric ratio of amino acid diester (NOArg-4 and Arg-4-CL monomers in total) to p-nitrophenol diester is 1:1. The resulting NOArg-Arg coPEAs are labeled as y-NOArg-x1-Arg-x2, where y, x1 and x2 are the numbers of CH₂ groups in diacid and diol segments, respectively (FIG. 1 and Table 1). The 2-NOArg-4 PEA is a 100% NOS inhibitor-based PEA, while the 2-Arg-4 is a 100% Arg-based PEA. The rest samples are the Arg-based copolymers having both Arg and NOS inhibitor building blocks. All Arg-based PEA samples were carefully purified to prevent the cytotoxicity from chemical residuals and stimulants to the immune cells. After the polymerization, the Arg-based PEA solutions in DMAc were poured and precipitated in acetone to remove the most of the p-nitrophenol, triethylamine, etc. The precipitated Arg-based PEA was filtered, vacuum-dried, dissolved in Milli-Q water and further purified by dialysis against Milli-Q water in autoclaved glass beakers (3 L, 6 h, MWCO: 3,500 g/mol) until no yellow color of p-nitrophenol could be detected (water was changed 6-8 times). The purified sample solution in dialysis tubes was transferred to sterilized centrifuge tubes, lyophilized and stored at 4° C.

Characterizations of NOArg-4 and NOArg-Arg PEAs.

The NOArg-4 and NOArg-Arg PEA series were analyzed by proton nuclear magnetic resonance (¹H-NMR). ¹H-NMR spectra were recorded on a Varian (Palo Alto, Calif.) INOVA-400 spectrometer at 400 MHz. Deuterated dimethyl sulfoxide (DMSO-d6, Cambridge Isotope Laboratories) was used as the solvent. The sample concentration in DMSO-d₆ was about 1% (w/v). All of the chemical shifts were reported in parts per million (ppm). Fourier transform infrared (FTIR) spectra of the NOArg-4 and NOArg-Arg PEA series were recorded on a PerkinElmer (Madison, Wis.) Nicolet Magna 560 FTIR spectrometer with Omnic software for data acquisition and analysis. All solubility tests were performed in glass vials at room temperature. Gel permeation chromatography (GPC) of NOArg-Arg PEA series was done on a Waters aqueous GPC equipped with a Waters 410 differential refractive index detector. The injection volume of the polymer sample solution (5 mg/mL in Milli-Q water) was 100 μL. Polyethylene oxide standards were used for calibration.

In Vitro Degradation of 2-NOArg-4 PEA and NOArg-Arg PEAs by Sakaguchi Test.

The degradation of 2-Arg-4 PEA and NOArg-Arg PEA series was evaluated by Sakaguchi test quantitatively. 20 mg of each type of Arg-based PEA samples (2-NOArg-4, 2-NOArg-4-Arg-4 20/80, 2-NOArg-4-Arg-4 50/50) was dissolved in 5 mL DI water at room temperature then loaded in dialysis tubes (10 mm diameter, MWCO: 1,000 g/mol). Dialysis tubes were immersed in 95 mL 0.1 M pH 7.4 PBS and incubated at 20° C. and 37° C. At predetermined intervals, 1 mL immersion PBS was sampled three times at predetermined intervals and 3 mL fresh PBS was added to compensate the immersion solution volume. The 1 mL immersion PBS sample solution removed was diluted with DI water to 5 mL in test tubes and cooled on ice. 1 mL 10% NaOH solution and 1 mL 0.02% α-naphthol ethanol solution were added and mixed in test tubes embedded in ice. After 1 h, 200 μL hypobromite solution was added with shaking, then 1 mL 40% urea solution was added to each test tube. The absorbance of the solutions at 515 nm was tested using a UV-Vis spectrophotometry (PerkinElmer Lambada 35, Madison, Wis.). A calibration curve of Arg concentration can be established by assaying 5 mL Arg solution in DI water with a linear range from 2-30 μg/mL. Sakaguchi test is usually considered specific to the presence of Arg in solutions or in proteins. However, the water-soluble derivative of NOArg, such as L-NAME, has an absorbance at 515 nm but is lower (about 26% absorbance at equal molar concentration) than Arg in the Sakaguchi test. Because the water-soluble derivative of NOArg has a different absorbance at 515 nm compared to Arg in the Sakaguchi test, the data of degraded NOArg PEA and NOArg-Arg PEAs in PBS were normalized against that of completely hydrolyzed samples. Briefly, 2 mg of NOArg-Arg PEA samples were hydrolyzed in 1 mL 0.2 M NaOH solution at 40° C. for 2 h, vacuum dried, dissolved in 20 mL DI water, diluted to 5 mL with DI water and assayed. The average of 3 assays of each type of the Arg-based PEA sample was considered as the absorbance at 100% degradation.

Cytotoxicity of NOArg-4 and NOArg-Arg PEAs.

3T3 fibroblasts were maintained in Dulbecco's modified eagle medium (DMEM) supplemented with 10% fetal bovine serum (FBS, Mediatech, VA) and 100 U/mL penicillin-streptomycin. RAW 264.7 cells were maintained in DMEM supplemented with 10% delipidized FBS (endotoxin <0.3 EU/mL, Gemini Bio-products, CA), 1% 1 M 4-(2-hydroxyethyl)-1-piperazineethanesulfonic acid (HEPES) buffer, 1% 100 mM sodium pyruvate, 1% 200 mM L-Glutamine and 100 U/mL penicillin-streptomycin.

The cytotoxicity of NOArg-4 and NOArg-Arg PEA series against RAW 264.7 cells and 3T3 fibroblasts was evaluated by a standard MTT assay. Cells were seeded in a 96-well plate at a density of 8×10³ cells/well. All NOArg-4 and NOArg-Arg PEA samples were dissolved in complete DMEM media and sterilized by filtering through 0.22 μm filters (Pall Corporation, NY). 1 mg/mL or 2 mg/mL NOArg-4 and NOArg-Arg PEA series in DMEM supplemented with 10% FBS were incubated along with RAW 264.7 or 3T3 cells for 24 and 48 h. Cells cultured in media were used as controls. After incubation, the cell media was removed, washed with PBS, and 100 μL of media containing 10 μL of 5 mg/mL MTT solution (thiazolyl blue tetrazolium bromide in PBS filtered by a 0.22 μm filter) was added to each well following another 4 h of incubation. 150 μL of DMSO was added to each well after removing the MTT media, and the 96-well plate was gently shaken for 30 min at room temperature. The absorbance of the DMSO solution was measured at wavelengths of 570 and 690 nm (Spectramax plus 384, Molecular Devices, U.S.A.). The cell viability (%) was calculated according to the following equation:

$$Viability ( \% ) = \frac{OD 570 ( sample ) - OD 690 ( sample )}{OD 570 ( control ) - OD 690 ( control )} \times 100 \%$$

NO production and arginase activity assay of RAW 264.7 cells treated with NOArg-4 and NOArg-Arg PEAs

4×10⁵ RAW 264.7 cells in each well were incubated in 400 μL DMEM media contains 10% FBS in 24 well plates for 24 h. For the resting macrophages group, media was then removed and replaced with treatments of 1 mg/mL or 2 mg/mL Arg, L-NAME, Arg+L-NAME (1:1 weight ratio mixed), NOArg-4 monomer, 2-NOArg-4 PEA, 2-NOArg-4-Arg-4 50/50 PEA, 2-NOArg-4-Arg-4 20/80 PEA or 2-Arg-4 PEA in DMEM supplemented with 5% FBS. For CAM or AAM group, treatments contained either 100 U/mL recombinant mouse interferon-γ (IFN-γ, 1.25×10⁷ U/mg, Shenandoah Biotechnology, PA) plus 100 ng/mL lipopolysaccharide (LPS, Sigma-Aldrich) or 20 ng/mL recombinant mouse interleukin-4 (IL-4, Shenandoah Biotechnology, PA) respectively to induce different activation phenotype. Resting Macrophages, CAM and AAM incubated in DMEM contains 5% FBS without treatments were used as controls.

After 24 h treatment, the NO production in the supernatant media was measured by Griess Reagent according to the manufacture's protocol (Enzo life Science, NY). The absorbance at 540 nm was measured within 30 min in a plate reader (Spectramax plus 384, Molecular Devices, U.S.A.). The arginase activity assay is tested colorimetrically. The cells were washed with PBS and lysed by 200 μL 0.2% Triton X-100 in 20 mM Tris-HCl, pH 7.5 containing 20 μg pepstatin A, 20 μg aprotinin and 20 μg leupeptin as protease inhibitors. After the cell was lysed, 200 μL 10 mM MnCl₂ was added and the enzyme was activated for 10 minutes at 55° C. 100 μL aliquot of the activated lysate was mixed with 100 μL 0.5 M Arg and incubated at 37° C. for 1 h. The reaction was stopped by the addition of 800 μL mixed acid solution (H₂SO₄/H₃PO₄/H₂O=1/3/7) and 100 μL 9 wt % α-isonitrosopropiophenone in ethanol solution. The mixture solution was heated in a 95° C. water bath for 45 min. After being cooled to room temperature, the absorbance of 200 μL mixture solution at 540 nm was tested in 96 well plates. A urea calibration curve was prepared with increasing amounts of urea between 1.5 and 30 μg. One unit (U) of arginase activity was defined as the enzyme activity that catalyzes the production of 1 μM of urea per minute under the condition of the assay. The NO production and the arginase activity in three replicate wells of each condition were tested and the mean value was calculated with standard deviations.

TNF-α and TGF-β1 production of RAW 264.7 cells treated with NOArg-Arg PEA series.

The cell culture and treatment of 4×10⁵ RAW 264.7 cells were the same as described in the above section. TNF-α and TGF-β1 production were measured by ELISA according to the manufacturer's instructions (mouse TNF-α standard TMB Elisa kit, Peprotech, NJ and TMB substrate set, Biolegend, CA were used for the TNF-α assay and TGF beta 1 ELISA kit, affymetrix, CA was used for the TGF-β1 assay). Three replicate wells were assayed, and the mean value was calculated with a standard deviation.

In vivo wound healing study in diabetic rat model.

Induction of the type I diabetic rat model and surgical procedures.

250 to 300 g weighed Sprague-Dawley (SD) rats were obtained from Laboratory Animal Center, Guangdong Pharmaceutical University. All rats were housed and fed in a specific pathogen-free (SPF) environment during the course of the study. All animal procedures were approved by the institutional animal care and use committee. Diabetes was induced by intraperitoneally injecting 1% streptozotocin (STZ, 65 mg/kg) dissolved in 0.1 M sodium citrate buffer. After 7 days, rats were considered diabetic rat models, if the blood glucose level was higher than 16.7 mM, together with the observations of weight loss, polyuria and polydipsia.

Pluronic F-127 gels containing 10 mg/mL NOArg-PEA and Arg-PEA homopolymers, and NOArg and Arg-based PEA copolymers were used as the treatment formulations. Each of these biomaterials were mixed with 30% w/v Pluronic F-127 solution. The diabetic rats were anesthetized using 10% chloral hydrate (300 mg/kg body weight). After the dorsal hair of rats was shaved and sterilized, two circular, full-thickness skin wounds with the diameter of 18 mm were created on the back of each rat. Rats were then randomized into four groups (n=4) and treated with Arg PEA, NOArg PEA, 20/80 PEA, 50/50 PEA gels or Pluronic F-127 gel (control). Finally, all wounds and surrounding areas were covered with 3M Tegaderm™ (3M Health Care, USA). The treated rats were fed in individual cages.

Wound Healing Assessment.

The healing status in each group was evaluated by images of the wound area on days 0, 3, 7, and 14 post-surgery. Wound areas were calculated using image analysis software Image-Pro Plus (Media Cybernetics, USA). The degree of healing was expressed as the wound healing rate: (A₀−Aₜ)/A₀×100%, where A₀ is the initial wound area (t=0) and Aₜ is the wound area at time t.

Histological and Immunohistochemical Analysis.

The quality of wound area during healing was assessed by both histological and immunohistochemical data. Rats were sacrificed at 3, 7 and 14 days post-surgery, and the wounded tissue with its surrounding rim of intact skin together were harvested and fixed in 4% paraformaldehyde followed by standard dehydration with gradient ethanol and embedded in paraffin wax. Subsequently, skin tissues were cut into 5 μm sections for both histological and immunohistochemical evaluations. For histological analysis, hematoxylin and eosin (H&E) staining for cellular types and contents and Masson's Trichrome staining for collagen contents were performed. For the immunohistochemical staining, sections were deparaffinized, redehydrared and incubated with primary antibodies against arginase I (Arg I sc-18354, Santa Cruz sc-18354, USA, 1:100), nitric oxide synthase-2 (NOS2, Santa Cruz sc-651, USA, 1:100), CD80 (Bioss 2211R, China, 1:500) and CD206 as biomarker for M2 macrophage (Bioss 4727R, China, 1:500) overnight at 4° C. Then they were incubated with streptavidin-HRP secondary antibody (DAKO, USA) for 1 hour. Immunodetection was visualized by a diaminobenzidine (DAB) detection kit (Servicebio, China). The stained slides were imaged by a light microscope (Axioskop 40 FL, Zeiss, Germany).

Results and discussion. Synthesis of NOArg-4 monomers, NOArg-PEA homopolymers and NOArg-Arg PEA copolymers.

The NOArg-4 monomers were synthesized from NOArg and 1,4-butanediol in refluxing benzene (FIG. 1 A). NOArg-x can be considered as a N^{ω}-nitro protected Arg derivative synthesized by nitrification reaction in sulfuric acid and the removal of the nitro group (deprotection) usually requires catalysts and strong acids. However, when heating in refluxing toluene (b.p. 110° C.) which is used for most esterification of amino acids could lead to a partial removal of the nitro group (tested by FTIR and Sakaguchi test). The esterification of NOArg and n-butanediol in refluxing benzene (b.p. 80° C.) prevented the removal of nitro group. Also, the reactants were mixed with sodium nitrate (insoluble in benzene) to prevent locally overheating when the viscosity of the reaction increased in the esterification process. The procedures for the synthesis of NOArg-PEA homopolymers and NOArg-Arg PEA copolymers were similar to the solution polycondensation scheme. Table 1 lists the synthesized NOArg-PEA homopolymers and NOArg-Arg PEA copolymers and their monomer feed ratios, sample labels and molecular weights.

TABLE 1
Molecular weight of NOArg-Arg copolymers.

| | Monomers molar | | | | |
|---|---|---|---|---|---|
| | feed ratio | | | | |
| | (NOArg-4:Arg-4:di- | | | | |
| | nitrophenol | Sample | Mₙ | M_{w} | |
| | succinate) | Label | (g/mol) | (g/mol) | PDI |
| 2-NOArg-4 | 10:0:10 | NOArg-PEA | 18,800 | 26,700 | 1.42 |
| (100% NOArg | | | | | |
| homopolymer) | | | | | |
| 2-NOArg-4-Arg-4 | 2:8:10 | 20/80-PEA | 21,600 | 31,400 | 1.45 |
| 20/80 | | | | | |
| 2-NOArg-4-Arg-4 | 5:5:10 | 50/50-PEA | 19,700 | 29,100 | 1.48 |
| 50/50 | | | | | |
| 2-Arg-4 | 0:10:10 | Arg-PEA | 19,800 | 27,100 | 1.37 |
| (100% Arg-PEA | | | | | |
| homopolymer) | | | | | |

Chemical and Material Characterizations.

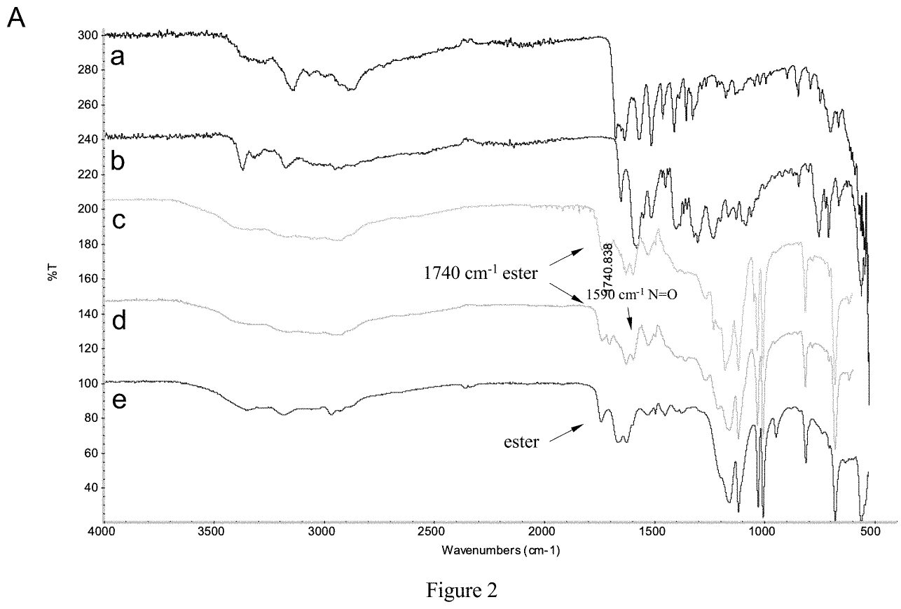

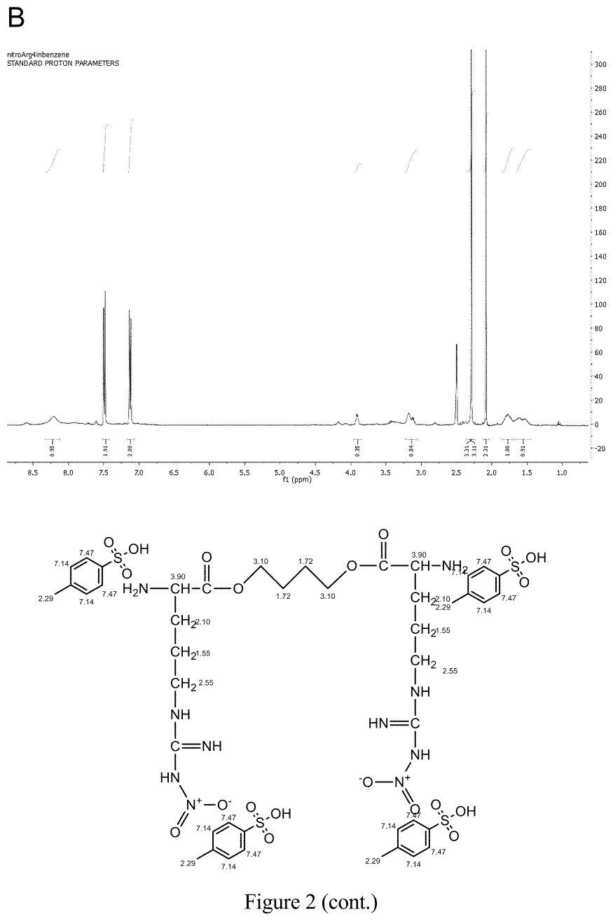

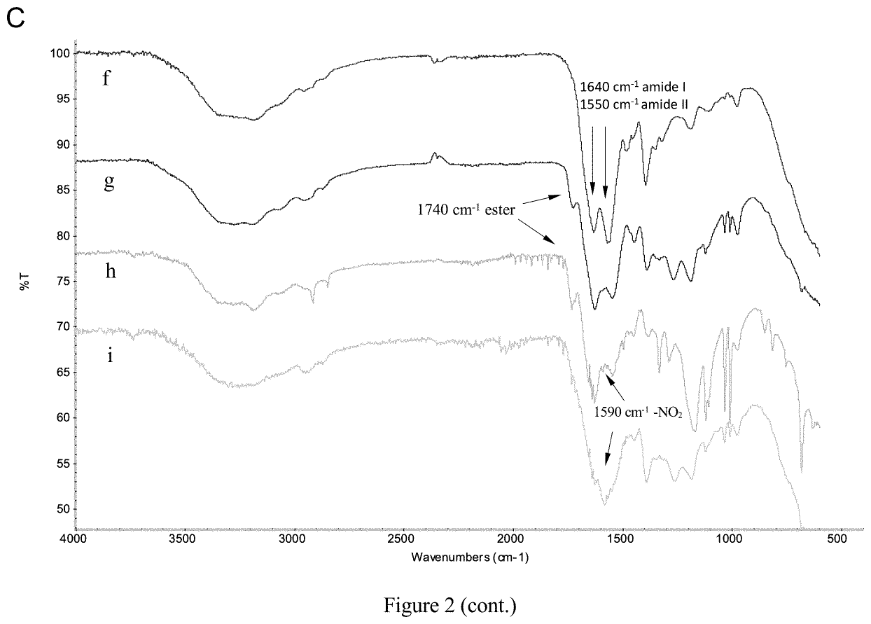

NOArg-4 monomer and N-nitro-L-arginine methyl ester (L-NAME) showed ester absorbance at 1,740 cm⁻¹ and the asymmetric vibration of NO₂ (vₐₛ (NO₂)) absorbance at 1590 cm⁻¹ (FIG. 2A). NOArg-4 monomer synthesized in toluene has less vₐₛ (NO₂) absorbance than NOArg-4 synthesized in benzene, suggesting the nitro group on the guanidine can be partially removed in refluxing toluene. The chemical structure of NOArg-4 was confirmed by H-NMR (FIG. 2B). Tetra-p-toluene sulfonic acid salt of bis (L-NOArg) butane diester (NOArg-4): H-NMR (DMSO-d6, ppm, δ): 1.55 [4H, —OC(O)—CH(NH₃⁺)—CH₂—CH₂—CH₂—NH—], 1.72 [4H, —(O)CO—O—CH₂—CH₂—], 2.10 [4H, —OC(O)—CH(NH₃⁺)—CH₂—CH₂—CH₂—NH—], 2.29 [12H, H₃C-Ph-SO₃—], 2.55 [—CH₂—CH₂—CH₂—NH—], 3.10 [4H, —(O)C—O—CH₂—CH₂—], 3.90 [2H, H₂N—CH(R)—C(O)—O—], 8.22 [4H, H₂N—CH(R)—C—(O)—O—]. The synthesis and chemical characterization of Arg-4-Cl monomer and the di-nitrophenyl succinate monomer used in the copolymerization had been described in prior studies. FTIR spectra of synthesized Arg-PEAs clearly shows the absorbance of the amide I and amide II bond formed in the polymerization at 1,640 cm⁻¹ and 1,550 cm⁻¹ (FIG. 2C). As the NOArg content increases in the copolymer, the vₐₛ (NO₂) absorbance of 2-NOArg-4-Arg-4 50/50 PEA and NOArg-4 PEA also presents at 1,590 cm⁻¹ on the spectra.

Similar to L-NAME, NOArg-4 monomer, 2-NOArg-4 PEA and NOArg-Arg co-PEAs are water soluble due to the carboxyl group of NOArg forms ester bond with the hydroxyl groups of butane diol. As the composition of NOArg block in the copolymer increases, the solubility in water becomes lower. 2-NOArg-4 PEA, 2-Arg-4 PEA, and NOArg-Arg copolymers could also be dissolved in DMAc, DMF, and DMSO (>5 mg/mL) at room temperature, but are insoluble in acetone, chloroform, dichloromethane, tetrahydrofuran, methanol, ethanol etc. The number average molecular weight (Mₙ) of 2-NOArg-4 and NOArg-Arg co-PEA tested by aqueous GPC ranged from 18,800 to 21,600 g/mol, similar to the Mₙ of the prior synthesized 2-Arg-4 PEA homopolymer.

2-NOArg-4 PEA and NOArg-Arg Co-PEA Degradation In Vitro.

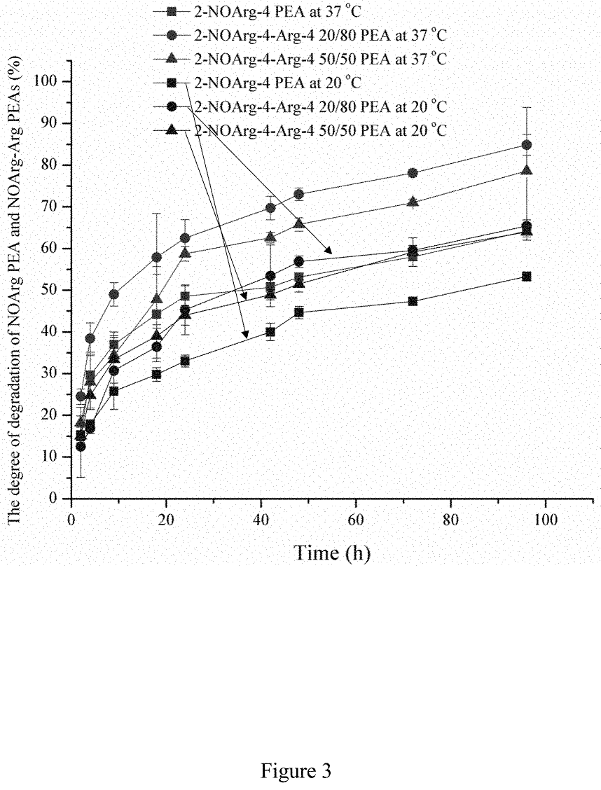

The level of hydrolytic degradation of 2-Arg-4 PEA and NOArg-Arg co-PEA at different polymer composition levels and temperature is shown in FIG. 3. In general, higher NOArg composition/lower Arg composition in co-PEA achieved slower degradation rate and materials degraded faster at higher temperature, i.e. degradation at 37° C. is faster than that at 20° C. NOArg PEA, 50/50 PEA, 20/80 PEA achieved 48.5%, 58.8% and 62.5% degradation at 24 h, and 64.2%, 78.6% and 84.9% degradation at 96 h, respectively (day 4) at 37° C. Due to the relatively higher hydrophobicity of the NOArg PEA, the NOArg-Arg co-PEAs having higher NOArg-4 (x block) contents (e.g., 50/50 PEA) showed a slower degradation rate than the copolymer having lower NOArg-4 contents (e.g., 20/80 PEA).

The degradation data are useful because the data provide the timeline of the amounts of NOArg and Arg released from the biomaterials in a simulated physiological environment. The inhibitory effect of NOArg PEA and NOArg-Arg PEAs on iNOS activity depends on the available concentration of those small molecule NOArg derivatives and free Arg released in the AA-PEA degradation process. In this disclosure, the biodegradable NOArg-Arg copolymers supplied free Arg substrate and suppressed the over-production of NO at the wound injury site at the same time. 50/50 PEA and 20/80 PEA achieved higher than 70% degradation in vitro at 37° C. on day 4. The degradation rate of these Arg-based PEAs in vivo is probably faster than in vitro because of the presence of proteases within the wound tissue. The in vitro degradation data can also provide the scientific base for the design of the timeline of our subsequent in vivo trials.

The supplement of Arg and NOS inhibitors used in wound in situ is expected to influence the healing process. Most Arg catabolism through iNOS is conducted by macrophages that present in a wound site about 3 days after injury and dominate about 7-12 days in a normal healing process. Arg is the only amino acid whose concentration in a wound fluid decreased over time to undetectable levels. Protein breakdown does not contribute to Arg and NO production. Dietary Arg supplementation has shown beneficial effects on acute incisional wounds. However, no evidence shows dietary supplemental Arg improved chronic wounds (e.g., pressure ulcer, diabetic ulcer). Local early Arg supplementation (infusion or topical application) may disturb the reciprocal regulation of iNOS and arginase, leading to the preferential metabolism of Arg to excess NO production with a consequent reduction in angiogenesis and granulation tissue formation. An excessive amount of NO could be inhibitory to the wound healing, particularly in the granulation tissue formation stage. High levels of NO produced by iNOS interacts with oxidative molecules (superoxide is the main component) derived from neutrophils and macrophages (via the phagocytic isoforms of NADPH oxidase) to produce peroxynitrite which induces apoptosis/necrosis of fibroblasts and many other types of cells in the healing process. When Arg and L-NAME were topically applied together, the hydroxyproline synthesis of the wound tissue was elevated.

Cytotoxicity Test of NOArg PEA and NOArg-Arg PEA.

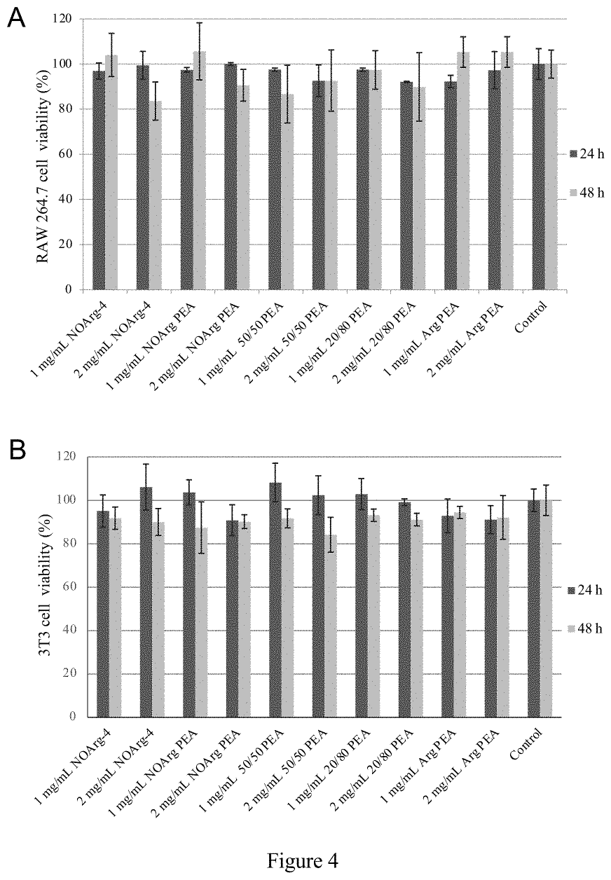

As shown in FIG. 4, irrespective of monomer vs. polymers, copolymer composition, and cell types, all testing samples (NOArg-4 monomer, NOArg PEA and NOArg-Arg co-PEA) showed no statistically significant (p<0.05) cytotoxicity toward RAW 264.7 macrophages and 3T3 fibroblasts from the control up to 2 mg/mL concentration in 24 h and 48 h. For example, at 48 h, the viability of macrophages treated with 2 mg/mL NOArg PEA, 50/50 PEA, 20/80 PEA, Arg PEA ranges from 90.6%±7.0%, 92.7%±13.5%, 89.9%±15.2%, to 105.3±6.8%, respectively, and the viability of 3T3 fibroblasts with the same treatment are 90.2%: 3.2%, 84.1%±8.0%, 91.1%±2.9%, 92.1%±10.1%, respectively.

Macrophages are prominent inflammatory cells that have many functions in wound healing. Fibroblasts and myofibroblasts produce provisional matrix and a local contraction of the matrix within the granulation tissue of healing wounds to reconstitute the tissue continuity. The NOArg PEA and NOArg-Arg co-PEAs showed good biocompatibility to both these two important cell types in the inflammatory and granulation formation stages of wound healing. The cell biocompatibility data in this disclosure are consistent with other published biocompatibility data of other types of water soluble and insoluble Arg-based PEA and its derivatives and water insoluble Arg-based PEUU. Based on the FIG. 4 cell biocompatibility data, 1 mg/mL and 2 mg/mL of AA-PEA samples in media were used to treat macrophages in the following NO and cytokine production in vitro studies, etc.

NO Production and Arginase Activity of RAW 264.7 Cells Treated with NOArg-Arg PEAs.

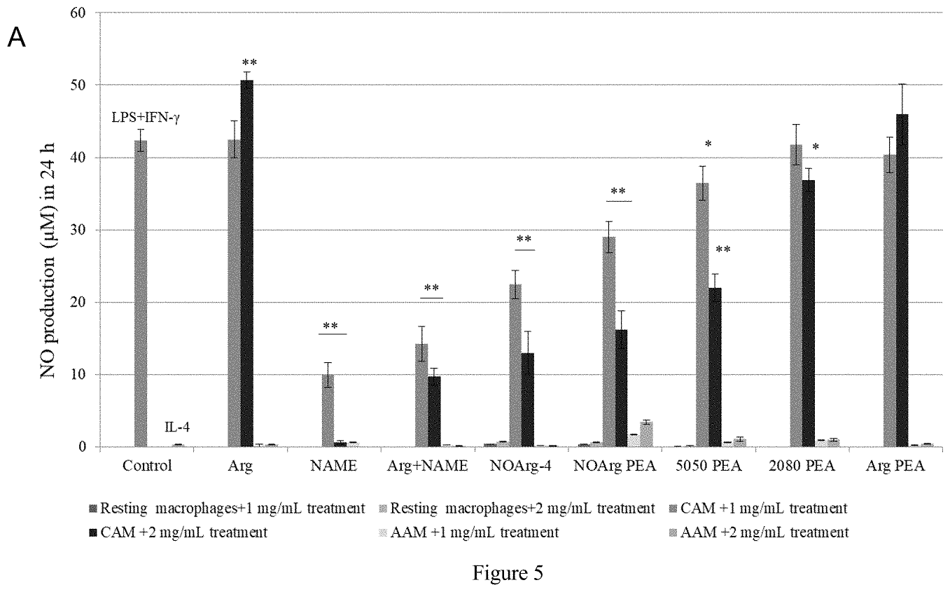

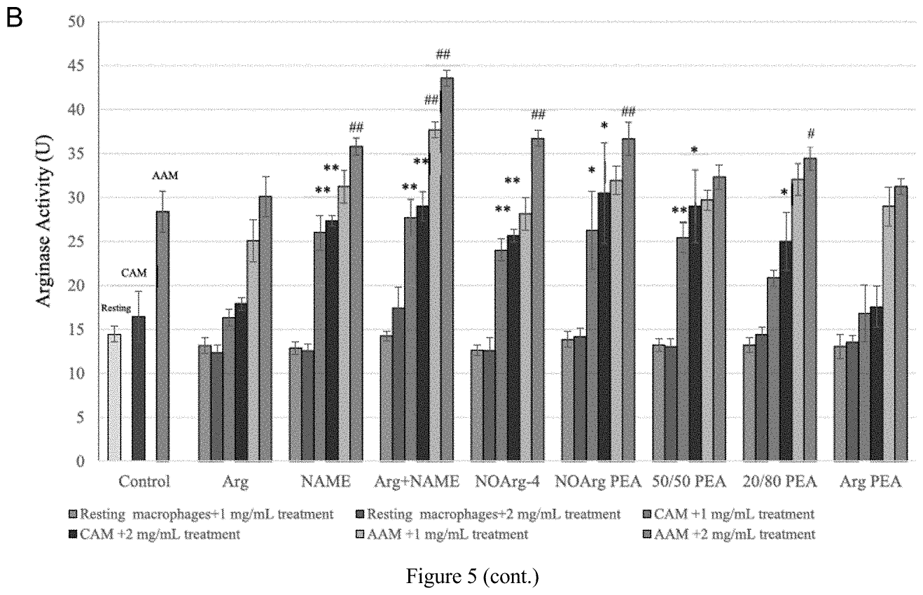

As shown in FIG. 5A, classically activated macrophages (CAM) produced a much higher NO level when compared to the resting macrophages or the alternative activated macrophages (AAM). For example, the CAM show NO level from low 10 to high above 50 μM, while the resting macrophages and AAM (alternative activated macrophages) treated with NOArg PEA or NOArg-Arg PEA produced a very low-level NO from undetectable to 3.4 μM. Increasing extracellular Arg concentration elevated the NO production of CAM, i.e. NO 50.7±1.1 μM at 2 mg/mL Arg>NO 42.5±2.6 μM at 1 mg/mL Arg≈≈42.4±1.5 μM at CAM control; this is so-called Arg paradox: the dependence of cellular NO production on exogenous Arg concentration despite the theoretical saturation of NOS with intracellular Arg.

Among all the Arg-based testing samples, L-NAME suppressed the NO production the most in the CAM case, 9.9±1.7 μM NO at 1 mg/mL and 0.6±0.2 μM NO at 2 mg/mL. L-NAME is a low toxic, stable iNOS inhibitor that is not degradable by arginase. When treating cells and tissues by L-NAME, the effective concentration of L-NAME ranged from 0.1 (27 μg/mL) to 10 mmol/L (2.7 mg/mL). NOArg-4 monomer is a less potent inhibitor when compared to L-NAME (22.4±2.0 μM NO from NOArg-4 monomer vs. 9.9±1.7 from L-NAME at 1 mg/mL, and 13.0±2.9 μM NO from NOArg-4 monomer vs. 0.6±0.2 from L-NAME at 2 mg/mL), probably because L-NAME can efficiently compete with Arg to bind to the oxygenase domains of iNOS, whereas NOArg-4 is a larger molecule with two nitroguanidine groups which are not always bound to two iNOS simultaneously.

Both 2-NOArg-4 PEA and NOArg-Arg co-PEA contained NOArg-4 building blocks, but showed a different level of inhibitory effect on NO production. NOArg-4 PEA having a higher level of NO inhibition. It is attributed to the different level of the NOArg contents in these two different types of AA-PEAs designed. The inhibitory effect of NOArg-Arg co-PEAs is less potent than a pure 100% NOArg PEA homopolymer (29.0±2.1 μM NO at 1 mg/mL, 16.2±2.6 μM NO at 2 mg/mL), i.e. 50/50 PEA (36.5±2.4 μM NO at 1 mg/mL, 22.0±1.9 μM NO at 2 mg/mL), and 20/80 PEA (41.8±2.8 μM NO at 1 mg/mL, 36.9±1.6 M NO at 2 mg/mL). The NO level of a pure Arg PEA (no NO inhibiting component) treated CAM (40.4±2.4 μM NO at 1 mg/mL, 46.0±4.2 μM NO at 2 mg/mL) showed no statistically significant difference from the CAM control (treated by LPS+IFN-γ). By adjusting different dosage and composition of NOArg-Arg co-PEA in the treatments, the concentration of NO produced by CAM could be finely controlled. The tunable inhibitory effect of NOArg-Arg co-PEAs may provide an alternative approach to adjust the NO production level of the local wound bed to maximize its beneficial effect toward wound healing.

In addition to the NO production upon activated macrophages, they can also be directed toward arginase production to facilitate wound healing. As shown in FIG. 5B, the CAM incubated with the biomaterials' treatments, however, showed statistically significantly higher arginase activity than the CAM control (16.5±2.9 U), for examples, at 2 mg/mL treatment, the arginase activity level ranges from the highest 30.5±7.7 U from 2-NOArg-4 PEA followed by 29.0±4.1 U from 2-NOArg-4-Arg-4 50/50, 29.0±1.6 U from Arg+L-NAME, 27.4±0.6 U from L-NAME, 25.0±3.3 U from 2-NOArg-4-Arg-4 20/80 to the lowest 25.7±0.7 U from NOArg-4 monomer. Similar arginase data trend was observed at the 1 mg/mL concentration treatment. These arginase data suggest that a higher NOArg composition in the NOArg-Arg co-PEA biomaterial treatment showed a higher arginase activity in CAM, i.e. 2-NOArg-4 PEA>2-NOArg-4-Arg-4 50/50>2-NOArg-4-Arg-4 20/80. Both the Arg (17.9±0.7 U) and 2-Arg-4 PEA (17.6±2.3 U) treatments up to 2 mg/mL have no statistical significant different arginase level when compared to the CAM control. In the resting macrophage condition, extracellular Arg concentration, iNOS inhibitors (L-NAME) treatment and Arg-based PEA treatments have no statistically significant effect on the arginase expression (12.3 U-17.4 U) from the control (14.5±0.9 U) as shown in FIG. 5B.

There are two isoforms of arginase, arginase I (cytosolic) and arginase II (mitochondrial), and arginase can hydrolyze Arg to produce urea and L-ornithine in macrophages. AAM dominantly express arginase I, while arginase II is associated with pro-inflammatory responses. CAM upregulates iNOS and arginase II, but not arginase I in murine and human macrophages. In the arginase data shown in FIG. 5B, it can be concluded that the Arg-based copolymers having a higher content of NOS inhibitor component like NOArg have the potential to direct macrophage response more toward M2 phenotype via arginase expression than M1 type which is more NO and less arginase production. The arginase data in FIG. 5B also show that all the 2-NOArg-4 PEA and its Arg co-PEAs (2-NOArg-4-Arg-4) had the similar as or higher level of arginase expression than the AAM control.

The possible explanation to the arginase activity change after the variety of treatments is because N-o-hydroxy-L-arginine, which is an intermediate in the NOS pathway and the nitrite which is the oxidized product of NO, can inhibit arginase activity. In the current in vitro test, the nitrite and N-o-hydroxy-L-arginine produced by CAM control and Arg PEA treated CAM without iNOS inhibition showed lower the arginase activity compared to 2-NOArg-4 PEA and NOArg-Arg PEAs in FIG. 5B.

In the AAM case, all treatments showed the highest level of arginase activity (mainly arginase I) than the corresponding resting macrophages and CAM control, but only 3 types of treatments showed a statistically higher level of arginase activity than the AAM control (28.4±2.3 U), and those 3 types of treatments with 2 mg/mL are: NOArg-4 (36.7±1.9 U), NOArg-4 PEA (36.7±1.9 U) and 2-NOArg-4-Arg-4 20/80 PEA (34.4±1.3 U). Arginase I is anti-inflammatory which is dominantly expressed in the AAM and reduces NO production from iNOS through limiting intracellular Arg. Small molecular weight NOS inhibitors led to a significantly higher arginase activity in AAM, i.e., L-NAME (31.2±1.9 U, 35.8±1.0 U), 2 mg/mL NOArg-4 (36.7±1.9 U). The possible explanation is that the arginase I gene expression is inducible in macrophages by a variety of stimuli besides IL-4, for example, by elevated cAMP, GM-CSF, IL-10 and TGF-β. Small molecule NOS inhibitor treatment might influence the level of these factors which can also upregulate the expression of arginase I.

After the acute inflammatory stage of wound healing, NO production is suppressed, resulting in an increase in arginase activity from macrophages. In acute wound healing stage, arginase is synthesized mainly by macrophages and released into the wound environment through the apoptosis of macrophages at the end of inflammatory stage. The arginase activity from macrophage may also participate in the fibrogenic process via the synthesis of ornithine-derived proline and anti-inflammatory action via production of polyamines. An elevated arginase activity of macrophages in the late inflammatory stage may also decrease the NO level which is inhibitory to fibroblast proliferation. Notably, the arginase activity of fibroblasts could also be induced by IL-4 and TGF-β1.

TNF-α and TGF-β1 Production of RAW 264.7 Cells Treated with NOArg-Arg PEAs.

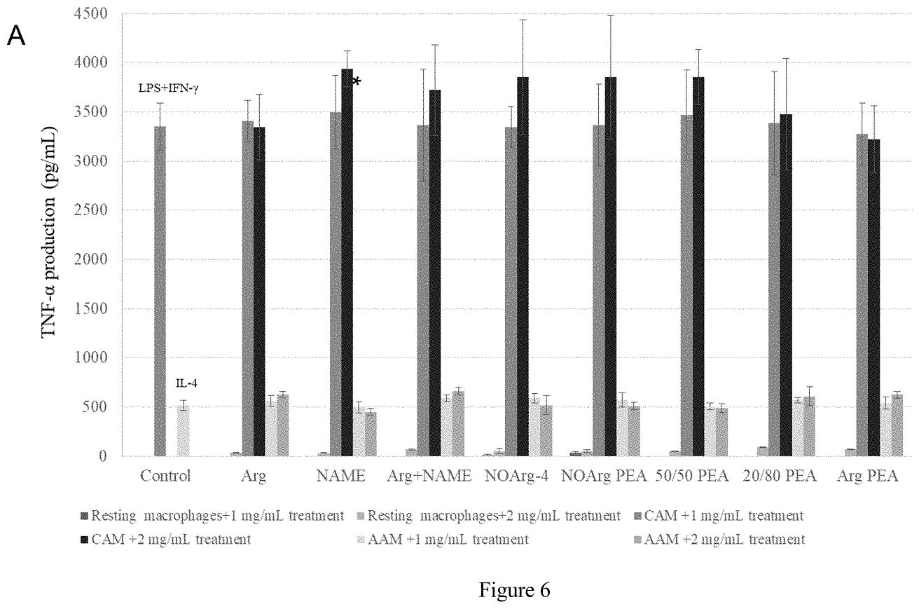

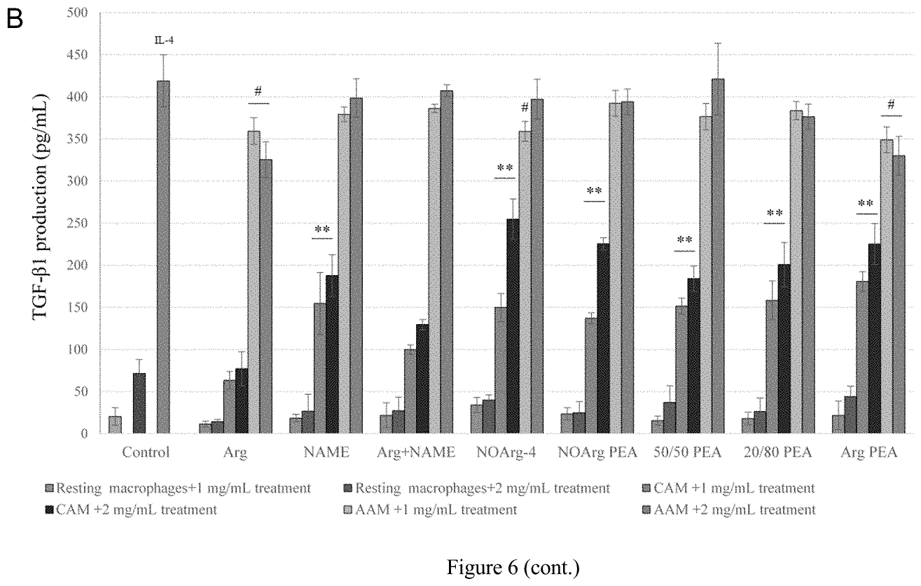

TNF-α produced from the 3 phenotypes of macrophages (resting, CAM and AAM) after treatment with the Arg-based PEAs is shown in FIG. 6A to evaluate the effect of different treatments on the level of proinflammatory mediators. No TNF-α production was detected from the resting macrophages control. After treatment by NOArg-4 PEA, 2-NOArg-4-Arg-4 50/50 PEA or 2-NOArg-4-Arg-4 20/80 PEA, resting macrophages produced a low level of TNF-α (0-88.2 μg/mL), but statistically insignificant from the control. The CAM control condition produced higher than 6 folds of TNF-α (3,349.4±243.3 μg/mL) when compared to the AAM control condition (518.3±52.5 μg/mL). For example, in the CAM condition, the 2 mg/mL treatments of Arg+L-NAME, NOArg-4 monomer, NOArg PEA, 50/50 PEA, and 20/80 PEA resulted in TNF-α production of 3,722.2±461.6, 3,855.8±579.9, 3,851.7±625.9, 3,856.1±277.1, and 3,474.5±569.1 μg/mL, respectively. Treatments under the CAM condition produced slightly higher TNF-α than the CAM control (LPS+IFN-γ), but only the 2 mg/mL L-NAME treatment (3,937.0±183.9 μg/mL) produced a statistically significantly higher TNF-α than the CAM control. No significant change of TNF-α level from all the treatments in the AAM condition was found when compared with the AAM control.

CAM is characterized by the production of proinflammatory cytokines like TNF-α, IL-1, and IL-6 in the early stage of inflammatory response in wound repair, whereas AAM produces fewer TNF-α and elevated arginase I in the later stage of wound repair. TNF-α can indirectly promote reepithelialization and synergizes with IFN-γ inducing NO production. A low level of TNF-α can promote wound healing by indirectly stimulating inflammation and increasing macrophages produced growth factors. But, at a high level and prolonged time, TNF-α has a detrimental effect on healing that is usually observed in chronic wounds, such as diabetic wounds. Although iNOS and TNF-α are two independent pathways, some studies reported NOS inhibitors could cause a concentration dependent increase of TNF-α release from CAM in vitro and in vivo. One possible reason is NO inhibited NF-κB activity, hence downregulate COX-2 gene expression resulting in an increase production of PGE₂. PGE₂ inhibits TNF-α release through elevation of cAMP. Another possible pathway is NO inhibits the activation of transcription factor NF-κB which is required in the TNF-α mRNA formation. As shown in FIG. 6, NOArg PEA and NOArg-Arg co-PEAs inhibit the NO production, but they do not inhibit the pro-inflammatory cytokine production. Our data show the production of TNF-α from CAM were not significantly influenced or slightly increased by the iNOS pathway inhibition probably our NOArg-PEA and NOArg-Arg PEA treatments are not inhibitory enough to show the significantly increased TNF-α level. And iNOS inhibition can also increase the level of IL-6 from CAM was reported.

FIG. 6B shows resting RAW 264.7 cells produced low level TGF-β1 (20.4±10.3 μg/mL) and all treatments did not significantly vary the TGF-β1 level. The CAM treated with NOArg PEA and NOArg-Arg PEAs showed a significantly higher dose-dependent TGF-β1 production (2-3 folds) than the untreated CAM control (71.6±16.4 μg/mL). For example, 1 mg/mL of 2-NOArg-4 PEA, 2-NOArg-4-Arg-4 50/50, 2-NOArg-4-Arg-4 20/80 exhibited 137.2±6.4, 151.5±9.6, and 158.3±23.1 μg/mL TGF-β1 production, respectively in the CAM condition. An increase in the dosage of treatment to 2 mg/mL also led to an increase in TGF-β1 production, e.g., from 137.2±6.4 μg/mL at 1 mg/mL to 225.5±7.2 μg/mL at 2 mg/mL of 2-NOArg-4 PEA treated CAM.

The TGF-β1 level of AAM treated with NOArg PEA or NOArg-Arg coPEAs showed no significant difference from the AAM control. For example, at the 1 mg/mL, 2-NOArg-4 PEA, 2-NOArg-4-Arg-4 50/50, 2-NOArg-4-Arg-4 20/80 and 2-Arg-4 PEA treated AAM produced TGF-β1 at 392.4±15.3, 376.6±15.7, 383.6±10.9, 349.0±15.2 μg/mL level, respectively vs. 418.9±30.9 μg/mL pg/mL of the AAM control. Many studies reported inhibiting NO production were associated with an increased TGF-β1 expression in vitro and in vivo, while Arg supplement could have opposite effect due to elevated NO biosynthesis. For NOS inhibitor treated chondrocytes, one mechanism of the increase in TGF-β1 is because of the decreased NO level, and such a reduced NO level can lead to the loss of its inhibition to IL-1β which stimulates TGF-1 production. Macrophages may have a similar mechanism.

The CAM treated with NOArg PEA, 50/50 PEA and 20/80 PEA produced more TGF-β1 than the untreated CAM control may facilitate the transition of multiple types of cells from pro-inflammatory stage to anti-inflammatory stages. In this in vitro study, these newly designed NOArg PEA and NOArg-Arg coPEA biomaterials treated CAMs (early inflammatory stage wound has more CAMs) have shown lower NO, higher arginase activity and higher TGF-β1. Those changes should facilitate the transition of a pro-inflammatory character in an early stage of wound healing to a pro-healing character after the acute inflammatory phase. TGF-β1 is a potent anti-inflammatory mediator produced by macrophages and fibroblasts in the healing wound, which plays an important role in the development, differentiation, immune function and wound repair. TGF-β1 inhibits iNOS activity by attenuating LPS induced iNOS mRNA in macrophages and increase arginase, ornithine decarboxylase and ornithine aminotransferase activity. The process is an important mechanism involved in the transition of the wound from the inflammatory stage to the proliferative stage of wound healing. TGF-β1 also increases the expression of ED-A fibronectin. In the presence of mechanical stress, TGF-β1 and ED-A promote the modulation of proto-myofibroblasts into differentiated myofibroblasts. Due to the complex and dynamic nature of the wound bed environment in an in vivo would healing case, those positive in vitro data should and were further validated as shown below.

Evaluation of wound healing in vivo and histological analysis.

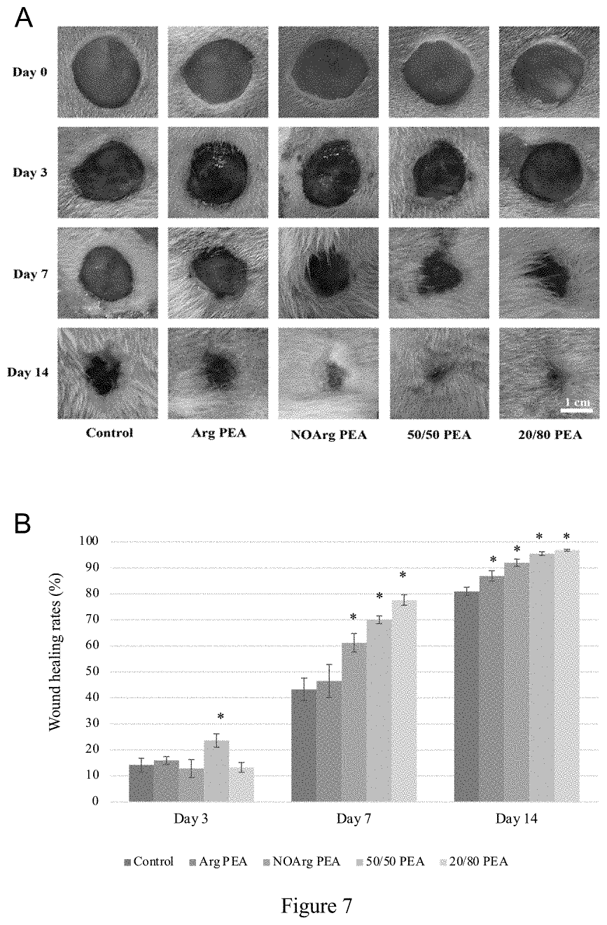

FIG. 7A showed the representative images of the full-thickness wound beds treated with various Arg-based PEAs and their derivatives during the wound healing process of diabetic rat models over 14 days. The image data illustrate that all those wounds with the Arg-based polymer treatments, particularly NOArg PEA, 50/50 PEA and 20/80 PEA, achieved significantly larger reduction in wound area when compared with the control. Such a meaningful wound area reduction becomes apparent as early as day 7, i.e., the wound healing rates were significantly accelerated by NOArg PEA homopolymer, and 50/50 PEA and 20/80 PEA copolymers. FIG. 7B show the quantification of those corresponding wound area reduction image data upon different treatments. The 20/80 PEA group achieved the highest healing rate of almost 80% (nearly double over the control), followed by 50/50 PEA (70%), NOArg PEA (60%), Arg PEA (45%) and control (40%). On day 14, the wounds in the 20/80 PEA and 50/50 PEA groups showed more than 95% healing rate, demonstrating that the design concept of combining both Arg PEA and NOS inhibitor polymeric biomaterials (NOArg PEA) treatments (i.e., 2-NOArg-4-Arg-4 50/50 PEA or 2-NOArg-4-Arg-4 20/80 PEA) significantly accelerated the wound healing process of a diabetic rat model.

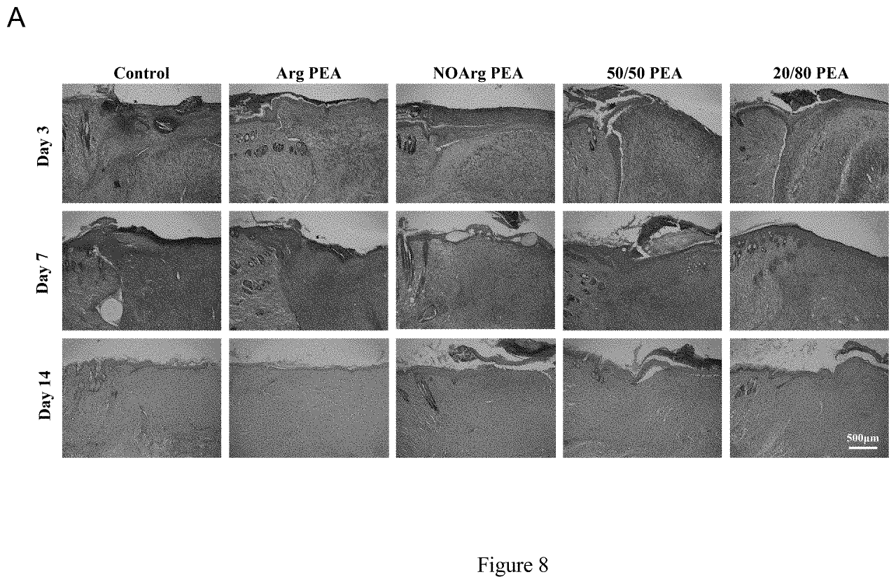

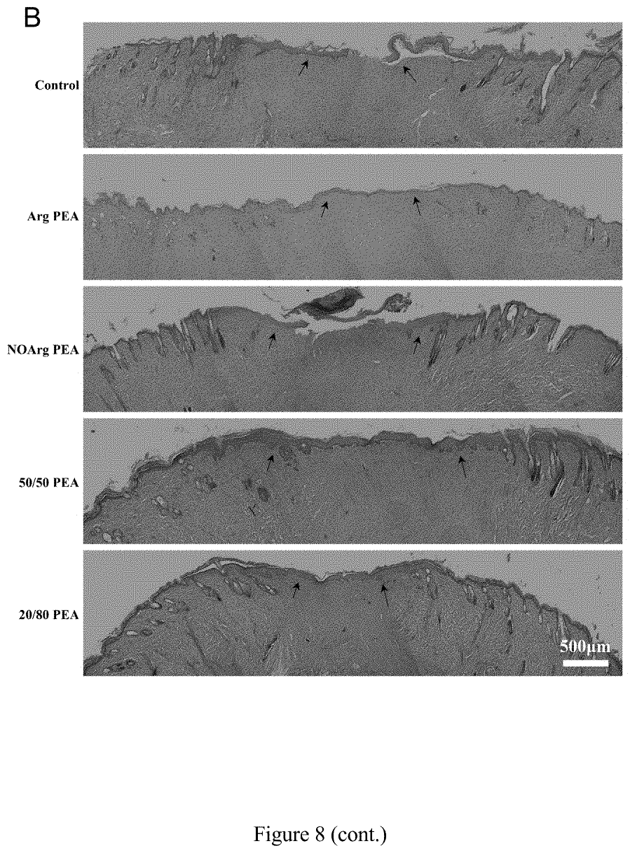

The healing quality of the treated diabetic wounds were assessed for their biological components in the wound beds by H&E staining. As shown in FIG. 8A, the wound beds were filled with fibrin clots in all groups on day 3. The fibrin clot serves as a temporary shield to protect the exposed tissues and provides a provisional matrix through which cells can migrate during the healing process. The fibrin clots also contain a variety of cytokines and growth factors, which can recruit circulating inflammatory cells and initiate the wound healing process. Neutrophils and macrophages, started to infiltrate into the wound beds (dark red region on the right in the images in FIG. 8). The resident dermal fibroblasts in the neighborhood of the wound begin to migrate into the provisional fibrin clot and lay down the collagen rich matrix 3-4 days after wound insult. In FIG. 8A, 7 days after surgery, the control and Arg PEA treated wounds showed more infiltrated inflammatory cells than NOArg PEA, 20/80 PEA and 50/50 PEA treated wounds, indicating lower inflammatory levels or earlier transformation towards proliferative stage of the wound healing process. Meanwhile, keratinocytes started to migrate and formed a typical epidermal tongue at the wound edge in all groups (FIG. 8A). The 20/80 PEA and 50/50 PEA treated wounds showed a more completed formation of neo-epidermis than other treatments and control, i.e., faster re-epithelialization. The NOArg PEA treated wound also showed better re-epithelialization than the Arg PEA treatment and control. In wound healing, re-epithelialization is the critical step for forming a physical barrier against external bacteria and other harmful substances. All wounds except the control were fully covered with neo-epidermis 14 days after surgery (FIG. 8B). The 20/80 PEA and 50/50 PEA treated wounds had thicker layers of neo-epidermis across the entire wound beds, i.e., more epidermal hyperplasia.

The in vitro test data (FIGS. 5 and 6) showed the NOArg-Arg co-PEAs and NOArg PEAs treated CAM produced less NO, more TGF-β1 and a higher arginase activity than the CAM control. These in vitro data are further validated in terms of the actual wound healing outcome in this vivo diabetic wound healing data (FIGS. 7 & 8) in a diabetic mouse model. Therefore, the treatment of wounds by our newly designed tunable immune responsive NOArg-Arg co-PEAs and NOArg PEA biomaterials can facilitate a timely transition of wounds from an inflammatory stage to granulation tissue formation stage that lead to faster wound closure, earlier re-epithelialization and eventual healing. This may have an important implication in those wounds that are difficult to heal like diabetic ulcer wounds.

Immunohistochemical Analysis.

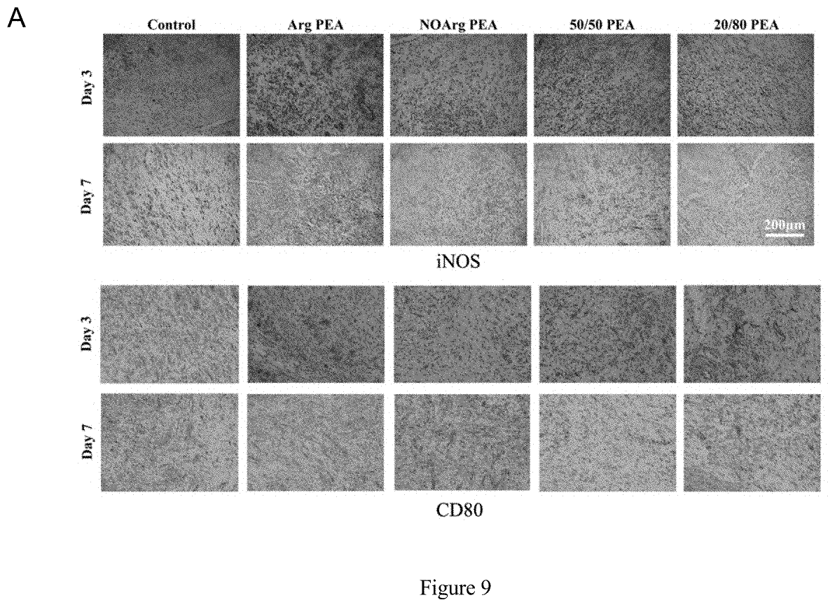

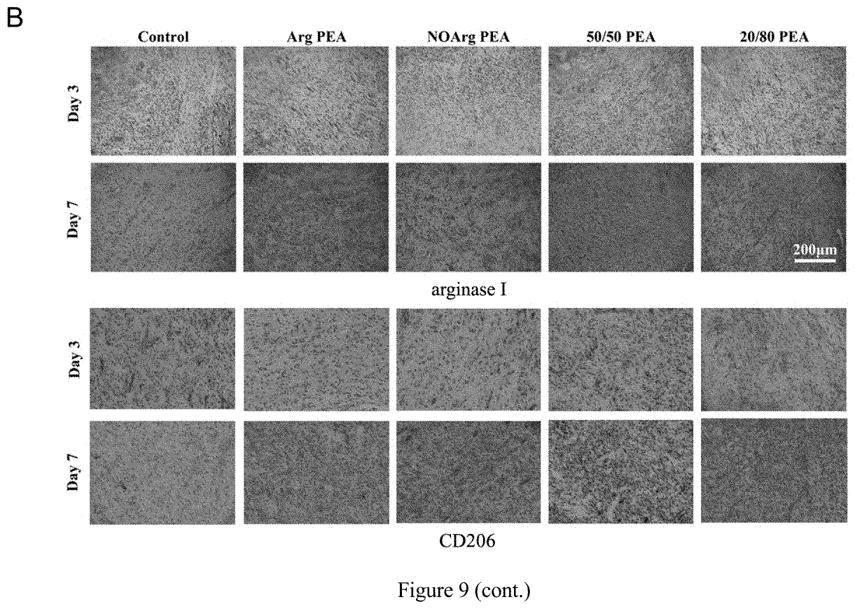

The data from the immunohistochemical analysis of the wound beds for both iNOS expression and arginase activity are given in FIG. 9. As shown in FIG. 9A, on day 3, the iNOS expression in NOArg PEA, 50/50 PEA and 20/80 PEA treated wounds are slightly lower than Arg PEA treated wound and untreated control. On day 7, all wounds showed decreased iNOS level compared to day 3. The iNOS expression of the 20/80 PEA treated wound showed the lowest iNOS level than the untreated control, NOArg PEA and 50/50 PEA treated wounds, while the Arg PEA treated wound showed highest iNOS level; while the 50/50 PEA and NOArg PEA treated wounds had the next lowest iNOS level.

CD80 marker was used to show the proinflammatory cell density (may including neutrophil, monocyte, monocyte derived macrophages on day 3, mainly CAMs on day 7) in wound. On day 3, the Arg PEA, 50/50 PEA, and 20/80 PEA treated wounds showed more infiltrated immune cells with pro-inflammatory phenotype than NOArg PEA treated wounds and untreated control. On day 7, the 50/50 PEA and 20/80 PEA treated wound showed less cells expressing CD80 (mainly CAM) than the untreated control, Arg PEA, and NOArg PEA treated wound. The trend of iNOS expression and CD80 proinflammatory cell density data are consistent on days 3 and 7 indicating that the newly designed NOArg PEA and NOArg-Arg PEAs cannot alter the iNOS expression of individual CAM, but can reduce its level of expression as reflected in a lower level of NO production and other proinflammatory signals shown in in vitro data (FIG. 5A). Therefore, the 50/50 PEA and 20/80 PEA treated wounds showed a lower iNOS expression and less infiltrated CAM at a late inflammatory stage (day 7), suggesting an earlier resolution of pro-inflammatory stage from the NOArg coPEA treatments than the NOArg PEA, Arg PEA treated and untreated control. These in vivo wound healing data also illustrate a critical point of using Arg-based biomaterials to modulate the inflammatory response of a diabetic ulcer wounds: neither a pure Arg-PEA nor a pure NOS inhibitor-based PEA (NOArg PEA) can provide a near optimal and timely transformation of a wound from its acute inflammatory to pro-healing stages due to the dynamic and complexity of a wound healing process. A proper balance of both a pure Arg and NOS inhibitor composition in the copolymeric biomaterials is required for achieving a near optimal wound healing in vivo.

In FIG. 9B, the 50/50 PEA and 20/80 PEA treated wounds also showed more arginase I expression on both days 3 and 7. On day 7, all Arg-based PEA treated wounds showed higher AAM marker and CD 206 (mannose receptor), than the untreated wound. The CD206 is a biomarker for M2 macrophage. Thus, a combination of arginase I and CD206 biomarker data on Day 7 clearly demonstrates the molecular and cellular base behind the observed faster wound healing rate and better wound healing quality (collagen contents and re-epithelialization level). The NOArg PEA, 50/50 PEA and 20/80 treated wounds showed slightly higher AAM densities than the Arg PEA treated one.

Therefore, at both the molecular and cellular data, both 50/50 PEA and 20/80 PEA copolymers showed least pro-inflammatory and most pro-healing wound microenvironment than the untreated control and pure Arg supplemental treatment (Arg PEA) or pure iNOS inhibitory treatment (NOArg PEA). The earlier transition of the wound from pro-inflammatory stage to pro-healing stage should mainly be attributed to the capability of the NOArg-Arg coPEAs to regulate the functions of macrophages, particularly their Arg metabolic pathways. The resolution of prolonged inflammatory stage can improve the microenvironment of wound beds, leading to better healing of chronic wounds (FIG. 7 in vivo wound healing data).

Collagen Deposition Evaluation.

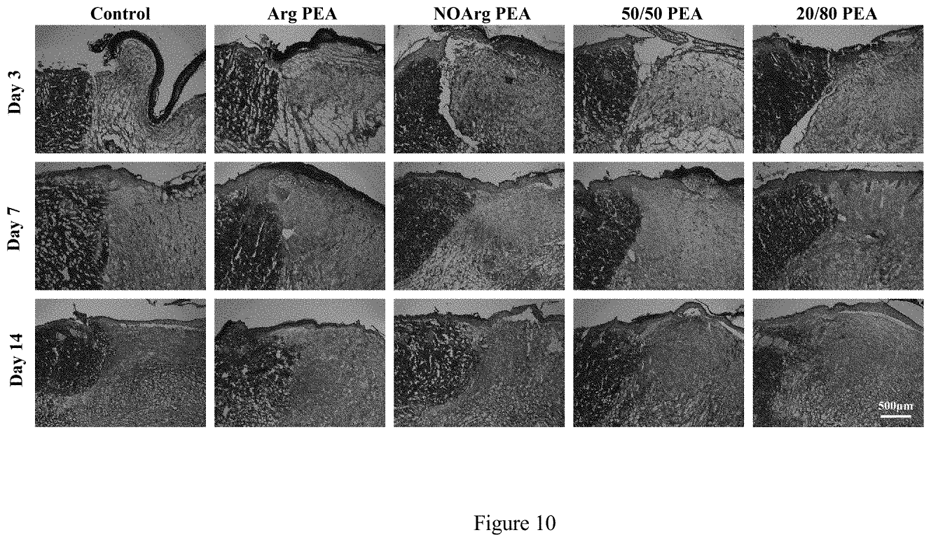

The level of newly synthesized collagen in the regenerated tissues upon the treatments by the NOArg-containing biomaterials is shown in FIG. 10. On days 3 and 7, a limited deposition of collagen was observed in the wound beds of all groups (blue fibrous structure on the right) as the wounds were still in the inflammatory stage. When the inflammatory stage ended on day 14, the wounds treated by the 50/50 PEA showed the highest level of newly synthesized collagen contents with a densely packed fibrous structure, and the 20/80 PEA also showed slightly higher collagen contents than the NOArg PEA and Arg PEA treated wound beds and control groups. The pure Arg PEA treatment did not alter the collagen production level from the control, even though the Arg was provided to the wound milieu via Arg PEA biomaterial, probably because a lot of Arg was largely consumed by macrophages via iNOS pathway. Increased collagen production in the 50/50 PEA or 20/80 PEA treatment groups should be related to more recruited fibroblasts and differentiated myofibroblasts in the more anti-inflammatory microenvironment treated with NOArg-Arg co-PEAs.

Conclusions.

A new family platform of water soluble and biodegradable Arg-based pseudo-protein biomaterials (NOArg PEA and NOArg-Arg co-PEAs) were designed and synthesized for the purpose of providing tunable immune responsive biomaterials to modulate the inflammatory response of wounds for a faster and better wound healing, particularly in those challenging wound healing conditions like diabetic wounds. This new family of Arg-based pseudo protein biomaterials have both Arg and NOS inhibitor (NOArg) building blocks at predetermined composition ratios. These new biomaterials were tested under two different means of activating macrophage conditions, classically activated by LPS+IFN-γ (CAM) and alternatively activated by IL-4 (AAM). These new biodegradable biomaterials can be chemically tuned for achieving different levels of NO, cytokine and growth factors production under both CAM and AAM conditions. NOArg PEA and NOArg-Arg coPEAs were found noncytotoxic toward murine macrophages and fibroblasts. These new biomaterials reduced the NO production of the CAM, increased the arginase activity in both CAM/AAM, increased TGF-β1 production of CAM, and had no significant effect on TNF-α production. The diabetic rats with full thickness incisional wounds treated with NOArg PEA and NOArg-Arg PEAs showed faster wound healing rate than the untreated control on days 7 and 14. The 2-NOArg-4-Arg-4 20/80 and 2-NOArg-4-Arg-4 50/50 treatments led to a more pro-healing wound microenvironment and improved re-epithelialization rate during the first 7 days. The 2-NOArg-4-Arg-4 20/80, 2-NOArg-4-Arg-4 50/50 treatments also slightly increased the collagen deposition in the wound beds. Therefore, the treatment of wounds by our newly designed tunable immune responsive NOArg-Arg co-PEAs and NOArg PEA biomaterials can facilitate the timely transition of wound microenvironment from an inflammatory stage to granulation tissue formation stage that lead to faster wound closure, earlier re-epithelialization and eventual healing. This new approach may provide a viable alternative to advance the treatment of wounds that are difficult to heal like diabetic wounds.

### Example 2

This example provides a description of polymers of the present disclosure and uses thereof.

For the purpose of modulating the microenvironment of impaired wound healing, a series of biodegradable L-NOArg and Arg PEA copolymer (co-PEA) were designed and synthesized in this study using a similar methodology in our prior studies of biodegradable and biocompatible Arg-based polyester amide and poly(ester urea urethane) (PEUU). As the new PEA materials degrading in a wound milieu, the released Arg prevent the depletion of Arg in the wound milieu (maintaining a proper NO level and preventing the production of ROS/RNS from iNOS pathway), while the released NOS inhibitors suppress the overproduction of NO. The rat model of diabetic wound was used to demonstrate the feasibility and efficacy of NOArg-Arg co-PEA as a topical treatment to the chronic wound.

Experimental.

Materials.

L-Arginine hydrochloride (L-Arg-Cl) (Alfa Aesar, Ward Hill, Mass.), NOArg (Chem-impex int'l inc, IL), 1,4-butanediol (Alfa Aesar, Ward Hill, Mass.), triethylamine (TEA) (99%, EMD Chemical, Darmstadt, Germany), sodium nitrate (VWR Science, West Chester, Pa.), p-toluene sulfonic acid monohydrate (TsOH.H₂O) and p-nitrophenol (JT Baker, Philipsburg, N.J.) were used for the polymer synthesis. Solvents including N, N-dimethylacetamide (DMAc) (Beantown Chemical, Hudson, N.H.), isopropyl alcohol (ACS, 99.5%, Macron Chemicals, Philipsburg, N.J.), acetone, dimethyl sulfoxide (DMSO) (Mallinckrodt, St. Louis, Mo.), ethyl acetate (BDH, London, UK), ethanol, benzene, toluene and chloroform (VWR Science, West Chester, Pa.) were used without further purification. Molecular weight cutoff (MWCO) 1,000 g/mol and 3,500 g/mol snakeskin dialysis tubes were purchased from Thermo Fisher Scientific, Rockford, Ill. Bromine (VWR Science, West Chester, Pa.), NaOH (Macron Chemicals, Philipsburg, N.J.), α-naphthol (Beantown Chemical, Hudson, N.H.), urea (Fisher Scientific, Rockford, Ill.), α-isonitrosopropiophenone (Acros Organics, Geel, Belgium) and Griess reagent (Enzo Life Science, Farmingdale, N.Y.) were used in Sakaguchi test, NO production and arginase activity assays. L-NAME was purchased from Alfa Aesar, Ward Hill, Mass.

Synthesis of Dihydrochloride Acid Salt of Bis (NOArg) Butane Diester Monomers (NOArg-4) and Bis (Arg) Butane Diester Monomers (Arg-4-Cl).

The NOArg-4 monomer was synthesized using a previously described but a modified method (i.e., in refluxing benzene instead of toluene). Briefly, NOArg (0.04 mol), 1,4 butanediol (0.018 mol), TsOH.H₂O (0.05 mol) and sodium nitrate (15 gram) were mixed well with 25 mL of water in a flask. 150 mL of benzene (b.p. 80° C.) was added and the heterogeneous solid-liquid reaction mixture was heated in an oil bath (87-90° C.) and refluxed for 36 h. All water in the reaction was collected by a dean-stark apparatus. After the reaction was completed and cooled to room temperature, benzene was decanted. The product was dissolved in 120 mL of ethanol and the insoluble sodium nitrate was filtered. The crude NOArg-4 monomer was collected from the ethanol solution using a rotary evaporator (Buchi, R110), then purified by recrystallization three times in cold isopropyl alcohol (precipitate at −20° C.) and vacuum-dried at room temperature overnight. Arg-4-Cl monomer was synthesized using the method described in the prior studies of the Arg-based PEAs and PEUUs with detailed procedures.

Synthesis of the NOArg-PEA Homopolymer and NOArg-Arg Co-PEAs

Both homopolymers (2-NOArg-4 PEA, 2-Arg-4 PEA) and copolymers (2-NOArg-4-Arg-4 50/50-PEA, 2-NOArg-4-Arg-4 20/80-PEA) were synthesized by a solution polycondensation of Arg-4-Cl, NOArg-4, and p-nitrophenol diester monomers via prior published procedures. Briefly, the polymerization was carried out in DMAc solution in an oil bath at 70° C. for 30 h and triethylamine was used as the acid receptor for TsOH. The stoichiometric ratio of amino acid diester (NOArg-4 and Arg-4-Cl monomers in total) top-nitrophenol diester is 1:1. The resulting NOArg-Arg co-PEAs are generically labeled as y-NOArg-x1-Arg-x2, where y, x1 and x2 are the numbers of CH₂ groups in diacid and diol segments, respectively (FIG. 1). The abbreviated sample labels are used in the following discussion and listed in Table 2. All PEA samples were carefully purified to remove chemical residuals and possible stimulants to the immune cells. After the polymerization, the PEA solutions in DMAc were poured and precipitated in acetone to remove the most of the p-nitrophenol, triethylamine, etc. The precipitated PEAs were filtered, vacuum-dried, dissolved in Milli-Q water and further purified by dialysis against Milli-Q water at 4° C. in autoclaved glass beakers (3 L, 2 h, MWCO: 3,500 g/mol). The purified sample solution in dialysis tubes was transferred to sterilized centrifuge tubes, lyophilized and stored at 4° C.

Characterizations of NOArg-4, NOArg-PEAs and NOArg-Arg co-PEAs

The NOArg-4 and NOArg-Arg co-PEA series were analyzed by proton nuclear magnetic resonance (¹H-NMR). ¹H-NMR spectra were recorded on a Varian (Palo Alto, Calif.) INOVA-400 spectrometer at 400 MHz. Deuterated dimethyl sulfoxide (DMSO-d6, Cambridge Isotope Laboratories) was used as the solvent. The sample concentration in DMSO-d6 was about 1% (w/v). All of the chemical shifts were reported in parts per million (ppm). Fourier transform infrared (FTIR) spectra of the NOArg-4 and NOArg-Arg co-PEA series were recorded on a PerkinElmer (Madison, Wis.) Nicolet Magna 560 FTIR spectrometer with Omnic software for data acquisition and analysis. All solubility tests were performed in glass vials with magnetic stirring at room temperature. Gel permeation chromatography (GPC) of PEA series was done on a Waters aqueous GPC equipped with a Waters 410 differential refractive index detector. The injection volume of the polymer sample solution (5 mg/mL in Milli-Q water) was 100 μL. Polyethylene oxide standard was used for calibration.

In Vitro Degradation of NOArg-PEA or NOArg-Arg co-PEAs by Sakaguchi Test,

The degradation of NOArg-PEA and NOArg-Arg co-PEA series were evaluated quantitatively by the Sakaguchi test. 20 mg of each sample (NOArg-PEA, 20/80-PEA, 50/50-PEA) was dissolved in 5 mL of PBS at room temperature then loaded in dialysis tubes (10 mm diameter, MWCO: 1,000 g/mol). Dialysis tubes were immersed in 95 mL of a 0.1 M pH 7.4 PBS and incubated at 20° C. and 37° C. At predetermined intervals, 1 mL of immersion PBS was sampled at predetermined intervals and 1 mL of fresh PBS was added to compensate the immersion solution volume. The 1 mL of the immersion PBS sample solution was diluted with DI water to 5 mL in test tubes and cooled on ice. 1 mL of a 10% NaOH solution and 1 mL of a 0.02% α-naphthol ethanol solution were added and mixed in test tubes embedded in ice. After 1 h, 200 μL of a hypobromite solution (prepared by dissolving 2 g of bromine in 100 mL of 5% NaOH, which has been cooled) was added with shaking, then 1 mL of a 40% urea solution was added to each test tube. The absorbance of the solutions at 515 nm was tested using a UV-Vis spectrophotometry (PerkinElmer Lambada 35, Madison, Wis.). A calibration curve of Arg concentration can be established by assaying 5 mL of an Arg solution in DI water with a linear range (2-30 μg/mL). Sakaguchi test is usually considered to be specific to the presence of the guanidino group of Arg in solutions or in proteins. However, the water-soluble derivatives of NOArg, such as L-NAME, has a lower absorbance at 515 nm than the free Arg (about 26% absorbance at the equal molar concentration of Arg). So, the data of degraded NOArg-PEA and NOArg-Arg co-PEAs in PBS were normalized against that of the completely hydrolyzed samples. Briefly, 2 mg of NOArg-Arg co-PEA samples were hydrolyzed in 1 mL of a 0.2 M NaOH solution at 40 C.° for 2 h, vacuum dried, dissolved in 20 mL of DI water, diluted to 5 mL with DI water and assayed. The average of 3 samples of each type of the PEAs were considered as the absorbance at 100% degradation.

Cytotoxicity of NOArg-4, NOArg-PEA and NOArg-Arg co-PEAs

3T3 fibroblasts were maintained in Dulbecco's Modified Eagle Medium (DMEM) supplemented with 10% fetal bovine serum (FBS, Mediatech, VA) and 100 U/mL penicillin-streptomycin. RAW 264.7 cells were maintained in DMEM supplemented with 10% de-lipidized FBS (endotoxin <0.3 EU/mL, Gemini Bio-products, CA), 1% 1 M 4-(2-hydroxyethyl)-1-piperazineethanesulfonic acid (HEPES) buffer, 1% 100 mM sodium pyruvate, 1% 200 mM L-glutamine and 100 U/mL penicillin-streptomycin.

The cytotoxicity of NOArg-4 and PEA series against RAW 264.7 cells and 3T3 fibroblasts was evaluated by a standard MTT assay. Cells were seeded in 96-well plates at a density of 8×10³ cells/well. All NOArg-4 and PEA samples were dissolved in complete DMEM media and filtered through 0.22 μm filters (Pall Corporation, NY). 1 mg/mL or 2 mg/mL NOArg-4 and PEA series in DMEM supplemented with 10% FBS were incubated along with RAW 264.7 or 3T3 cells for 24 and 48 h. Cells cultured in media were used as controls. After incubation, the media was removed, washed with PBS, and 100 μL of media containing 10 μL of 5 mg/mL MTT solution (thiazolyl blue tetrazolium bromide in PBS filtered by a 0.22 μm filter) was added to each well following another 4 h of incubation. 150 L of DMSO was added to each well after removing the MTT media, and the 96-well plate was gently shaken for 30 min at room temperature. The absorbance of the DMSO solution was measured at wavelengths of 570 and 690 nm (Spectramax plus 384, Molecular Devices, U.S.A.). The cell viability (%) was calculated according to the following equation:

$$Viability ( \% ) = \frac{OD 570 ( sample ) - OD 690 ( sample )}{OD 570 ( control ) - OD 690 ( control )} \times 1 \; 00 \%$$

RAW 264.7 cells and 3T3 fibroblasts were seeded in 12-well cell culture plates with the density of 1.5×10⁵ per well for a live and dead cell viability assay. Cells were incubated with 2 mg/mL NOArg-PEA, 50/50-PEA and Arg-PEA in complete media for 24 h. After washing with PBS, the cells were stained with calcein AM (Biovision, CA) and propidium iodide (PI, Life technologies, CA). Images were taken after staining for 5 min.

NO production and arginase activity assay of RAW 264.7 cells with treatments

4×10⁵ RAW 264.7 cells in each well were incubated in 400 μL of complete DMEM in 24 well plates for 24 h. For the resting macrophages group, media was then removed and replaced with treatments of 1 mg/mL or 2 mg/mL Arg, L-NAME, Arg+L-NAME (1:1 weight ratio mixed), NOArg-4 monomer, NOArg-PEA, 50/50-PEA, 20/80-PEA or Arg-PEA in DMEM supplemented with 5% FBS. To induce different macrophage activation phenotypes, treatments contained either 100 U/mL recombinant mouse interferon-γ (IFN-γ, 1.25×10⁷ U/mg, Shenandoah Biotechnology, PA) plus 100 ng/mL lipopolysaccharide (LPS, Sigma-Aldrich) for the CAM or 20 ng/mL recombinant mouse interleukin-4 (IL-4, Shenandoah Biotechnology, PA) for the AAM, respectively. Resting Macrophages, CAM and AAM incubated in DMEM contains 5% FBS without treatments were used as controls.

After 24 h incubation, the NO production in the supernatant media was measured by Griess Reagent according to the manufacturer's protocol (Enzo life Science, NY). The absorbance at 540 nm was measured within 30 min in a plate reader (Spectramax plus 384, Molecular Devices, U.S.A.). The arginase activity assay was tested colorimetrically. The cells were washed with PBS and lysed by 200 μL of 0.2% Triton X-100 in 20 mM Tris-HCl (pH 7.5) containing protease inhibitors (20 μg pepstatin A, 20 μg aprotinin and 20 μg leupeptin). 200 μL of a 10 mM MnCl₂ solution was added to the lysate and the enzyme was activated for 10 minutes at 55° C. 100 μL aliquot of the activated lysate was mixed with 100 μL of a 0.5 M Arg solution and incubated at 37° C. for 1 h. The reaction was stopped by the addition of 800 μL of mixed acid solution (H₂SO₄/H₃PO₄/H₂O=1/3/7) and 100 μL of a 9 wt % α-isonitrosopropiophenone in ethanol solution. The mixture solution was heated in a 95° C. water bath for 45 min. After being cooled to room temperature, the absorbance of 200 μL of the mixture solution at 540 nm was tested in 96 well plates. A urea calibration curve was prepared with increasing amounts of urea between 1.5 and 30 μg. One unit (U) of arginase activity was defined as the enzyme activity that catalyzes the production of 1 μM of urea per minute under the condition of the assay. The NO production and the arginase activity in three replicate wells of each condition were assayed and the mean value was calculated with standard deviations.

TNF-α and TGF-β1 Production of RAW 264.7 Cells with Treatments.

The cell culture and treatment of 4×10⁵ RAW 264.7 cells were as described in the above section. TNF-α and TGF-β1 production were measured by ELISA according to the manufacturer's instructions (mouse TNF-α standard TMB Elisa kit, Peprotech, NJ and TMB substrate set, Biolegend, CA were used for the TNF-α assay and TGF beta 1 ELISA kit, Affymetrix, CA was used for the TGF-β1 assay). Three replicate wells were assayed, and the mean value was calculated with a standard deviation.

In vivo wound healing study in diabetic rat models.

Induction of Type I Diabetic Rat Model and Surgical Procedures.

250 to 300 g weighed Sprague-Dawley (SD) rats were obtained from the Laboratory Animal Center, Guangdong Pharmaceutical University. All rats were housed and fed in a specific pathogen-free (SPF) environment during the study. All animal procedures were approved by the institutional animal care and use committee. Diabetes was induced by intraperitoneally injecting 1% streptozotocin (STZ, 65 mg/kg) dissolved in 0.1 M sodium citrate buffer. This method can cause hyper-glycemia (>16.7 mM) in rats for at least 120 days. After 7 days, if the fasting blood glucose level was higher than 16.7 mM, together with the observations of weight loss, polyuria, and polydipsia, rats were considered diabetic rat models. This STZ-induced rat model has been widely used to study the pathogenesis of type 1 diabetes mellitus (T1DM), as well as to evaluate anti-diabetic agents.

Each of these materials (NOArg-PEA, Arg-PEA, and NOArg-Arg co-PEA) were mixed with a 30% w/v Pluronic F-127 solution. Pluronic F-127 gels containing 10 mg/mL various PEAs were used as the treatment formulations. The diabetic rats were anesthetized using 10% chloral hydrate (300 mg/kg body weight). After the dorsal hair of rats was shaved and sterilized, two circular, full-thickness skin wounds with the diameter of 18 mm were created on the back of each rat. Rats were then randomized into five groups (n=4) and treated with Arg-PEA, NOArg-PEA, 20/80-PEA, 50/50-PEA gels or Pluronic F-127 gel (control). Finally, all wounds and surrounding areas were covered with 3M Tegaderm™ (3M Health Care, USA). The treated rats were fed in individual cages.

Wound Healing Assessment.

The healing status in each group was evaluated by the images of the wound area on days 0, 3, 7, and 14 post-surgery. Wound areas were calculated using image analysis software Image-Pro Plus (Media Cybernetics, USA). The degree of healing was expressed as the wound healing rate: (A₀−Aₜ)/A₀×100%, where A₀ is the initial wound area (t=0) and Aₜ is the wound area at time t.

Histological and Immunohistochemical Analysis.

The quality of the wound area during healing was assessed by both histological and immunohistochemical data. Rats were sacrificed at 3, 7, and 14 days post-surgery, and the wounded tissue with its surrounding rim of intact skin together was harvested and fixed in 4% paraformaldehyde followed by standard dehydration with gradient ethanol and embedded in paraffin wax. Subsequently, skin tissues were cut into 5 μm sections for both histological and immunohistochemical evaluations. For histological analysis, hematoxylin and eosin (H&E) staining for cell types and contents and Masson's Trichrome staining for collagen contents were performed. For the immunohistochemical staining, sections were deparaffinized, rehydrated and incubated with primary antibodies against arginase I (Arg I sc-18354, Santa Cruz sc-18354, USA, 1:100), nitric oxide synthase-2 (NOS2, Santa Cruz sc-651, USA, 1:100), CD80 (Bioss 2211R, China, 1:500), CD206 (Bioss 4727R, China, 1:500) and CD31 (Abcam, USA, 1:100) overnight at 4° C. Then they were incubated with the streptavidin-HRP secondary antibody (DAKO, USA) for 1 hour. Immunodetection was visualized by a diaminobenzidine (DAB) detection kit (Servicebio, China). The stained slides were imaged by a light microscope (Axioskop 40 FL, Zeiss, Germany) and analyzed using imageJ software. The percentage of positive pixels was graded as follows: Negative=0; ≤10%=1; 11≤50%=2, 51-80%=3; ≥81%=4.

An intensity score was assigned to strong positive pixels, positive pixels and weak positive pixels using the following formula:

$$Score = \frac{( Number of pixels in a zone ) \times ( Score of the zone )}{Total number of pixels in the image}$$

The score of the zone was 4 for strong positive pixels, 3 for positive pixels and 2 for weak positive pixels. The scores for each zone were summed to arrive at the intensity of positive pixels. An immunoreactivity (IR) score was calculated by multiplying the intensity score of the positive pixels by the grade of the proportion of positive pixels in the image.

Statistical analysis.

The in vitro cell based assay and in vivo animal studies results are shown in column graphs as mean±standard deviation. For the in vitro studies (i.e. NO production, arginase activity, TNF-α, TGF-β1), the level of significant differences between each treatment group and control group was determined by the two-tailed t test. The number of asterisks or pound signs in the figures indicates the statistical significance as follows: *, p<0.05; **, p<0.01. For the quantitative data of in vivo studies (i.e. wounding healing rate, collagen content, and blood vessel density), a one-way ANOVA followed by a Bonferroni multiple comparison test was carried out. Animal number and number of times experiments were repeated are indicated in the figure legend.

Results and Discussion.

Synthesis of NOArg-4 Monomers, NOArg-PEA Homopolymers, and NOArg-Arg Co-PEA Copolymers.

The NOArg-4 monomers were synthesized from NOArg and 1,4-butanediol in refluxing benzene (FIG. 1A). NOArg can be considered as an N^{ω}-nitro protected Arg derivative and the removal of the nitro group (deprotection) usually requires catalysts and strong acids. However, the reaction in refluxing toluene (b.p. 110° C.) which is used for most esterification of amino acids could lead to a partial removal of the nitro group (tested by FTIR and Sakaguchi test). The esterification of NOArg and 1,4-butanediol in refluxing benzene (b.p. 80° C.) prevented the removal of the nitro group. Also, the reactants were mixed with sodium nitrate (insoluble in benzene) to prevent locally overheating when the viscosity of the reaction increased in the esterification process.

TABLE 2
Molecular weight of NOArg-PEA, Arg-PEA, and NOArg-Arg co-PEA

| | Monomers molar | | | | |
|---|---|---|---|---|---|
| | feed ratio | | | | |
| | (NOArg-4:Arg-4:di- | | | | |
| | mtrophenol | Sample | Mₙ | M_{w} | |
| | succinate) | Label | (g/mol) | (g/mol) | PDI |
| 2-NOArg-4 | 10:0:10 | NOArg-PEA | 18,800 | 26,700 | 1.42 |
| 2-NOArg-4-Arg-4 | 5:5:10 | 50/50-PEA | 19,700 | 29,100 | 1.48 |
| 50/50 | | | | | |
| 2-NOArg-4-Arg-4 | 2:8:10 | 20/80-PEA | 21,600 | 31,400 | 1.45 |
| 20/80 | | | | | |
| 2-Arg-4 | 0:10:10 | Arg-PEA | 19,800 | 27,100 | 1.37 |

Chemical and Material Characterizations

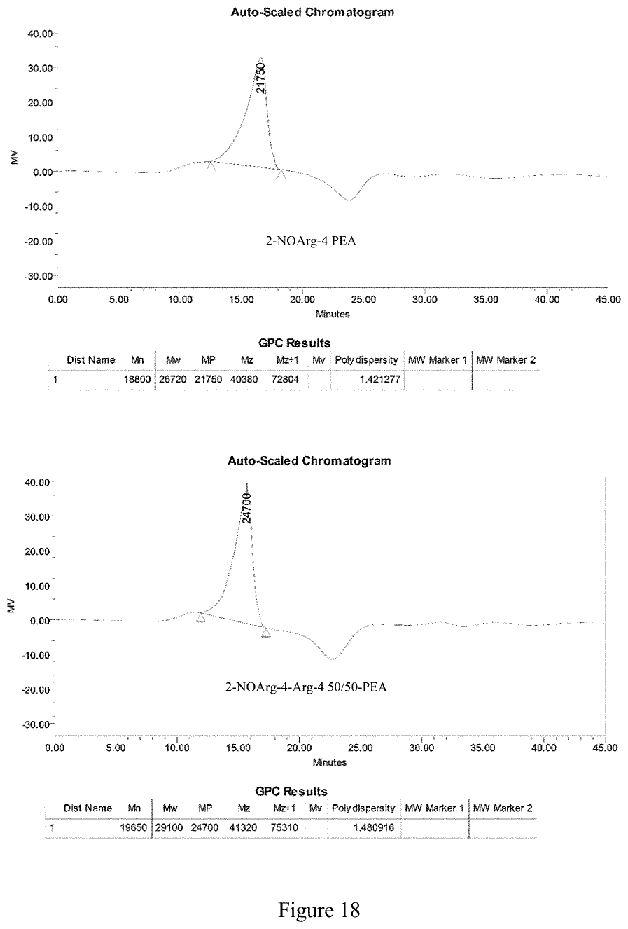

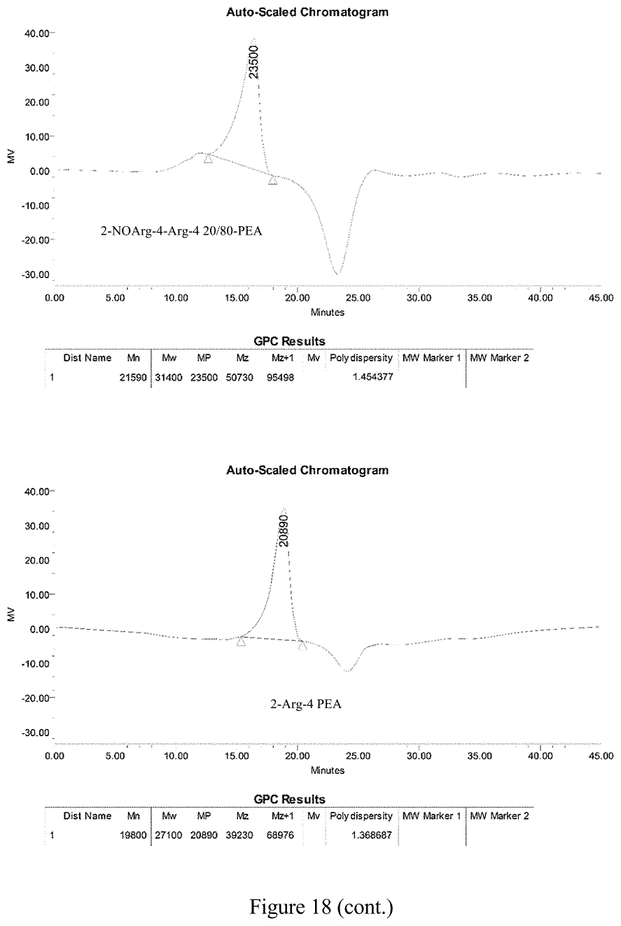

The chemical structure of NOArg-4 was characterized by FT-IR and H-NMR (FIG. 18). Tetra-p-toluene sulfonic acid salt of bis (L-NOArg) butane diester (NOArg-4): FT-IR (cm⁻¹): 1740 [—C(O)—], 1590 [—NO₂]. ¹H-NMR (DMSO-d6, ppm, δ): 1.55 [4H, —OC(O)—CH(NH₃⁺)—CH₂—CH₂—CH₂—NH—], 1.72 [4H, —(O)CO—O—CH₂—CH₂—], 2.29 [12H, H₃C-Ph-SO₃—], 2.55 [—CH₂—CH₂—CH₂—NH—], 3.10 [4H, —(O)C—O—CH₂—CH₂—], 3.90 [2H, H₂N—CH(R)—C(O)—O—], 8.22 [4H, H₂N—CH(R)—C—(O)—O-] (supplemental data, FIG. 18C). The synthesis and chemical characterization of Arg-4-Cl monomer and the di-nitrophenyl succinate monomer used in the copolymerization had been described in prior studies. ¹H-NMR spectra of NOArg-4 and Arg-4 diester are very similar because the nitroguanidine group of NOArg does not contain hydrogen atoms that have very different chemical environment compared to the guanidino group of Arg. Therefore, ¹H-NMR is not able to be used to quantify the ratio of NOArg and Arg in the copolymers. NOArg-4 monomer and L-NAME showed the absorbance of ester bond at 1,740 cm⁻¹ and the asymmetric vibration of NO₂ (vₐₛ (NO₂)) absorbance at 1590 cm⁻¹ (supplemental data, FIG. 18A). NOArg-4 monomer synthesized in toluene showed fewer vₐₛ (NO₂) absorbance than NOArg-4 synthesized in benzene, suggesting the nitro group on the guanidine could be partially removed in refluxing toluene. FTIR spectra of synthesized PEAs clearly shows the absorbance of the amide I and amide II bond formed in the polymerization at 1,640 cm⁻¹ and 1,550 cm⁻¹ (FIG. 18B). As the NOArg content increases in the copolymer, the vₐₛ (NO₂) absorbance of 50/50-PEA and NOArg-PEA also presents at 1,590 cm⁻¹ on the spectra.

Similar to L-NAME, the carboxyl group of NOArg forms ester bond with the hydroxyl groups of 1,4-butanediol in NOArg-4 monomer, NOArg-PEA, and NOArg-Arg co-PEAs, hence all showed increased water solubility. As the composition of NOArg in the copolymer increases, the solubility in water becomes lower. NOArg-PEA, Arg-PEA, and NOArg-Arg co-PEAs could also be dissolved in DMAc, DMF, and DMSO (>5 mg/mL), but are insoluble in acetone, chloroform, dichloromethane, tetrahydrofuran, methanol, ethanol etc. The number average molecular weight (Mₙ) of NOArg-PEA and NOArg-Arg co-PEA ranged from 18,800 to 21,600 g/mol (Table 2, FIG. 18D), similar to the Mₙ of the prior synthesized Arg-PEA homopolymer.

NOArg-PEA and NOArg-Arg Co-PEA Degradation In Vitro.

The level of hydrolytic degradation of NOArg-PEA and NOArg-Arg co-PEA is shown in FIG. 11. In general, higher NOArg composition/lower Arg composition in co-PEA achieved slower degradation rate and are degraded faster at a higher temperature, 37° C. vs. 20° C. NOArg-PEA, 50/50-PEA, 20/80-PEA achieved 44.6%, 57.7% and 60.0% degradation at 24 h, and 63.7%, 80.1% and 82.7% degradation at 96 h, respectively at 37° C.

Sakaguchi test was used to provide an estimation of the amounts of NOArg and Arg released from the biomaterials in the process of degradation in a physiological environment. GPC test was not applicable in this degradation study because (1) the degradation products of PEAs (may contains arginine and NOArg) and oligomers cannot be dissolved in most commonly used GPC solvents, such as THF or DMF; (2) retrieving degraded polymers from aqueous buffer media is difficult. The inhibitory effect of PEAs on iNOS activity depends on the available concentration of the small molecule NOArg derivatives and free Arg released in the degradation process. 50/50-PEA and 20/80-PEA achieved a higher than 70% degradation in vitro at 37° C. on day 4. The degradation rate of these PEAs in vivo is probably faster than in vitro because of the presence of proteases within the wound milieu. The differentiation and migration of macrophage in wound tissue also take about 3 days after the injury happens that matches the required degradation time of PEAs.

Arg is the only amino acid whose concentration in a wound fluid decreased over time to undetectable levels. Most Arg catabolism through iNOS is conducted by macrophages that present in a wound site about 3 days after injury and dominate about 7-12 days in a normal healing process. Protein breakdown does not contribute to Arg and NO production. Dietary Arg supplementation has shown beneficial effects on acute incisional wounds. However, no evidence shows dietary supplemental Arg improved chronic wounds (e.g., pressure ulcer, diabetic ulcer). Local early Arg supplementation (infusion or topical application) may disturb the reciprocal regulation of iNOS and arginase, leading to the preferential metabolism of Arg to excess NO production with a consequent reduction in angiogenesis and granulation tissue formation. An excessive amount of NO is inhibitory to the wound healing, particularly in the granulation tissue formation stage. High levels of NO produced by iNOS interacts with oxidative molecules (superoxide is the main component) derived from neutrophils and macrophages (via the phagocytic isoforms of NADPH oxidase) to produce peroxynitrite which induces apoptosis/necrosis of fibroblasts and many other types of cells in the healing process. The supplement of Arg and NOS inhibitors to wound in situ could influence the healing process. When Arg and L-NAME were topically applied together, the hydroxyproline synthesis of the wound tissue was elevated.

Cytotoxicity Test of NOArg-PEA and NOArg-Arg co-PEA.

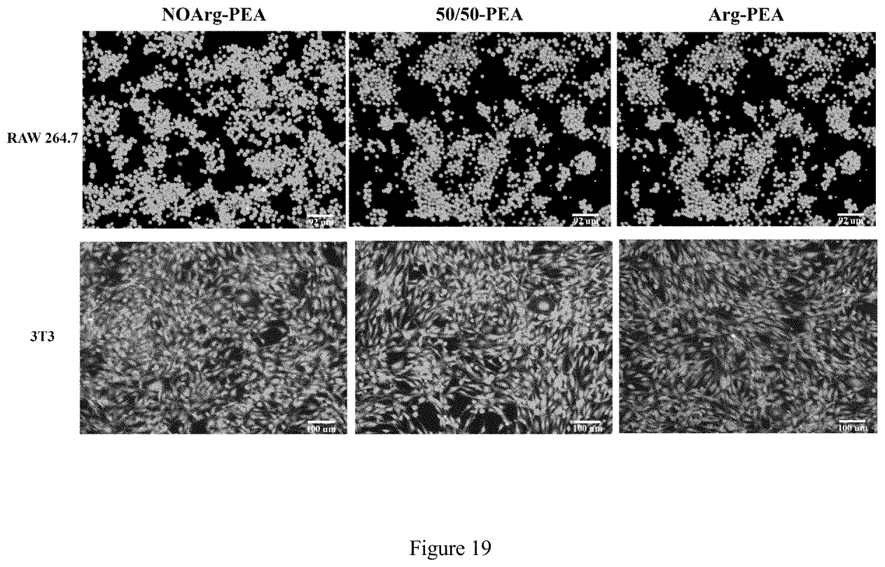

As shown in FIG. 4, irrespective of monomer vs. polymers, copolymer composition, and cell types, all testing samples (NOArg-4 monomer, NOArg-PEA, and NOArg-Arg co-PEA, up to 2 mg/mL concentration) showed no statistically significant cytotoxicity toward RAW 264.7 macrophages and 3T3 fibroblasts compared to the untreated control in 24 h and 48 h. Live/dead assay did not detected cytotoxicity of PEAs as well (FIG. 19)

Macrophages are prominent inflammatory cells which have many functions in wound healing. Fibroblasts and myofibroblasts produce provisional matrix and a local contraction of the matrix within the granulation tissue of healing wounds to reconstitute the tissue continuity. The NOArg-PEA and NOArg-Arg co-PEAs do not influence the viability of both these two important cell types in wound healing. Hence, 1 mg/mL and 2 mg/mL of AA-PEA samples were used in the following in vitro studies.

NO Production and Arginase Activity of RAW 264.7 Cells with Treatments

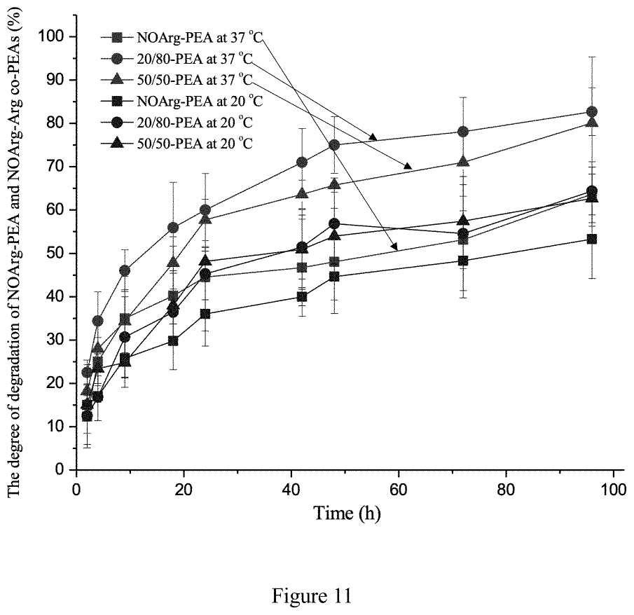

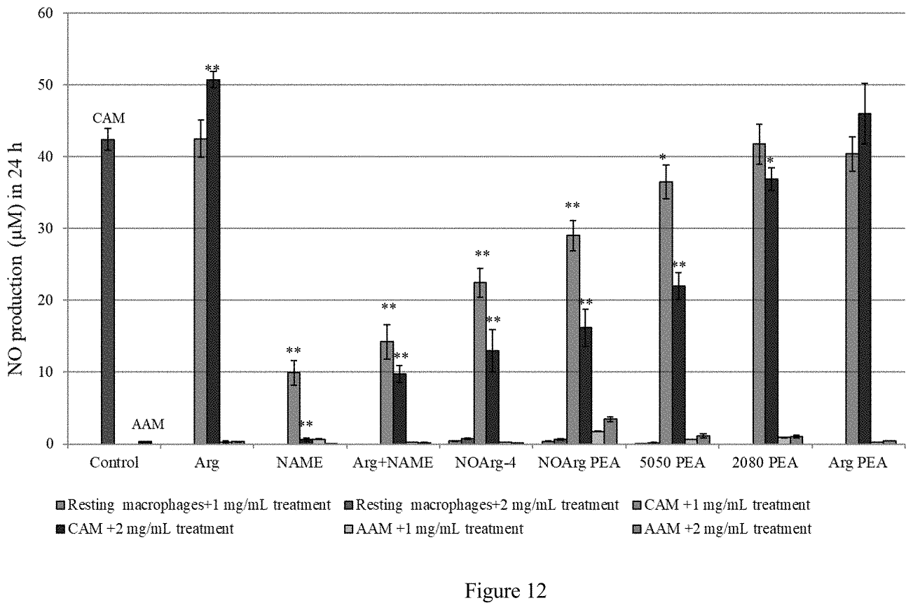

As shown in FIG. 12A, CAM produced a much higher NO level when compared to the resting macrophages or AAM. Increasing extracellular Arg concentration elevated the NO production of CAM, i.e., 50.7 1.1 μM (2 mg/mL Arg)>42.5±2.6 μM (1 mg/mL Arg) 42.4 1.5 μM (CAM control). It is the so-called Arg paradox: the dependence of cellular NO production on exogenous Arg concentration despite the theoretical saturation of NOS with intracellular Arg.

Among all testing samples, L-NAME suppressed the NO production of CAM, 9.9±1.7 μM NO at 1 mg/mL and 0.6±0.2 μM NO at 2 mg/mL. The effective concentration of L-NAME ranged from 0.1 mmol/L (27 μg/mL) to 10 mmol/L (2.7 mg/mL) when treating cells and tissues. NOArg-4 monomer is a less potent inhibitor compared to L-NAME (22.4±2.0 μM NO from NOArg-4 monomer vs. 9.9±1.7 from L-NAME at 1 mg/mL, and 13.0±2.9 μM NO from NOArg-4 monomer vs. 0.6±0.2 from L-NAME at 2 mg/mL), probably because L-NAME can efficiently compete with Arg to bind to the oxygenase domains of iNOS, whereas NOArg-4 is a larger molecule with two nitroguanidine groups which are not always bound to iNOS simultaneously. The inhibitory effect of NOArg-Arg co-PEAs is less potent than the NOArg-PEA. The NO level of CAM treated by Arg-PEA showed no statistically significant difference compared to the CAM control. By adjusting dosage and composition of NOArg-Arg co-PEA in the treatments, the concentration of NO produced by CAM could be controlled.

In FIG. 12B, the CAM incubated with PEA treatments showed significantly higher arginase activities than the CAM control. A higher NOArg composition in the NOArg-Arg co-PEA led to a higher arginase activity in CAM, i.e., NOArg-PEA (30.5 U)>50/50-PEA (29.0 U)>20/80-PEA (25.0 U)>CAM control (16.5 U) at 2 mg/mL. Arginase activity of both the Arg and 2-Arg-4 PEA treatments (up to 2 mg/mL) showed no significant difference compared to the CAM control. In the resting macrophage condition, extracellular Arg concentration, L-NAME treatment, and PEA treatments have no significant effect on the arginase activity compared to the control.

There are two isoforms of arginase, arginase I (cytosolic) and arginase II (mitochondrial). AAM dominantly express arginase I, while arginase II is associated with pro-inflammatory responses. Murine and human CAM upregulates iNOS and arginase II, but not arginase I. The possible explanation to the arginase activity change after the variety of treatments is because N-co-hydroxy-L-arginine, which is an intermediate in the NOS pathway and the nitrite which is the oxidized product of NO, can inhibit arginase activity. It has been reported that intermediate products of the NOS pathway (citrulline, nitrite, and the like) were shown to be able to inhibit the arginase activity of macrophages and a NOS inhibitor (N-methylarginine) enhanced arginase activity of macrophages. In this in vitro test, the nitrite and N-o-hydroxy-L-arginine produced by CAM control and Arg-PEA treated CAM without iNOS inhibition might lower the arginase activity compared to NOArg-PEA and NOArg-Arg co-PEAs in FIG. 12B.

Arginase I is dominantly expressed in the AAM. NOArg-4 (36.7±0.9 U), NOArg-4 PEA (36.7±1.9 U) and 20/80-PEA (34.4±1.3 U) showed a statistically higher arginase activity than the AAM control (28.4±2.3 U) at 2 mg/mL. Small molecular weight NOS inhibitors led to a significantly higher arginase activity in AAM, i.e., 2 mg/mL L-NAME (35.8±1.0 U), 2 mg/mL NOArg-4 (36.7±0.9 U). A possible explanation is that the arginase I gene expression is inducible in macrophages by a variety of stimuli besides IL-4, for example, by elevated cAMP, GM-CSF, IL-10 and TGF-β. Small molecule NOS inhibitor treatment might influence the level of these factors which can upregulate the expression of arginase I.

After the acute inflammatory stage of wound healing, NO production is suppressed, resulting in an increase in arginase activity from macrophages. The arginase from macrophage may also participate in the fibrogenic process via the synthesis of Ornithine-derived proline and anti-inflammatory action via the production of polyamines. An elevated arginase activity of macrophages in the late inflammatory stage may also decrease the NO level by competing for the Arg substrate for arginase activity. Notably, the arginase activity of fibroblasts could also be induced by IL-4 and TGF-β1.

TNF-α and TGF-β1 production of RAW 264.7 cells with treatments.

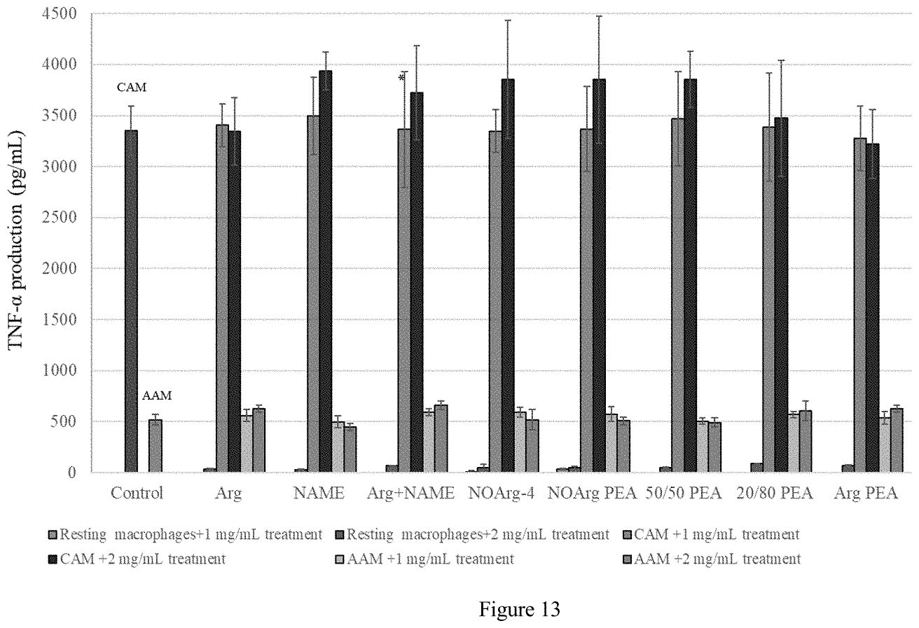

CAM is characterized by the production of proinflammatory cytokines like TNF-α, IL-1, and IL-6 in the early stage of the inflammatory response in wound repair, whereas AAM produces fewer TNF-α and elevated arginase I in the later stage of wound repair. TNF-α production from the 3 phenotypes of macrophages (resting, CAM and AAM) after treated with the PEAs is shown in FIG. 13A. As expected, no TNF-α production was detected from the resting macrophages control. With NOArg-PEA, 50/50-PEA or 20/80-PEA treatments, resting macrophages showed very low levels of TNF-α production (0-88.2 μg/mL). The CAM control produced more than 6 folds of TNF-α (3,349.4±243.3 μg/mL) when compared to the AAM control (518.3±52.5 μg/mL). 2 mg/mL treatments of Arg+L-NAME, NOArg-4 monomer, NOArg-PEA, 50/50-PEA, and 20/80-PEA resulted in TNF-α production of 3,722.2±461.6, 3,855.8±579.9, 3,851.7±625.9, 3,856.1±277.1, and 3,474.5±569.1 μg/mL, respectively. Only the 2 mg/mL L-NAME treatment (3,937.0±183.9 μg/mL) produced a significantly higher TNF-α than the CAM control. No significant change of TNF-α level from all the treatments in the AAM condition was found when compared with the AAM control. Although iNOS and TNF-α are two independent pathways, some studies reported NOS inhibitors could cause a concentration-dependent increase of TNF-α release from CAM in vitro and in vivo, which may explain the increased TNF-α in CAM treated with L-NAME. Meanwhile, the production of TNF-α from CAM was barely affected by the treatments of either NOArg-PEA or NOArg-Arg co-PEA probably because the inhibitory effect of PEA treatments is much lower than the small molecule inhibitors (i.e., 2 mg/mL L-NAME) (FIG. 12A, FIG. 13A).

TGF-β1 is a potent anti-inflammatory mediator in the wound healing process. In FIG. 13B, resting RAW 264.7 cells produced a low level of TGF-β1 (20.4±10.3 μg/mL) and all treatments did not significantly vary the TGF-β1 level. On the contrary, the CAM treated with NOArg-PEA and NOArg-Arg co-PEAs showed a significantly higher dose-dependent TGF-β1 production (2-3 folds) than the untreated CAM control (71.6±16.4 μg/mL). For example, 1 mg/mL of NOArg-PEA, 50/50-PEA, 20/80-PEA treatments led to 137.2±6.4, 151.5±9.6, and 158.3±23.1 μg/mL TGF-β1 production, respectively. An increased dosage also led to an increase in TGF-β1 production, e.g., for NOArg-PEA, TGF-β1 production was increased to 225.5±7.2 μg/mL at 2 mg/mL. Whereas the TGF-β1 level of AAM treated with NOArg-PEA or NOArg-Arg co-PEAs showed no significant difference compared to the AAM control. Several studies reported inhibiting NO production were associated with an increased TGF-β1 expression in vitro and in vivo, while Arg supplement could have opposite effect due to the elevated NO biosynthesis. For NOS inhibitor-treated chondrocytes, the decreased NO resulted in an elevated level of TGF-β1 through relieving the suppression of IL-1β. Macrophages may have a similar mechanism. CAM with a variety of treatments produced different concentrations of NO, hence the TGF-β1 production differs. In contrast, AAM's NO level is too low to influence TGF-β1 expression (FIG. 12A).

The CAM treated with NOArg-PEA, 50/50-PEA and 20/80-PEA showed increased TGF-β1 production, which may regulate the phenotypes of multiple cells and facilitate the wound healing from pro-inflammatory stage to anti-inflammatory stages. TGF-β1 inhibits iNOS activity and increases arginase, ornithine decarboxylase and ornithine aminotransferase activity, therefore stimulates the CAM to AAM switch. TGF-β1 also increases the expression of ED-A fibronectin. In the presence of mechanical stress, TGF-β1 and ED-A promote the modulation of proto-myofibroblasts into differentiated myofibroblasts.

In this in vitro study, NOArg-PEA and NOArg-Arg co-PEA treated CAM have shown lower NO production, higher arginase activity, and higher TGF-β1, indicating their potential role in facilitating the transition of a pro-inflammatory phase to a pro-healing phase in wounds. The Arg-PEA and NOArg-Arg co-PEA treatment may also alter the levels of other metabolism products from macrophages, such as citrulline, ornithine, and polyamine etc. which also play important roles in the collagen synthesis and cell proliferation and are hard to be detected in the cell culture media in vitro. Due to the complex and dynamic nature of the wound microenvironment, the efficacy of the PEA material treatments was further tested in the following in vivo experiment.

Evaluation of wound healing in vivo and histological analysis.

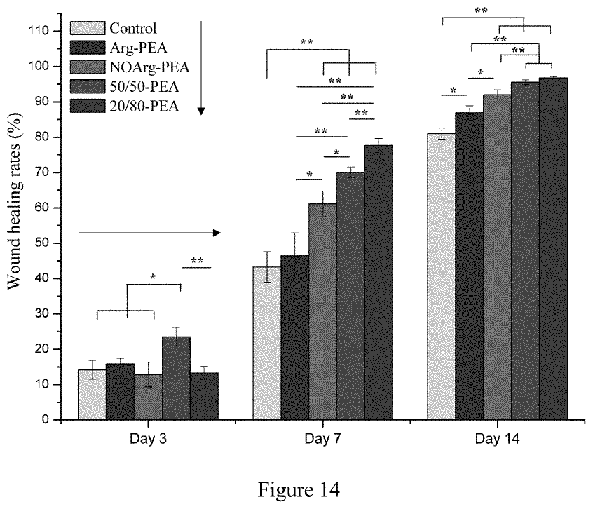

FIG. 14A shows the representative images of the full-thickness wound beds treated with various PEAs during the wound healing process of diabetic rat models over 14 days. The image data illustrate that all those wounds treated with NOArg-PEA, 50/50-PEA, and 20/80-PEA, achieved a significantly larger reduction in wound area when compared with the control. Such a wound area reduction becomes apparent as early as day 7, i.e., the wound healing rates were significantly accelerated by NOArg-PEA, and 50/50-PEA and 20/80-PEA. FIG. 14B shows the quantification of those corresponding wound area reduction image data upon different treatments. The 20/80-PEA group achieved the highest healing rate of 78%, followed by 50/50-PEA (70%), NOArg-PEA (62%), Arg-PEA (45%) and control (43%) on day 7. On day 14, the wounds in the 20/80-PEA and 50/50-PEA groups showed more than 95% healing rate, demonstrating that the design concept of combining both Arg and NOS inhibitor in polymeric biomaterials (i.e., 50/50-PEA or 20/80-PEA) significantly accelerated the wound healing process of diabetic rat models. 20/80-PEA treated animal group showed a significant faster wound area reduction than all other treatments on day 7 and a significantly faster healing than other treatments except 50/50-PEA treatment on day 14. This indicates the NOArg-Arg co-PEAs have more efficacy in accelerating wound closure.

The healing quality of the treated diabetic wounds was assessed by H&E staining of the wound site tissue. As shown in FIG. 8A, the wound beds were filled with fibrin clots in all groups on day 3. The fibrin clot serves as a temporary shield to protect the exposed tissues and provides a provisional matrix through which cells can migrate during the healing process. The fibrin clots also contain a variety of cytokines and growth factors, which can recruit circulating inflammatory cells and initiate the wound healing process. Neutrophils and macrophages started to infiltrate into the wound beds (dark region on the right in the images in FIG. 8). The resident dermal fibroblasts close to the wound begin to migrate into the provisional fibrin clot and lay down the collagen-rich matrix 3-4 days after wound insult. Table 3. Lists the severity of inflammatory cell infiltration in the wound tissue at different time points. On day 3, 50/50-PEA and 20/80-PEA treatment did not dampen the inflammatory cell infiltration in the wound tissue compared to control, while NOArg-PEA and Arg-PEA treated wound showed less inflammatory cells. On day 7, control and Arg-PEA treatment showed a severe or moderate inflammatory cell infiltration and all PEA treatments with NOArg composition showed a mild inflammatory cell infiltration. On day 14, the inflammatory cell was not detected by H&E staining in the 50/50-PEA treated wound and all other treatments and control showed mild levels of inflammatory cells. PEAs did not influence the inflammatory cell infiltration at early stage is probably because: (1) biodegradation requires 2-3 days, (2) PEAs with NOArg composition may decrease the concentration of metabolic products of iNOS pathway in the wound, so they have more effect on the continuously recruited inflammatory cells instead of the cells migrated to the wound at the early stage. On day 7 and day 14, NOArg-PEA, 20/80-PEA, particularly 50/50-PEA treatment showed an earlier resolution of inflammation stage and transformation towards the proliferative stage of the wound healing process than the control and Arg-PEA treatment. Meanwhile, keratinocytes started to migrate and formed a typical epidermal tongue at the wound edge in all groups (FIG. 8A). The 20/80-PEA and 50/50-PEA treated wounds showed a more completed formation of neo-epidermis than other treatments and control, i.e., faster re-epithelialization. The NOArg-PEA treated wound also showed a better re-epithelialization than the Arg-PEA treatment and control. In wound healing, re-epithelialization is the critical step for forming a physical barrier against external bacteria and other harmful substances. All wounds except the control were fully covered with neo-epidermis 14 days after surgery. The 20/80-PEA and 50/50-PEA treated wounds had thicker layers of neo-epidermis across the entire wound beds (FIG. 8B, black arrow).

TABLE 3
Severity of inflammatory cell infiltrate

| | Cell infiltrate | Cell infiltrate | Cell infiltrate |
|---|---|---|---|
| Treatment | (Day 3) | (Day 7) | (Day 14) |
| Control | 3 | 3 | 1 |
| NOArg-PEA | 2 | 1 | 1 |
| 50/50-PEA | 3 | 1 | 0 |
| 20/80-PEA | 3 | 1 | 1 |
| Arg-PEA | 2 | 2 | 1 |

0 = no inflammatory cell infiltrate; 1 = mild inflammatory cell infiltrate; 2 = moderate inflammatory cell infiltrate; 3 = severe inflammatory cell infiltrate.

Immunohistochemical Analysis.

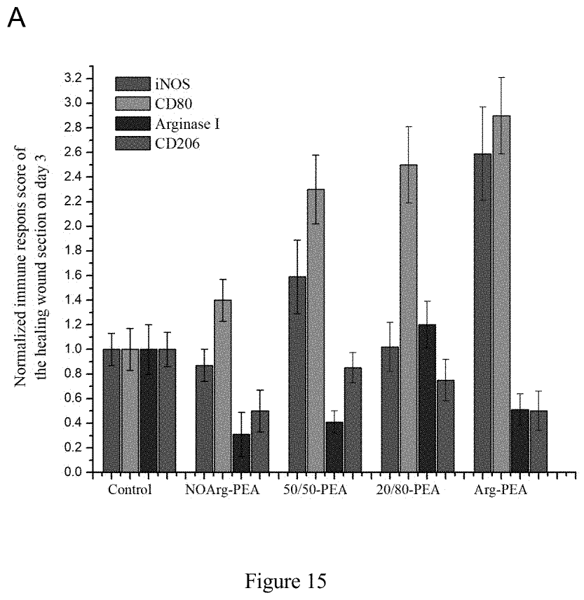

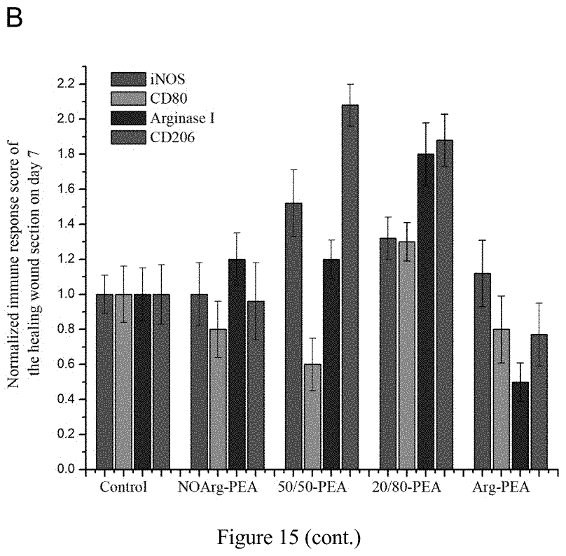

The immunohistochemical analysis of the wound tissue microenvironment (mainly the proinflammatory/prohealing phenotypes of infiltrated macrophages) on day 3 and day 7 is summarized in FIG. 15 and Table 5. In a wound healing process, macrophages mainly appear in inflammation stage and granulation formation stage (approximately day 3-day 10). The analysis of tissue sections on day 14 is not given, because the number of macrophages rapidly decrease via apoptosis in the wound tissue after day 11. As shown in FIGS. 15A and C, on day 3, 50/50-PEA, 20/80-PEA and Arg-PEA treated wound showed a similar or higher iNOS level, only NOArg-PEA treated wound showed a slightly lower iNOS expression level. CD80 marker was used to show the proinflammatory cell density (may including neutrophil, monocyte, monocyte-derived macrophages on day 3, mainly CAMs on day 7) in wound. All wounds treated with PEAs showed higher CD80 level that indicates more cells with inflammatory phenotype infiltrated to the wound bed than the untreated control. And Arg-PEA treatment showed both the highest iNOS expression and CD80 on day 3. NOArg-PEA and NOArg-Arg co-PEA treatments showed higher CD80 expression compared to control (FIG. 15C, Table 5) indicates the NOArg-PEA and NOArg-Arg co-PEA which contain iNOS inhibitory component did not hamper the recruitment of inflammatory cells at the early stage (day 3). This is consistent with the histology data of FIG. 7 and Table 3. Compared to the control, Arg-PEA induced the highest inflammatory response (both iNOS and CD80) on day 3 because the topical Arg supplement to a diabetic wound may aggravate inflammation due to a higher concentration of the metabolic product of iNOS pathway. On day 3, all PEA treated wound also showed less CD206 (mannose receptor, a biomarker for AAM) compared to the control (FIG. 15B) and most PEA treatments also showed lower arginase I expression except 20/80-PEA treatment led to a slightly higher arginase I level than the control. Arginase I and CD206 are the biomarkers of AAMs phenotype which take part in the dampening of inflammation, the promotion of tissue remodeling, angiogenesis, and immunoregulation. The normalized CD206/CD80 ratio in Table 4 indicates the overall cell phenotype in all PEA treated wounds is more pro-inflammatory than the control and Arg-PEA treated wound showed the highest pro-inflammatory response (highest iNOS expression and highest CD80 positive cells).

On day 7, all the PEA-treated wounds and control showed decreased iNOS level and increased arginase-1 level compared to day 3 (FIG. 15 A, B, D, and Table 5). When compared to the control, NOArg-PEA treated wound showed a slightly more anti-inflammatory environment (less CD80 positive cells, higher arginase I, similar iNOS and CD206). The 50/50-PEA treated wound showed the most anti-inflammatory microenvironment (low iNOS/CD80 and high arginase I/CD206). The 20/80-PEA showed slightly higher iNOS/CD80 expression and a much higher arginase I/CD206 expression. Arg-PEA treated wound also showed a slightly reduced inflammation (similar iNOS expression as control, less CD80 positive cells) but also less pro-healing features (lower arginase I and CD206) on day 7. In Table 4, a representative normalized CD206/CD80 ratio data shows the NOArg-Arg co-PEAs and NOArg-PEA treatments led to a higher AAM phenotype ratio in the wound than the control and Arg-PEA treatment. In sum, the 50/50-PEA and 20/80-PEA treated wounds showed more pro-healing features on day 7 than the NOArg-PEA treated, Arg-PEA treated or control. This in vivo wound healing data illustrates: neither Arg-PEA nor NOArg-PEA can provide a near optimal and timely transformation of a wound from its acute inflammatory to pro-healing stages. A proper balance of Arg and NOS inhibitor composition is required for achieving a better healing of diabetic wounds (FIG. 14).

TABLE 4
Normalized CD206/CD80 IR score ratio (AAM/CAM) in the wound tissue

| Normalized | | | | | |
|---|---|---|---|---|---|
| CD206/CD80 | | | | | |
| IR score ratio | | | | | |
| in the wound | | | | | |
| section | Control | NOArg-PEA | 50/50-PEA | 20/80-PEA | Arg-PEA |
| Day 3 | 1.00 | 0.36 | 0.37 | 0.30 | 0.17 |
| Day 7 | 1.00 | 1.20 | 3.47 | 1.45 | 0.96 |

*Larger ratio indicates a more anti-inflammatory cell phenotype

Collagen Deposition Evaluation.

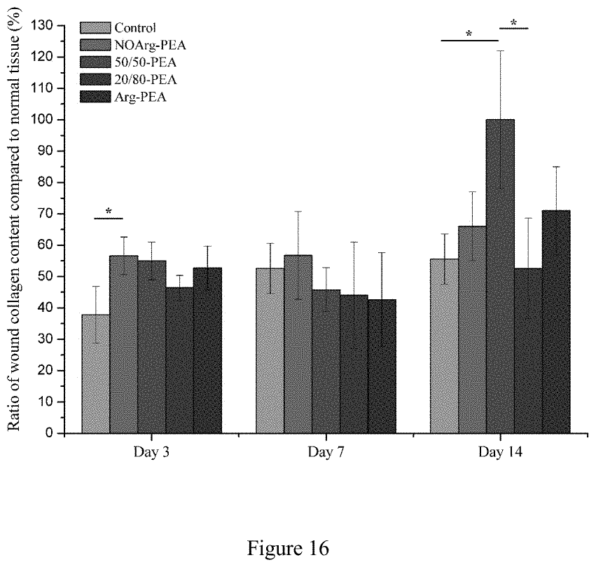

The levels of newly synthesized collagen in the regenerated tissues upon the treatments by the PEA biomaterials are shown in FIG. 16. On days 3 and 7, a limited deposition of collagen was observed in the wound beds of all groups (blue fibrous structure on the right, FIG. 16A) as the wounds were still in the inflammatory stage. NOArg-Arg co-PEA or Arg-PEA treatment did not show an increased collagen content in the neo-tissue. Only NOArg-PEA treatments showed slightly more collagen content than the control on day 3. Most collagen synthesis happens in the granulation tissue formation. On day 14, 50/50-PEA treated wound showed the highest level of newly synthesized collagen (100% compared to the unwounded tissue) with a densely packed fibrous structure. Arg-PEA also showed higher collagen contents (71.0%) than the NOArg-PEA (66.0%) and 20/80-PEA (62.6%) treated wound beds and control groups (55.6%) (FIG. 16B). Even though without NOS inhibition effect, Arg-PEA treated wound showed faster healing rate (FIG. 14B) and higher collagen content compared to control. This beneficial effect of Arg composition in the treatment on healing was shown in animal studies but was not evident from the in vitro data is because: (1) macrophages in vitro were cultured with sufficient L-Arg in the complete media. Additional Arg-PEA supplement could not differ from the untreated control. Whereas in the wound healing environment, the Arg concentration is usually decreased to a low level because of the consumption; (2) Arg supplement in vivo probably increased ornithine (one precursor of collagen), citrulline, polyamines (promoting cell proliferation) production which are beneficial to healing. The in vitro experiment did not include the assays of these metabolism products.

Angiogenesis in Wounds.

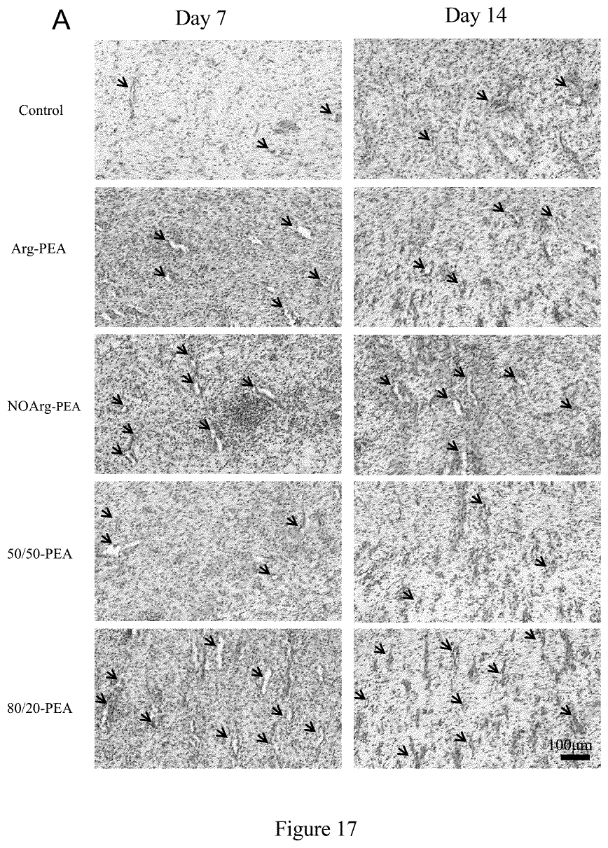

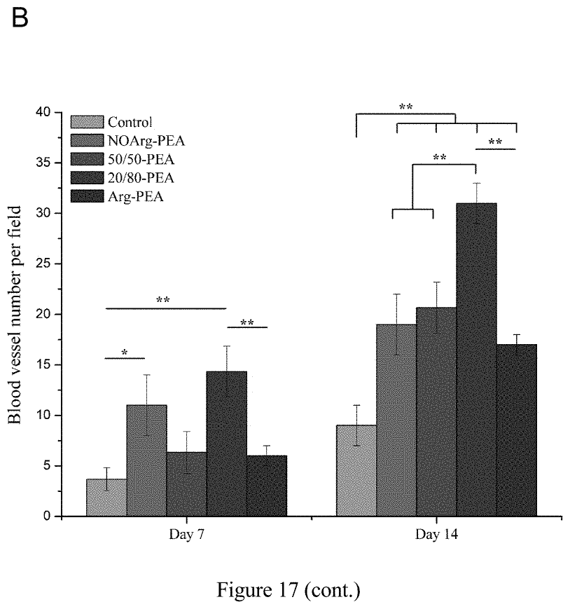

The neovascularization of the wound sites was detected by the CD31 immunohistochemical staining on day 7 and day 14. FIG. 17 exhibited that all PEA biomaterials significantly stimulated the formation of new vessels in wounds compared with the control on day 14; the largest number of microvessels being observed in wounds treated with 20/80-PEA. On day 7, only NOArg-PEA and 20/80-PEA treated wounds showed significantly more microvessels in comparison with the control. It is well recognized that NO produced by iNOS has angiogenic effects. Hence, the supplement of Arg, which increased the NO production, was supposed to lead to an enhanced angiogenesis. Compared to 50/50-PEA, 20/80-PEA promoted more new vessels formation. Notably, Arg-PEA treatment, which has the highest percentage of Arg did not result in the best angiogenesis. A possible reason is Arg-PEA treatment may only induce NO production without promoting the pro-healing cell phenotypes in the wounds (FIG. 15) which usually upregulate the arginase metabolism pathway. Besides NO, the metabolic products of the arginase pathway, like polyamine, can stimulate angiogenesis by promote the proliferation of endothelial cells, which may explain the better angiogenesis in all PEA biomaterials.

In summary, NOArg-Arg co-PEA treatments, i.e. 20/80-PEA and 50/50-PEA showed faster wound healing rate (FIG. 14), faster and better re-epithelialization (FIG. 15) and lower inflammatory cell infiltration in late inflammatory stage and thereafter (day 7 and day 14, Table 3), higher M2/M1 cell phenotype ratio on day 7 (Table 4), more collagen content/angiogenesis in the wound on day 14 (FIG. 16 and FIG. 17) than the Arg-PEA, NOArg-PEA and untreated control that may make them promising formulations for the impaired wound healing treatment.

Conclusions.

A new family of water-soluble and biodegradable NOArg-PEA and NOArg-Arg co-PEAs were designed and synthesized for the purpose of modulating the wound microenvironment for a faster and better healing, particularly for those challenging wound healing conditions like diabetic wounds. These polymeric biomaterials have Arg and NOS inhibitor (NOArg) building blocks at predetermined composition ratios and were found noncytotoxic toward murine macrophages and fibroblasts. The chemistry of these biomaterials can be tuned for achieving different levels of NO, cytokine, and growth factors production from CAM or AAM in vitro. They reduced the NO production from the CAM to various extents, increased the arginase activity in both CAM/AAM, increased TGF-β1 production of CAM, and had no significant effect on TNF-α production. The diabetic rats with full-thickness incisional wounds treated with PEAs and NOArg-Arg co-PEAs showed faster-wound healing rate than the control on days 7 and 14. The NOArg-Arg co-PEA treatments led to a more pro-healing wound microenvironment and improved re-epithelialization rate around day 7. They also increased the collagen deposition and angiogenesis in the wound beds on day 14. This new approach may provide a viable alternative to advance the treatment of impaired wound healing, like diabetic wounds.

TABLE 5
The immunoreactivity (IR) score of immunohistochemical
staining of PEAs treated wound tissue

| | | % | Grade* | Intensity | | IR1: |
|---|---|---|---|---|---|---|
| | | Positive | Positive | of Positive | IR0 | Normalized |
| | Samples | Pixels | Pixels | Pixels | Score^{#} | to Control |
| iNOS | | | | | | |
| Day 3-5 | Control | 71 | 3 | 1.52 | 4.6 | 1.00 |
| Day 3-2 | NOArg-PEA | 65 | 3 | 1.33 | 4.0 | 0.87 |
| Day 3-3 | 50/50-PEA | 80 | 4 | 1.82 | 7.3 | 1.59 |
| Day 3-4 | 20/80-PEA | 73 | 3 | 1.57 | 4.7 | 1.02 |
| Day 3-1 | Arg-PEA | 100 | 4 | 2.98 | 11.9 | 2.59 |
| Day 7-5 | Control | 22 | 2 | 1.25 | 2.5 | 1.00 |
| Day 7-2 | NOArg-PEA | 24 | 2 | 1.25 | 2.5 | 1.00 |
| Day 7-3 | 50/50-PEA | 99 | 4 | 2.35 | 9.4 | 3.76 |
| Day 7-4 | 20/80-PEA | 58 | 2 | 1.65 | 3.3 | 1.32 |
| Day 7-1 | Arg-PEA | 34 | 2 | 1.40 | 2.8 | 1.12 |
| CD80 | | | | | | |
| Day 3-5 | Control | 19 | 2 | 0.40 | 0.8 | 1.00 |
| Day 3-2 | NOArg-PEA | 25 | 2 | 0.55 | 1.1 | 1.40 |
| Day 3-3 | 50/50-PEA | 41 | 2 | 0.90 | 1.8 | 2.30 |
| Day 3-4 | 20/80-PEA | 46 | 2 | 1.00 | 2.0 | 2.50 |
| Day 3-1 | Arg-PEA | 51 | 2 | 1.15 | 2.3 | 2.90 |
| Day 7-5 | Control | 28 | 2 | 0.60 | 1.2 | 1.00 |
| Day 7-2 | NOArg-PEA | 23 | 2 | 0.50 | 1.0 | 0.80 |
| Day 7-3 | 50/50-PEA | 15 | 2 | 0.30 | 0.6 | 0.60 |
| Day 7-4 | 20/80-PEA | 19 | 2 | 0.40 | 0.8 | 1.30 |
| Day 7-1 | Arg-PEA | 16 | 2 | 0.30 | 0.6 | 0.80 |
| Arginase I | | | | | | |
| Day 3-5 | Control | 63 | 3 | 1.30 | 3.9 | 1.00 |
| Day 3-2 | NOArg-PEA | 29 | 2 | 0.60 | 1.2 | 0.31 |
| Day 3-3 | 50/50-PEA | 39 | 2 | 0.80 | 1.6 | 0.41 |
| Day 3-4 | 20/80-PEA | 75 | 3 | 1.56 | 4.7 | 1.20 |
| Day 3-1 | Arg-PEA | 94 | 4 | 0.50 | 2.0 | 0.51 |
| Day 7-5 | Control | 63 | 3 | 1.30 | 3.9 | 1.0 |
| Day 7-2 | NOArg-PEA | 75 | 3 | 1.53 | 4.6 | 1.2 |
| Day 7-3 | 50/50-PEA | 73 | 3 | 1.50 | 4.5 | 1.2 |
| Day 7-4 | 20/80-PEA | 79 | 3 | 2.33 | 7.0 | 1.8 |
| Day 7-1 | Arg-PEA | 47 | 2 | 1.00 | 2.0 | 0.5 |
| CD206 | | | | | | |
| Day 3-5 | Control | 43 | 2 | 1.00 | 2.0 | 1.00 |
| Day 3-2 | NOArg-PEA | 21 | 2 | 0.50 | 1.0 | 0.50 |
| Day 3-3 | 50/50-PEA | 37 | 2 | 0.85 | 1.7 | 0.85 |
| Day 3-4 | 20/80-PEA | 33 | 2 | 0.75 | 1.5 | 0.75 |
| Day 3-1 | Arg-PEA | 22 | 2 | 0.50 | 1.0 | 0.50 |
| Day 7-5 | Control | 27 | 2 | 1.30 | 2.6 | 1.00 |
| Day 7-2 | NOArg-PEA | 24 | 2 | 1.25 | 2.5 | 0.96 |
| Day 7-3 | 50/50-PEA | 65 | 3 | 1.80 | 5.4 | 2.08 |
| Day 7-4 | 20/80-PEA | 58 | 3 | 1.63 | 4.9 | 1.88 |
| Day 7-1 | Arg-PEA | 47 | 2 | 1.00 | 2.0 | 0.77 |

The percentage of positive pixels was graded as follows: Negative=0; ≤10%=1; 11≤50%=2, 51-80%=3; ≥81%=4.

An intensity score was assigned to strong positive pixels, positive pixels and weak positive pixels using the following formula:

$$Score = \frac{( Number of pixels in a zone ) \times ( Score of the zone )}{Total number of pixels in the image}$$

The score of the zone was 4 for strong positive pixels, 3 for positive pixels and 2 for weak positive pixels.

### Example 3

This example provides a description of nanoparticle formulation of polymers of the present disclosure.

There are 2 basic approaches to fabricate TIR-based AA-PEA nanoparticles (NPs): (1) adjust the x and y of the methylene groups in the Arg and NOArg-based diester monomers. (2) Incorporating a hydrophobic amino acid based diester as the 3^{rd} monomer in the copolymer structure.

Preparation of Arg/ArgNO PEA Based Nanoparticles Based on the (1) Approach Above (x and y Approach).

In this example, a series of L-nitroarginine (NOArg) and L-arginine (Arg) based PEA copolymers were prepared by the solution polycondensation, and the nanoparticles of the resulting copolymers were formulated via the self-assemble manner.

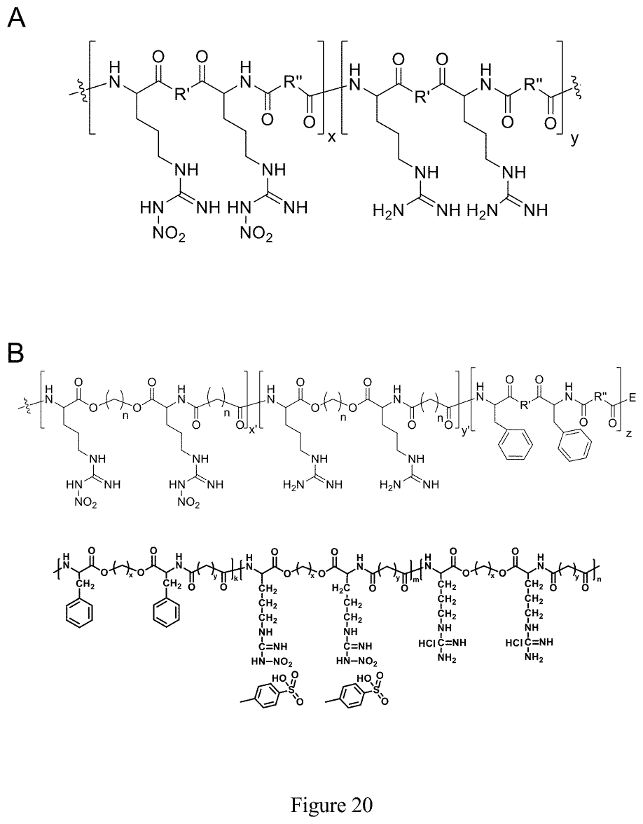

Synthesis of the Arg/NOArg-PEA copolymers: Arg/NOArg-4 and Arg-4-Cl diester monomers with different feeding ratios were copolymerized with di-p-Nitrophenyl Adipate (NA) or di-p-Nitrophenyl Sebacate (NS) monomers. The copolymer compositions are listed in Table 6. An example of the synthesis of the NOArg-Arg co-PEAs is given below to illustrate the details of the synthesis procedures. NOArg-4 (0.75 mmol), Arg-4-Cl (0.75 mmol) and NS (1.5 mmol) were dissolved in 10 mL of dry DMA, and excess triethylamine was added into the solution. The mixtures were stirred at 70° C. for 48 h. The resulting solution was precipitated with cold ethyl acetate, filtered and vacuum-dried. FIG. 20A shows the chemical structure of the NOArg-Arg PEA copolymer repeating unit. x and y can range from 1 to 12.

Formulation into nanoparticles: The synthesized copolymers were dissolved in DMSO with the concentration of 5 mg mL⁻¹ and dialyzed against DI water for 24 h. The copolymer solution after dialysis was collected. The size of self-assembled particles was measured using Malvern Zetasizer Nano and the morphology was observed by Transition Electron Microscope (TEM, FEI T12 Spirit TEM STEM).

Preparation of Phe/Arg/ArgNP Based Nanoparticles Based on the 2^{nd} Approach Listed Above (Adding a Hydrophobic Amino Acid Diester as the 3^{rd} Monomer).

The synthesis and self-assembling methods of NOArg-Arg-Phe co-PEAs were similar to the methods of NOArg-Arg co-PEAs. The copolymers compositions of this 2^{nd} approach are listed in Table 7. FIG. 20B shows the chemical structure of the NOArg-Arg-Phe PEA copolymer repeating unit.

Other hydrophobic amino acids: Leu, Gly, Ala, Val, Ile, Pro, Met, Trp.

TABLE 6
TIR-based AA-PEA Copolymer Composition
based on x and y Approach.

| | Monomer | NOArg-4/Arg-4-Cl | Zeta average | |
|---|---|---|---|---|
| Copolymer | combination | feeding ratio | size (nm) | PDI |
| P1 | NOArg-4 | 2/8 | / | / |
| P2 | Arg-4-Cl | 5/5 | 963 | 0.735 |
| P3 | NA | 8/2 | 721 | 0.62 |
| P4 | NOArg-4 | 2/8 | 366 | 0.143 |
| P5 | Arg-4-Cl | 5/5 | 651.2 | 0.148 |
| P6 | NS | 8/2 | 594.4 | 0.578 |

TABLE 7
TIR-based NOArg-Arg-Phe PEA Copolymer Composition based
on Hydrophobic Amino Acid Diester as the 3^{rd} monomer.

| | Monomer | NOArg-4/Arg-4-Cl | Zeta average | |
|---|---|---|---|---|
| Copolymer | combination | feeding ratio | size (nm) | PDI |
| P7 | Phe-4 | 2/2/8 | 252.2 | 0.242 |
| P8 | NOArg-4 | 2/5/5 | 544.4 | 0.199 |
| P9 | Arg-4-Cl | 2/8/2 | 688.3 | 0.595 |
| P10 | NA | 3/0/7 | 182 | 0.13 |
| P11 | | 3/4/3 | 564 | 0.098 |
| P12 | | 3/7/0 | 642 | 0.321 |

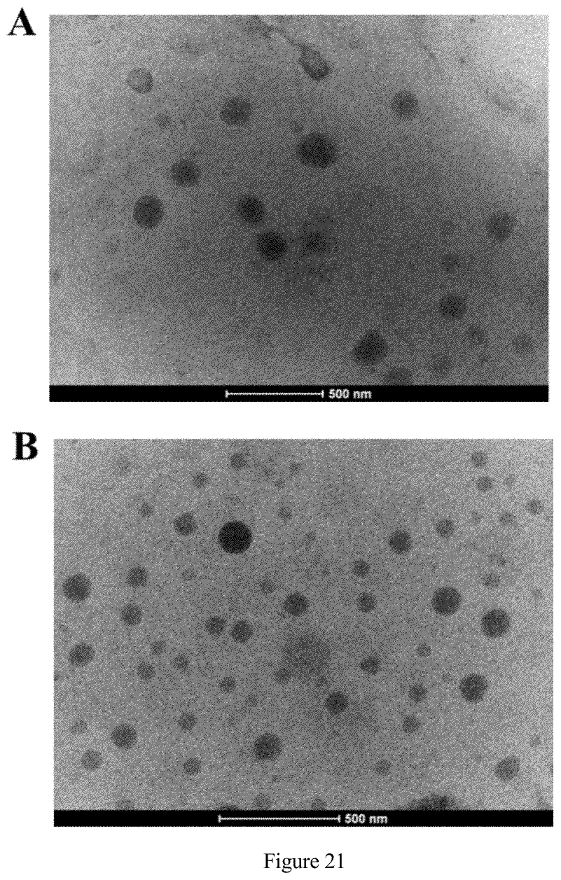

FIG. 21 shows TEM images of the NOArg-Arg and NOArg-Arg-Phe PEA copolymers self-assembled in aqueous solution, (A) P5 and (B) P8.

### Example 4

This example provides a description of polymers of the present disclosure.

TABLE 8

| | Healing | 20/80 PEA | 50/50 PEA |
|---|---|---|---|
| | Rate | 78% | 70% |
| NOArg-PEA | 62% | 125.8% | 112.9% |
| Arg-PEA | 45% | 173.3% | 155.6% |

Although the present disclosure has been described with respect to one or more particular embodiments and/or examples, it will be understood that other embodiments and/or examples of the present disclosure may be made without departing from the scope of the present disclosure.

1.-41. (canceled)

42. A method of treating a subject in need of treatment with one or more poly(ester amide) (PEA) polymer, comprising administering to the subject in need of treatment an effective amount of the one or more PEA polymer(s), wherein the PEA polymer comprises a first pendant group, wherein the first pendant group comprises an alkyl nitroguanidine, and at least a second pendant group chosen from an alkyl guanidine group and a side chain of a canonical amino acid, wherein the alkyl chain of the alkyl nitroguanidine group and alkyl guanidine group is 1 to 6 carbon atoms.

43. The method of claim 42, wherein the PEA polymer has the following structure: 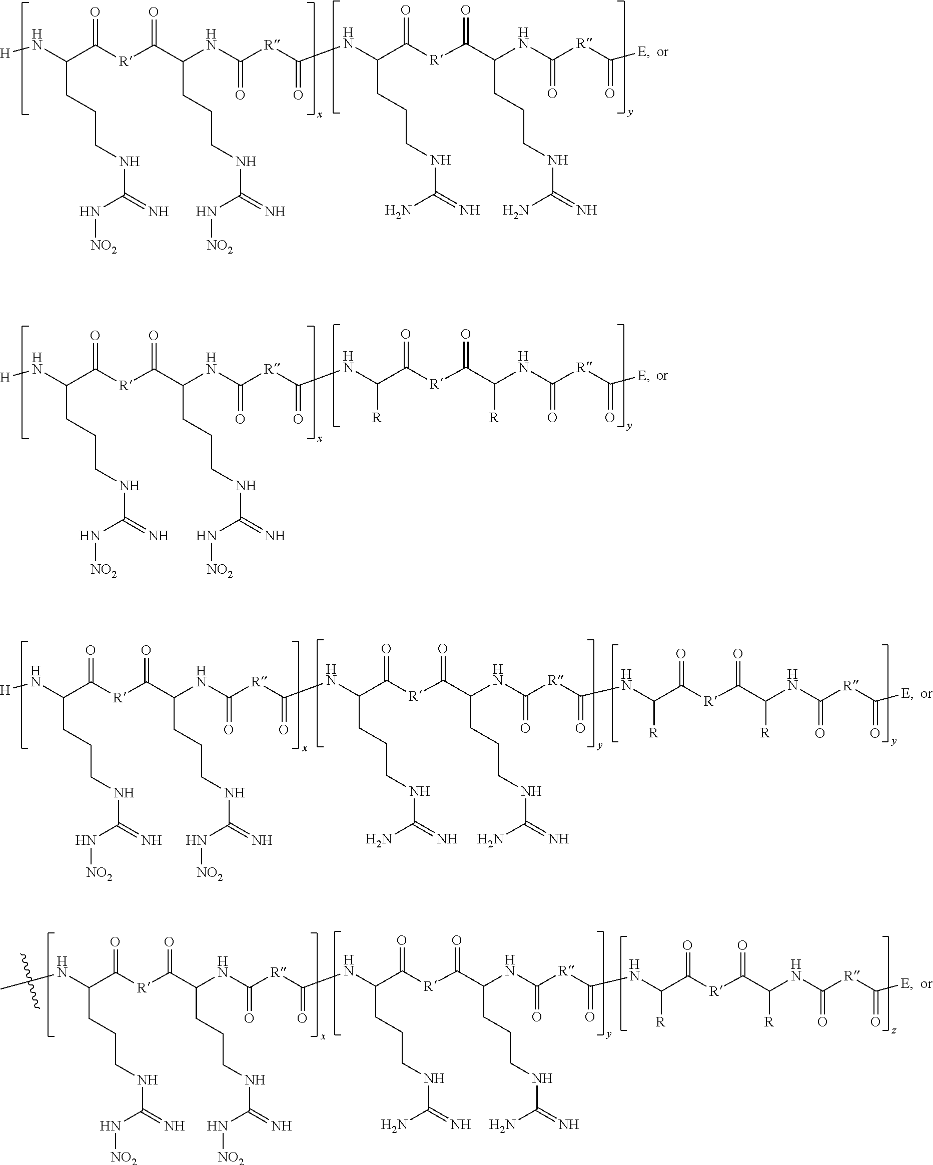 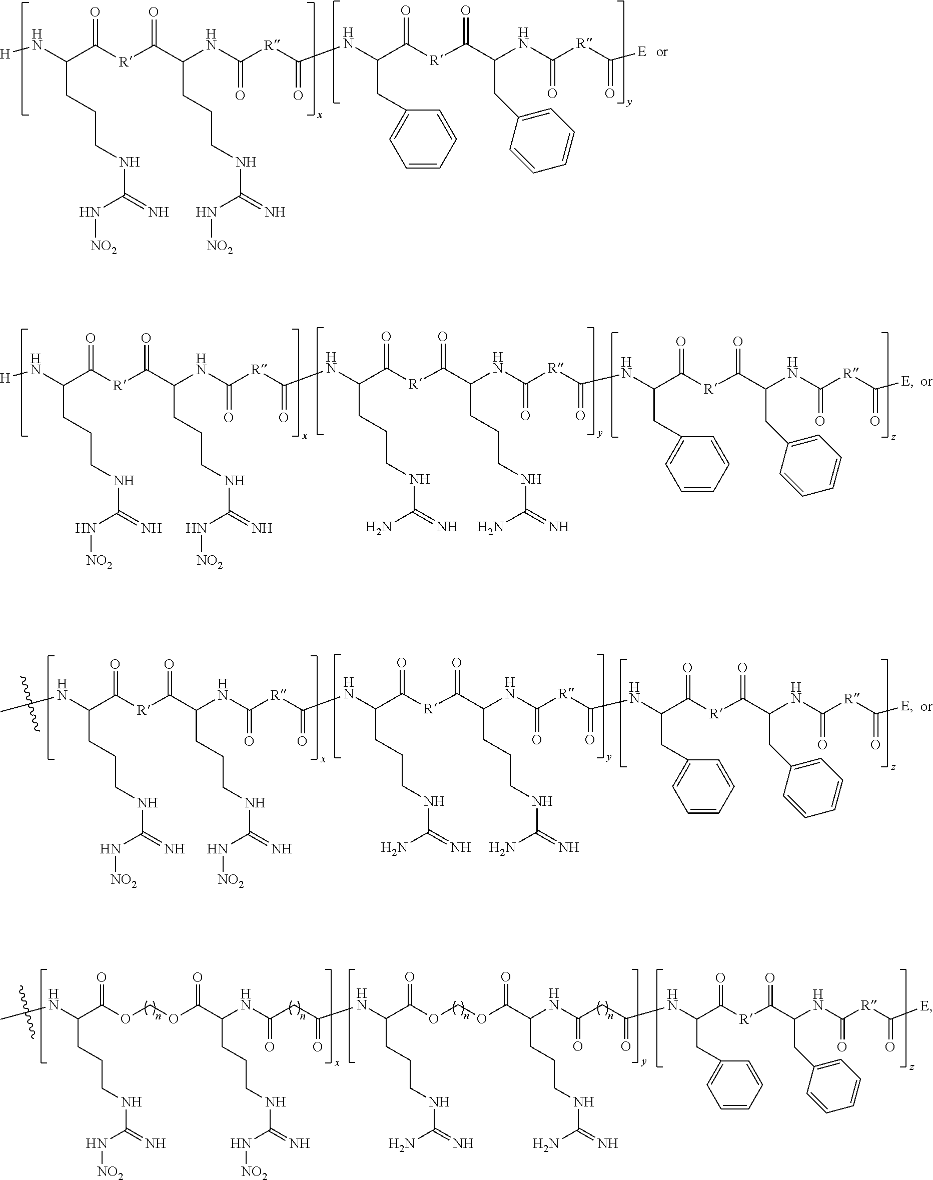
or a salt thereof,
wherein
R at each occurrence is independently chosen from the side chain of a hydrophobic amino acid and a cross-linking group;
R′ at each occurrence in the polymer is independently chosen from 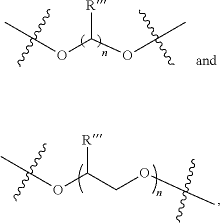
wherein R′″ at each occurrence in the polymer is independently chosen from H and an alkyl group, and wherein n is 1-10;
R″ at each occurrence in the polymer is independently chosen from 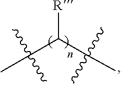
wherein R′″ at each occurrence in the polymer is independently chosen from H and an alkyl group, and n is 1-10;
E is chosen from 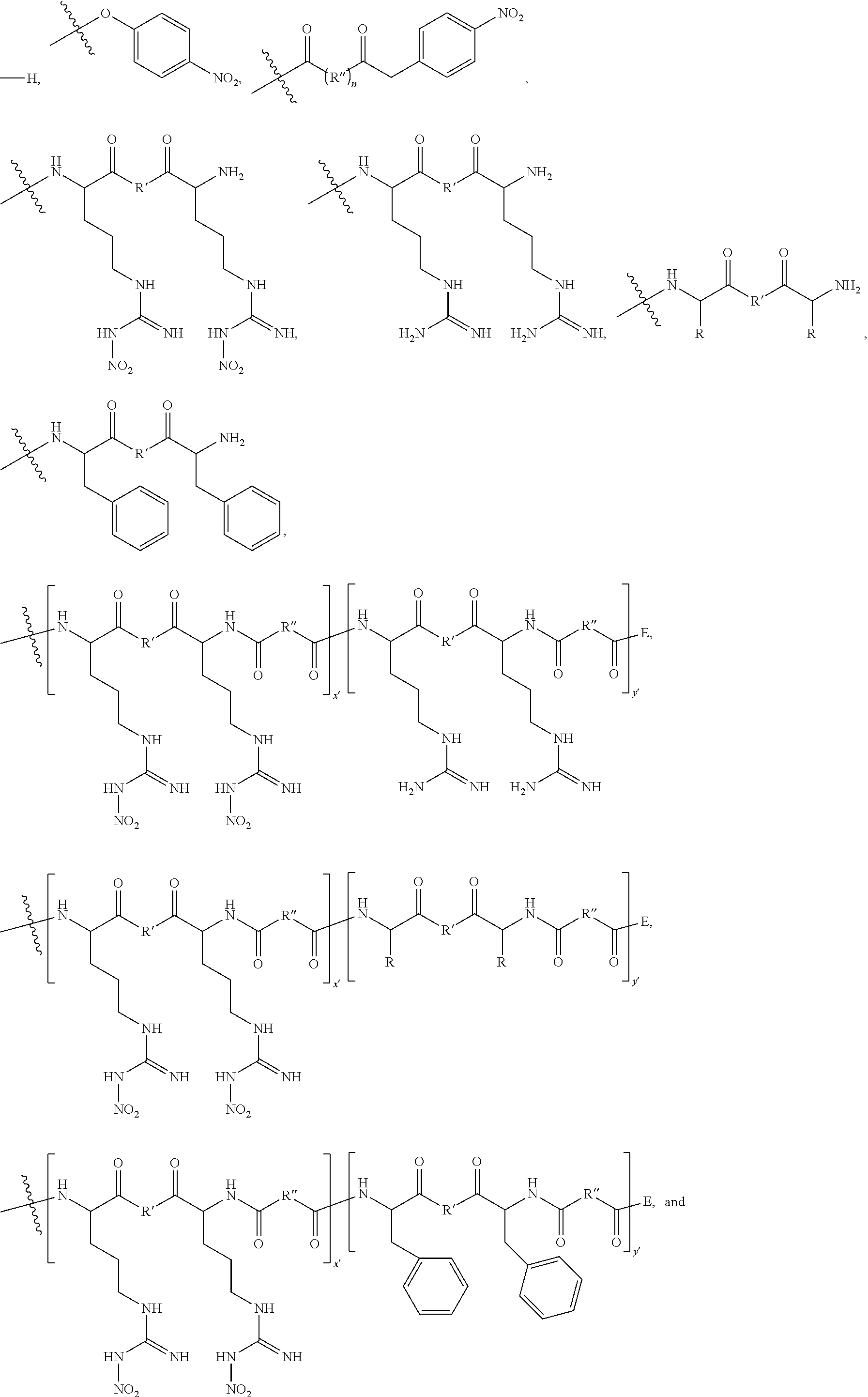 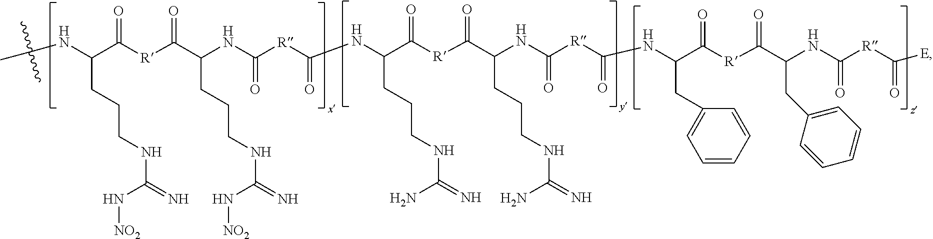
wherein R′ at each occurrence in the polymer is independently chosen from 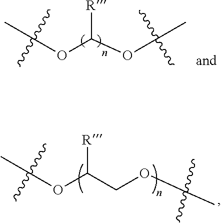
wherein R′″ at each occurrence in the polymer is independently chosen from H and an alkyl group, and wherein n is 1-10;
R″ at each occurrence in the polymer is independently chosen from 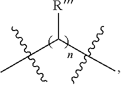
wherein R′″ at each occurrence in the polymer is independently chosen from H and an alkyl group, and n is 1-10;
x, y, z, x′, y′, and z′ are independently at each occurrence 0-100;
with the proviso: 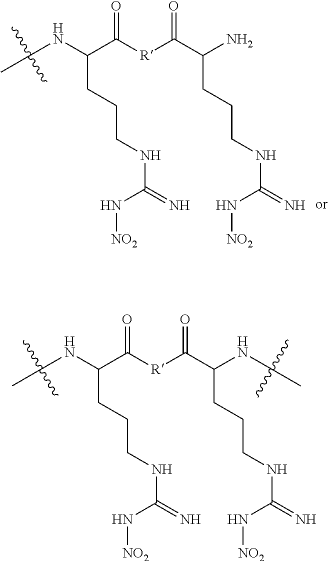
occurs at least once.

44. The method of claim 43, wherein the PEA polymer has the following structure: 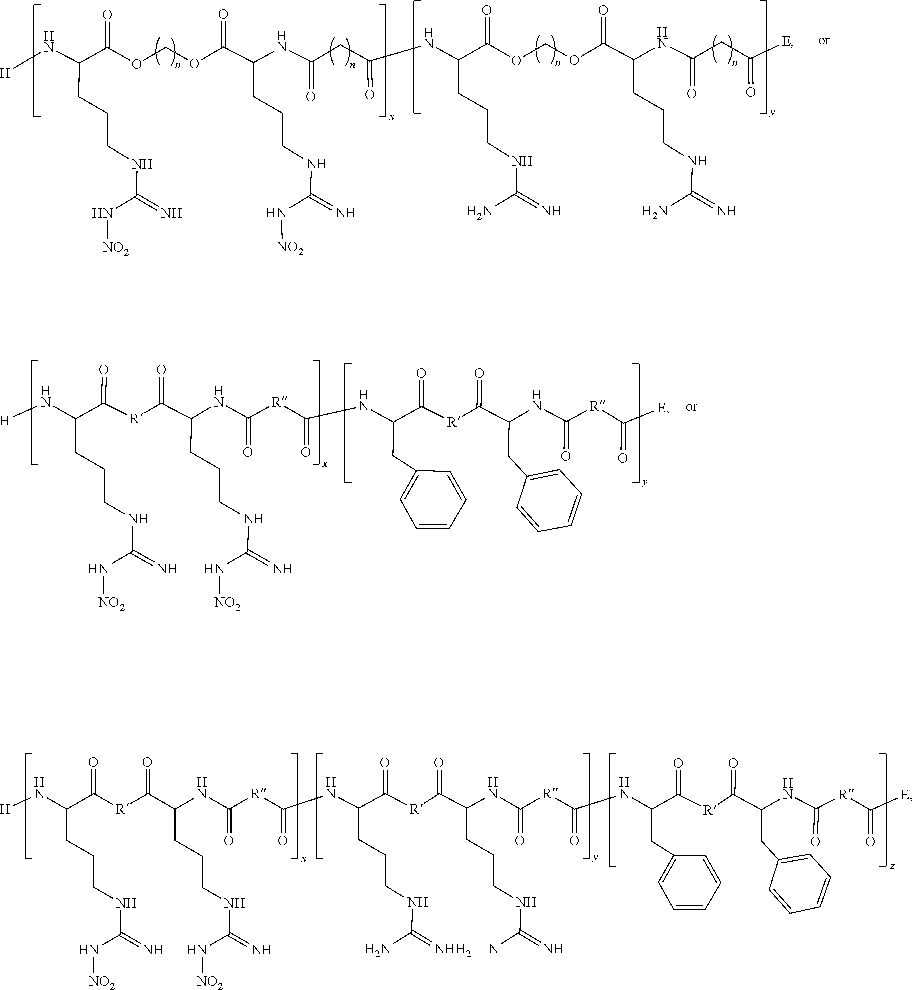
salt thereof, or a combination thereof, with the proviso that 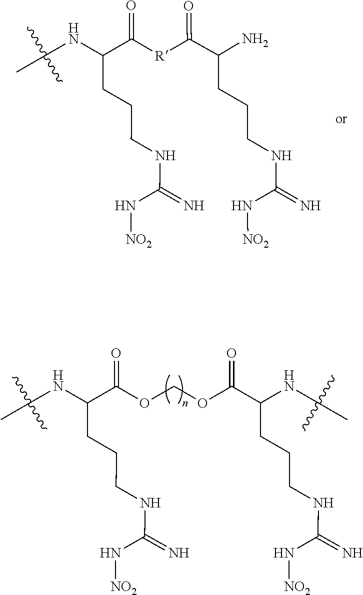
occurs at least once.

45. The method of claim 43, wherein the ratio of NOArg-4 to Arg-4 is 0.1-10:0-10.

46. The method of claim 45, wherein the ratio of NOArg-4 to Arg-4 is 1:1.

47. The method of claim 42, wherein the subject in need of treatment has one or more wound.

48. The method of claim 47, wherein the one or more wound is a diabetic wound, obesity associated wound, and/or pressure wound.

49. The method of claim 42, wherein the one or more wound is healed 40-81% faster than a wound treated with a 100% Arg-based PEA polymer.

50. The method of claim 49, wherein the one or more wound is healed at least 40% faster.

51. The method of claim 49, wherein the one or more wound is healed at least 70% faster.

52. The method of claim 42, wherein the one or more wound is healed 5-30% faster than one or more wound treated with a PEA polymer comprising a 100% NO-Arg-based PEA polymer.

53. The method of claim 52, wherein the one or more wound is healed at least 10% faster.

54. The method of claim 52, wherein the wound is healed at least 25% faster.

55. The method of claim 42, wherein the subject in need of treatment has or is suspected of having a disease, injury, or disorder with inflammation character or inflammation as a symptom or cause.

56. The method of claim 42, wherein wound healing or inflammation reduction is assessed by histology and/or immunohistochemistry.

57. The method of claim 42, wherein the subject does not experience detectable cytotoxic effects.
